(19) United States
(12) Patent Application Publication
(10) Pub. No.: US 2023/0348518 A1
(43) Pub. Date: Nov. 2, 2023

(54) HELICASE INHIBITORS FOR TREATMENT OF MEDICAL DISORDERS

(71) Applicant: H. LEE MOFFITT CANCER CENTER AND RESEARCH INSTITUTE, INC., Tampa, FL (US)

(72) Inventors: Mark ALEXANDROW, Lutz, FL (US); Nicholas J. LAWRENCE, Lutz, FL (US); Harshani Rithma LAWRENCE, Lutz, FL (US)

(21) Appl. No.: 18/030,186

(22) PCT Filed: Oct. 5, 2021
(86) PCT No.: PCT/US2021/053559

§ 371 Date: Apr. 4, 2023

Related U.S. Application Data
(60) Provisional application No. 63/087,415, filed on Oct. 5, 2020.
(60) Provisional application No. 63/157,268, filed on Mar. 5, 2021.

(51) Int. Cl. C07H 17/04; A61P 35/00

(52) U.S. Cl. CPC C07H 17/04; A61P 35/00

(57) ABSTRACT

This disclosure provides compounds for the treatment of medical disorders such as cancer, and more particularly compounds which find use as inhibitors of CMG helicase.

### CROSS-REFERENCE TO RELATED APPLICATIONS

This application claims the benefit of priority to U.S. Provisional Application No. 63/087,415, filed Oct. 5, 2020, and U.S. Provisional Application No. 63/157,268, filed Mar. 5, 2021, each disclosure of which is incorporated herein by reference in its entirety.

### TECHNICAL FIELD

This disclosure relates to compounds for the treatment of medical disorders such as cancer, and more particularly to compounds which find use as inhibitors of helicases, such as Superfamily 3 (SF3) and Superfamily 6 (SF6) helicases.

### BACKGROUND

Helicases are a class of enzymes that unpack an organism's genes. They are motor proteins that move directionally along a nucleic acid phosphodiester backbone, separating two annealed nucleic acid strands using energy from ATP hydrolysis. There are many helicases representing the great variety of processes in which strand separation must be catalyzed, such as DNA replication, transcription, translation, recombination, DNA repair, and ribosome biogenesis. Helicases are classified in six superfamilies based on their shared sequence motifs; helicases not forming a ring structure are in superfamilies 1 and 2, and ring-forming helicases form part of superfamilies 3 to 6. In particular, superfamily 3 (SF3) consists of AAA+ helicases encoded mainly by small DNA viruses and some large nucleocytoplasmic DNA viruses, with the most known being the papilloma virus E1 helicase. Superfamily 6 (SF6) contain the core AAA+ that are not included in the SF3 classification; some proteins in this group are mini chromosome maintenance MCM, CMG, RuvB, RuvA, and RuvC.

The human replicative helicase functions during DNA replication to melt double-stranded DNA (dsDNA), allowing the polymerases and other replisome components access to single-stranded DNA (ssDNA) for synthesis of daughter strands of DNA. The human replicative helicase is referred to as the CMG helicase, which is derived from the names of its core subunits: Cdc45-MCM complex-GINS complex. MCM subunits are the six Mcm2-7 proteins, and GINS subunits are the four proteins named Go-Ichi-Ni-San (Japanese for 5-1-2-3). The entire CMG holoenzyme is thus composed of 11 subunits, Cdc45, 6 MCM monomers, and 4 GINS subunits, producing a very large ˜750-800 kDa enzyme. Enzymatic activity of the CMG helicase is derived from ATP binding and hydrolysis within the regions between each pair of MCM dimers that make up the MCM hexamer. There are six pairs of MCMs that form six distinct ATP hydrolysis clefts, which work in a non-symmetrical and combinatorial manner to hydrolyze ATP and alter MCM subunit structures to achieve movement along DNA during the melting steps. ATP hydrolysis alters a staircase structure within the central channel of the MCM hexamer through which ssDNA moves in response to changes to this staircase structure during inter-coordinated ATP hydrolysis steps between MCM subunits.

The CMG helicase is an attractive target for cancer drug development due to unique features of its assembly, utilization in cells, and oncogene-induced errors in CMG management that lead to replication dysfunction during tumorigenesis and chemotherapeutic intervention. During the cell cycle mammalian cells are ‘smart’ and predict that they will likely encounter problems during the replicative S-phase, when DNA is being duplicated. For this reason, mammalian cells assemble more CMG helicase precursors, the MCM hexamer, than will be required to complete a normal unperturbed S-phase. An excess of reserve MCM hexamers (˜5× needed) are pre-loaded onto DNA prior to S-phase beginning. Only a subset of these MCM hexamers are chosen, apparently stochastically, to become full CMG helicases upon recruitment of Cdc45 and GINS subunits. Those CMG helicases that form are active during DNA replication. However, if the cell encounters problems such as fork stalling events due to heterochromatin resistance or chemotherapeutic drug exposure that stall forks, then the excess (reserve) MCM hexamers become converted to functional CMG helicases to recover from the fork stalling events. The previous CMG helicases that had been functioning stop unwinding DNA, and these new CMGs become necessary to complete S-phase and the cell cycle. Herein lies the problem in cancers: tumor cells have been found to lack a proper number of unused reserve MCM hexamers and cannot easily create new CMG helicases as needed, for example in recovering from chemotherapy drugs.

There are two currently known mechanisms by which tumor cells mismanage MCM/CMG helicases and thus fail to contain enough reserve helicases. First, overexpression of Cyclin E, which is oncogenic for a number of tumor types, leads to a reduction in MCM hexamer loading onto DNA in tumor cells. This results in a lower yield of total MCM hexamers that could become CMGs for replication or recovery (relative to non-tumor cells that load normal levels of MCMs). Second, Myc overexpression, which is known to occur in 70% or more of human malignancies, produces a related but different effect. Myc is known to be involved in stimulating the assembly and activation of CMG helicases (from MCM hexamers), but too much Myc causes this process to become deregulated and lead to excessive CMG helicase activation. This extra activation of CMG helicases by Myc leads to a loss of unused reserve MCMs, as they have already been turned on by the excess Myc proteins. When a tumor cell with excessive Myc and overactive CMGs is exposed to fork stalling chemotherapy, there are not enough unused reserve MCMs available to mount a healthy response to allow survival of the tumor cells. Again, non-tumor cells do not have elevated Myc expression and do not have elevated CMG activation. Therefore, these two known mechanisms by which oncogenes (Cyclin E or Myc) can mismanage MCM/CMG complexes produces a tumor-selective weakness in CMG levels and recovery from replicative stresses such as fork stalling chemotherapy. These findings also argue that a therapeutic window exists between tumor cells and non-tumor cells in a likely poor response of tumor cells to chemotherapy drugs (for example, as combination approaches using a future CMG inhibitor and chemotherapy). It is predicted from these findings that future CMG inhibitors could provide a unique means of cancer intervention for a variety of cancer types, since the CMG helicase presents an exploitable tumor-specific vulnerability. Note also that other oncogenes besides Myc and Cyclin E could be found to mismanage CMG dynamics in tumor cells, so this concept could extend beyond just these two examples discussed in the above section.

Papillomaviridae is a family of non-enveloped DNA viruses whose members are known as papillomaviruses. Several hundred species of papillomaviruses have been identified infecting all carefully inspected mammals as well as other vertebrates such as birds, snakes, turtles and fish. Infection by most papillomavirus types is either asymptomatic or causes small benign tumors, known a papillomas or warts. Papillomas caused by some papillomavirus types carry a risk of becoming cancerous. Papillomaviruses replicate exclusively in the basal layer of the body surface tissues, with all known papillomavirus types infecting a particular body surface, typically the skin or mucosal epithelium of the genitals, anus, moth, or airways. Papillomaviruses replicate exclusively in keratinocytes, with less-differentiated keratinocyte stem cells thought to be the initial target of productive papillomavirus infections. Subsequent steps in the viral life cycle are strictly dependent on the process of keratinocyte differentiation.

E1, an ATP-dependent DNA helicase, is the only enzyme encoded by papillomaviruses. It is essential for replication and amplification of the viral episome in the nucleus of infected cells. It forms a complex with the viral E2 protein, which is a site-specific DNA-binding transcriptional activator. The E1-E2 complex binds to the replication origin which contains binding sites for both proteins. In addition to E2, it also interacts with DNA polymerase alpha and replication protein A to effect DNA replication. In solution E1 is a monomer, but binds DNA as a dimer. Recruitment of more E1 subunits to the complex leads to melting of the origin and ultimately to the formation of an E1 hexamer with helicase activity.

Human papillomavirus (HPV) infection is caused by HPV, a DNA virus of the Papillomaviridae family. About 90% of HPV infections cause no symptoms and resolve spontaneously within two years. In some cases, an HPV infection persists ands results in either warts or precancerous lesions. These lesions, depending on the site affected, increase the risk of cancer of the cervix, vulva, vagina, penis, anus, mouth, or throat. Over 170 HPV types have been described, with more than 40 able to be spread through sexual contact and infect the anus and genitals. Nearly every individual is infected by HPV at some point in their lives, leading it to being the most common sexually transmitted infection globally.

There is a clear need for the development of treatments for medical disorders, such as cancers, by new mechanisms, such as by inhibition of helicases. The present disclosure addresses this as well as other needs.

### SUMMARY

The present disclosure provides compounds that find use as inhibitors of helicases, such as an SF3 or SF6 helicase, as well as their use in the treatment of medical disorders such as cancer.

Thus, in one aspect, a compound of Formula I is provided:

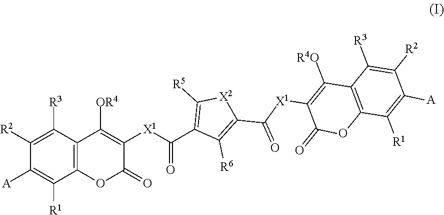

or a pharmaceutically acceptable salt thereof, wherein all variables are as defined further herein.

In another aspect, a compound of Formula II is provided:

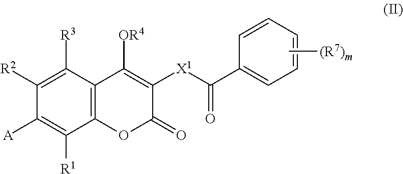

or a pharmaceutically acceptable salt thereof, wherein all variables are as defined further herein.

In yet another aspect, a compound of Formula III is provided:

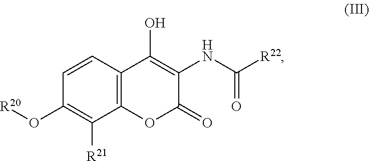

or a pharmaceutically acceptable salt thereof, wherein all variables are as defined herein.

Pharmaceutical compositions are also provided comprising a compound of Formula I, Formula II, or Formula III, or a pharmaceutically acceptable salt thereof, and a pharmaceutically acceptable carrier or excipient.

In another aspect, a method for treating cancer in a subject in need thereof is provided comprising administering to the subject a therapeutically effective amount of a compound of Formula I, Formula II, or Formula III or a pharmaceutically acceptable salt thereof. In some embodiments, the compound of Formula I, Formula II, or Formula III may be administered in combination or alternation with one or more therapeutic agents, for example a chemotherapeutic or cytotoxic agent. In some embodiments, the cancer is associated with or mediated by a helicase, for example an SF3 or an SF6 helicase. In some embodiments, the cancer is associated with overactivation of CMG helicase. In other embodiments, the cancer is associated with an infection by a papillomavirus, for example human papillomavirus (HPV).

In another aspect, a method is provided for treating cancer in as subject in need thereof comprising administering to the subject a therapeutically effective amount of a compound selected from Compound A, Compound B, Compound C, Compound D, and Compound E:

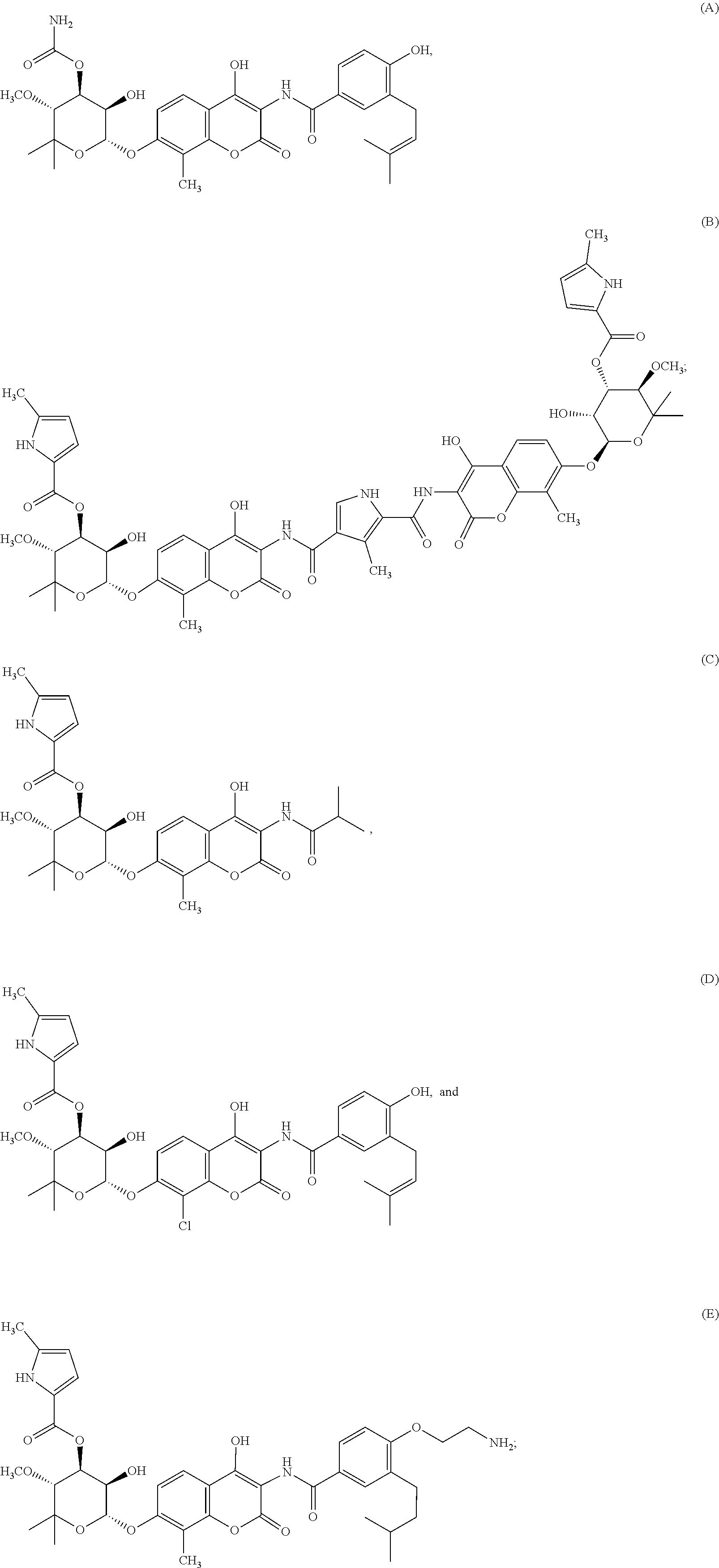

or a pharmaceutically acceptable salt thereof. In some embodiments, the cancer is associated with or mediated by a helicase, for example an SF3 or an SF6 helicase. In some embodiments, the cancer is associated with overactivation of CMG helicase. In other embodiments, the cancer is associated with infection by a papillomavirus, for example human papillomavirus (HPV).

In yet another aspect, a method for treating cancer in a subject in need thereof is provided, the method comprising:
(a) determining whether the cancer is associated with elevated expression of Myc and/or an elevated expression of Cyclin E; and
(b) if the cancer is determined to be associated with elevated expression of Myc and/or an elevated expression of Cyclin E in (a), administering a therapeutically effective amount of a compound of Formula I, Formula II, or Formula III, or a compound selected from Compound A, Compound B, Compound C, Compound D, or Compound E, or a pharmaceutically acceptable salt thereof. In some embodiments, the cancer is associated with overactivation of CMG helicase.

In yet another aspect, a method for treating cancer in a subject in need thereof is provided, wherein the cancer has been previously determined to be associated with elevated expression of Myc and/or an elevated expression of Cyclin E, the method comprising administering a therapeutically effective amount of a compound of Formula I, Formula II, or Formula III, or a compound selected from Compound A, Compound B, Compound C, Compound D, or Compound E, or a pharmaceutically acceptable salt thereof. In some embodiments, the cancer is associated with overactivation of CMG helicase.

In yet another aspect, a method for treating cancer in a subject in need thereof is provided, the method comprising:
(a) determining whether the cancer is associated with one or more signs of replicative stress; and
(b) if the cancer is determined to be associated with one or more signs of replicative stress, administering a therapeutically effective amount of a compound of Formula I, Formula II, or Formula III, or a compound selected from Compound A, Compound B, Compound C, Compound D, or Compound E, or a pharmaceutically acceptable salt thereof.

In another aspect, a method is provided for treating cancer in a subject in need thereof, wherein the cancer has been previously determined to be associated with one or more signs of replicative stress, the method comprising administering a therapeutically effective amount of a compound of Formula I, Formula II, or Formula III, or a compound selected from Compound A, Compound B, Compound C, Compound D, or Compound E, or a pharmaceutically acceptable salt thereof.

In some embodiments, the one or more signs of replicative stress may comprise Myc overexpression, CyclinE overexpression, Rb loss, p53 loss, PolQ overexpression, or combinations thereof.

In yet another aspect, a method for treating cancer in a subject in need thereof is provided, the method comprising:
(a) determining whether the cancer harbors one or more inherited or acquired germ-line mutations; and
(b) if the cancer is determined to harbor one or more inherited or acquired germ-line mutations in (a), administering a therapeutically effective amount of a compound of Formula I, Formula II, or Formula III, or a compound selected from Compound A, Compound B, Compound C, Compound D, or Compound E, or a pharmaceutically acceptable salt thereof.

In another aspect, a method for treating cancer in a subject in need thereof is provided, wherein the cancer has been previously determined to harbor one or more inherited or acquired germ-line mutations, the method comprising administering a therapeutically effective amount of a compound of Formula I, Formula II, or Formula III, or a compound selected from Compound A, Compound B, Compound C, Compound D, or Compound E, or a pharmaceutically acceptable salt thereof.

In some embodiments, the one or more inherited or acquired germ-line mutations may comprise loss of: p53, Rb, BRCA1, BRCA2, ATM, a xeroderma pigmentosum gene (such as XPA, XPB, XPC, XPD, XPE, XPF, or XPG), a mismatch repair gene (such as MSH2, MLH1, MSH6, PMS2), WRN, BLM, a Fanconi anemia gene (such as FANCA, FANCB, FANC, FANCD2, FANCE, FANCF, FANCG, FANCI, FANCJ, FANCL, FANCM, FANCN, FANCO, FANCP, FANCQ, FANCT, FANCU, FANCV, or FANCW), NBS, Chek2, RecqL4, MYH, PALB2, BACH1, RAC51C, or combinations thereof.

In some embodiments, the above methods may further comprise administering one or more additional therapeutic agents. In some embodiments, the one or more additional therapeutic agents may comprise a Chkl inhibitor, an ATR inhibitor, a Cdc7 inhibitor, or a Parp inhibitor.

In another aspect, a method for treating cancer in a subject in need thereof is provided, the method comprising:
(a) administering a therapeutically effective amount of a compound of Formula I, Formula II, or Formula III, or a compound selected from Compound A, Compound B, Compound C, Compound D, or Compound E, or a pharmaceutically acceptable salt thereof; and
(b) administering one or more additional therapeutic agents selected from a Chkl inhibitor, an ATR inhibitor, a Cdc7 inhibitor, and a Parp inhibitor.

In yet another aspect, a method is provided for treating an infection with a papillomavirus in a subject in need thereof comprising administering a therapeutically effective amount of a compound of Formula I, Formula II, or Formula III, or a compound selected from Compound A, Compound B, Compound C, Compound D, or Compound E, or a pharmaceutically acceptable salt thereof. In some embodiments, the papillomavirus is human papillomavirus (HPV).

In yet another aspect, a method is provided for inhibiting a helicase, for example an SF3 and/or an SF6 helicase, in a eukaryotic cell comprising contacting the cell with an effective amount of a compound of Formula I, Formula II, or Formula III, or a compound selected from Compound A, Compound B, Compound C, Compound D, or Compound E, or a pharmaceutically acceptable salt thereof, as described herein. In some embodiments, the eukaryotic cell is a human cell. In some embodiments, the helicase is CMG helicase or HPV E1 helicase.

In yet another aspect, a method is provided for inhibiting replication of a papillomavirus, for example HPV, in a eukaryotic cell comprising contacting the cell with an effective amount of a compound of Formula I, Formula II, or Formula III, or a compound selected from Compound A, Compound B, Compound C, Compound D, or Compound E, or a pharmaceutically acceptable salt thereof, as described herein. In some embodiments, the eukaryotic cell is a human cell.

The details of one or more embodiments of the disclosure are set forth in the accompanying drawings and the description below. Other features, objects, and advantages of the disclosure will be apparent from the description and drawings, and from the claims.

### DESCRIPTION OF DRAWINGS

FIGS. 1A and 1B show coumermycin A1 (Compound B) inhibiting RPA loading onto DNA/chromatin, while novobiocin (Compound A) does not inhibit RPA at all.

FIG. 2 shows that Simian virus 40 (SV40) large-T-antigen (Tag) DNA helicase and the human CMG helicase are both capable of being inhibited directed in vitro (by a classic biochemical fork unwinding assay) by coumermycin-A1 (Compound B) but not novobiocin (Compound A).

FIG. 3 shows the results of a fluorescent-polarization assay showing coumermycin A1 (Compound B) and clorobiocin (Compound D) inhibiting CMG helicase, while novobiocin (Compound A) does not.

FIG. 4 shows the structure and ATP clefts of CMG helicase.

FIGS. 5A and 5B show that tumor cells lack reserve MCMs/CMGs due to Myc overexpression.

FIGS. 6A, 6B, 6C, 6D, 6E, and 6F show that acute Myc expression sensitizes tumor cells to replicative stresses. BT-549 breast tumor cells with normal Myc levels were infected with retroviruses expressing extra MycER proteins. Mut-MycER lacks MB-II. Activation of MycER (+40HT) is known to cause excessive origin/CMG usage and this mut-MycER does not. At doses of drugs that cause few adverse effects without MycER activation (no-OHT), extra Myc function sensitizes to both drugs (but not with mut-MycER). (A) Protein expression verification. (B) Experimental design. (C&D) Aphidicolin tested by cfu assays. (E&F) Gemcitabine tested. Averages from triplicated plating+/−1 s.d are shown.

FIGS. 7A and 7B show that tumor cells lack reserve MCMs/CMGs due to cyclin E overexpression.

FIGS. 8A, 8B, 8C, and 8D show that PDAC cells (Panc-1) are selectively chemosensitized to gemcitabine by reduction of CMG reserves, versus immortalized non-tumor HaCaT cells.siRNA partial suppression of CMGs (thus reducing reserves) via loss of Mcm7 subunit was performed for 120 hours (5 days). Gemcitabine (or no drug) was added to clutures from 72-120 hours (days 4-5). Colonies were allowed to form (or not) for two weeks, then counted. (A&B) Western blots showing partial loss of Mcm7 in both cell types during the 72-120 hour window of concurrent Gem exposure. (C&D) Results of clonogenic assays after siRNA+Gem exposures. Duplicate plates for each condition in clonogenic assays were scored on two fields each, and results were averaged, +/−1 SD. Asterisks represent p-values, where one is <0.05 and two is <0.01. Note for ‘no drug’ samples that siRNA against Mcm7 does not appreciably perturb cell growth, indicating that we only suppressed reserve CMGs/MCMs. HaCaT were chosen as a comparison due to their similar cell cycling index to Panc-1 (must be growing well, versus primary cells which grow slower). Data suggest a therapeutic window and/or feasibility exists for anti-CMG intervention in these (and other) cancers.

FIGS. 9A, 9B, 9C, and 9D show success purifying active hCMG from insect cell expression methods. (A) The approach used is very similar to that published by Kang and Hurwitz and yields similar amounts of hCMG. (B) Immunoblotting of glycerol fractions obtained from a ˜1.5-2.0 liter prep of hCMG. Most hCMG is present in HMW fractions near antibodies against human proteins. (C) A silver stain of purified hCMG from infected Sf9 cells immediately after the first bead step; compared to non-infected control Sf9 lysates from same beads. (D) A radioactive helicase fork unwinding assay was performed on fraction #7 and showed very active and titratable hCMG enzyme activity from the prep. It was compared to purified SV40 Tag helicase purified separated. The other fractions were tested, and hCMG activity mirrors the Western blot results, where hCMG presence correlated with activity.

FIGS. 10A and 10B show tests that purified hCMG can function in a fluorescent-polarization (FP) assay measuring ADP production quantitatively. (A) Diagram showing how the FP assay works in measuring ADP production by the hCMG. Assay reagents are designed/sold by BellBrook Labs (WI, USA) for assessing ADP production in drug screening regiments. Conditions for the enzyme were developed based on company instructions and customized for the enzyme due to complexity of the hCMG having multiple subunits. A Perkin Elmer EnVision-II dual-FP plate reader is used for analysis. Fluorescent light is in the far-red range. (B) Preliminary evidence for the hCMG being successfully used in this FP assay, within the readable range of the window defined by free-tracer vs tracer+Ab at optimized levels. Data are plotted as change in milli-polarizations (delta-mP), normalized to the window defined as 100%=max free-tracer output (max ADP production capable of being sensed). Note that 2 microliters of hCMG prep gives ˜52% delta-MP value, with a Z′-factor of ˜0.6.

FIG. 11 shows a titration of coumermycin-A1 (Compound B) inhibiting SV40 Tag helicase in a classic fork unwinding assay.

FIG. 12 shows that the E1 helicase from HPV16 can be directly inhibited by coumermycin-A1 (Compound B) and novobiocin (Compound A).

FIGS. 13A, 13B, 13C, 13D, and 13E show that osteosarcoma (OS) tumor cells are sensitized to chemotherapy drugs if CMG helicase is co-suppressed.

FIGS. 14A, 14B, 14C, 14D, 14E, and 14F shows that CMG inhibition (with coumermycin-A1) inhibits OS tumor cells with more specificity than non-tumor cells (Hacat). Novobiocin does not inhibit any cells. The OS tumor cells have gene deficiencies that render the CMG helicase as a vulnerability, which Hacat do not have these gene problems (i.e., are normal cells). Coumermycin-A1 inhibits MCM assembly and activity and Hacat cells and OS tumor cells (143b). Coumermycin-A1 also causes DNA damage specifically in tumor cells, which leads to Parp cleavage.

FIGS. 15A and 15B show that CMG helicase inhibition (with coumermycin-A1) suppresses OS tumor growth in mice (12 day analysis).

FIGS. 16A, 16B, an 16C show that CMG helicase inhibition (with coumermycin-A1) suppresses OS tumor growth in mice (18 day analysis).

FIG. 17 shows multiple tumor types sensitive to low doses of CMG helicase inhibition (with coumermycin-A1). Non-tumor Hacat cells are far less sensitive due to not harboring genetic abnormalities that make CMG a vulnerability.

FIG. 18 shows H82 cells (small cell lung cancer) are also sensitive to CMG helicase inhibition. These cells have Myc overexpression and Rb loss. MCM assembly is inhibited by CMG helicase inhibition.

FIG. 19 shows that Hacat non-tumor skin cells are far less sensitive to CMG helicase inhibition (with coumermycin-A1).

FIG. 20 shows that HPV31 E1 helicase (full-length) is inhibited by coumermycin-A1.

FIG. 21 shows that HPV16 E1 helicase (full-length) is inhibited by coumermycin-A1.

FIG. 22 shows that HPV18 E1 helicase (helicase domain only) is inhibited by coumermycin-A1.

FIG. 23 shows that HPV16 E1 helicase (helicase domain only) is inhibited by coumermycin-A1.

FIG. 24 shows that clorobiocin inhibits human CMG (hCMG) in a dose-dependent manner.

FIG. 25 shows that clorobiocin inhibits human CMG (hCMG) in direct comparison to novobiocin (500 uM).

Like reference symbols in the various drawings indicate like elements.

### DETAILED DESCRIPTION

The following description of the disclosure is provided as an enabling teaching of the disclosure in its best, currently known embodiments. Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As can be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It can be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of”.

As used in the specification and the appended claims, the singular forms “a,” “an” and “the” include plural referents unless the context clearly dictates otherwise. Thus, for example, reference to “a compound”, “a pharmaceutical composition”, or “a cancer”, includes, but is not limited to, two or more such compounds, pharmaceutical compositions, or cancers, and the like.

It should be noted that ratios, concentrations, amounts, and other numerical data can be expressed herein in a range format. It can be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it can be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of a monomer refers to an amount that is sufficient to achieve the desired improvement in the property modulated by the formulation component, e.g. desired antioxidant release rate or viscoelasticity. The specific level in terms of wt % in a composition required as an effective amount will depend upon a variety of factors including the amount and type of monomer, amount and type of polymer, e.g., acrylamide, amount of antioxidant, and desired release kinetics.

As used herein, the term “therapeutically effective amount” refers to an amount that is sufficient to achieve the desired therapeutic result or to have an effect on undesired symptoms but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed drug delivery composition can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the terms “optional” or “optionally” means that the subsequently described event or circumstance can or cannot occur, and that the description includes instances where said event or circumstance occurs and instances where it does not.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as an ophthalmological disorder. The effect can be therapeutic in terms of a partial or complete cure of a disease, condition, symptom or adverse effect attributed to the disease, disorder, or condition. The term “treatment” as used herein can include any treatment of ophthalmological disorder in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “therapeutic” can refer to treating, healing, and/or ameliorating a disease, disorder, condition, or side effect, or to decreasing in the rate of advancement of a disease, disorder, condition, or side effect.

### Chemical Definitions

Compounds are described using standard nomenclature. Unless defined otherwise, all technical and scientific terms used herein have the same meaning as is commonly understood by one of skill in the art to which this invention belongs.

The compounds described herein include enantiomers, mixtures of enantiomers, diastereomers, tautomers, racemates and other isomers, such as rotamers, as if each is specifically described, unless otherwise indicated or otherwise excluded by context.

A dash (“-”) that is not between two letters or symbols is used to indicate a point of attachment for a substituent. For example, —(C═O)NH₂ is attached through the carbon of the keto (C═O) group.

The term “substituted”, as used herein, means that any one or more hydrogens on the designated atom or group is replaced with a moiety selected from the indicated group, provided that the designated atom's normal valence is not exceeded and the resulting compound is stable. For example, when the substituent is oxo (i.e., ═O) then two hydrogens on the atom are replaced. For example, a pyridyl group substituted by oxo is a pyridine. Combinations of substituents and/or variables are permissible only if such combinations result in stable compounds or useful synthetic intermediates. A stable active compound refers to a compound that can be isolated and can be formulated into a dosage form with a shelf life of at least one month. A stable manufacturing intermediate or precursor to an active compound is stable if it does not degrade within the period needed for reaction or other use. A stable moiety or substituent group is one that does not degrade, react or fall apart within the period necessary for use. Non-limiting examples of unstable moieties are those that combine heteroatoms in an unstable arrangement, as typically known and identifiable to those of skill in the art.

Any suitable group may be present on a “substituted” or “optionally substituted” position that forms a stable molecule and meets the desired purpose of the invention and includes, but is not limited to: alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, silyl, sulfo-oxo, sulfonyl, sulfone, sulfoxide, sulfonylamino, or thiol.

“Alkyl” is a straight chain or branched saturated aliphatic hydrocarbon group. In certain embodiments, the alkyl is C₁-C₂, C₁-C₃, or C₁-C₆ (i.e., the alkyl chain can be 1, 2, 3, 4, 5, or 6 carbons in length). The specified ranges as used herein indicate an alkyl group with length of each member of the range described as an independent species. For example, C₁-C₆alkyl as used herein indicates an alkyl group having from 1, 2, 3, 4, 5, or 6 carbon atoms and is intended to mean that each of these is described as an independent species and C₁-C₄alkyl as used herein indicates an alkyl group having from 1, 2, 3, or 4 carbon atoms and is intended to mean that each of these is described as an independent species. When C₀-Cₙalkyl is used herein in conjunction with another group, for example (C₃-C₇cycloalkyl)C₀-C₄alkyl, or —C₀-C₄(C₃-C₇cycloalkyl), the indicated group, in this case cycloalkyl, is either directly bound by a single covalent bond (C₀alkyl), or attached by an alkyl chain, in this case 1, 2, 3, or 4 carbon atoms. Alkyls can also be attached via other groups such as heteroatoms, as in —O—C₀-C₄alkyl(C₃-C₇cycloalkyl). Examples of alkyl include, but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentane, 3-methylpentane, 2,2-dimethylbutane, and 2,3-dimethylbutane. In one embodiments, the alkyl group is optionally substituted as described herein.

“Cycloalkyl” is a saturated mono- or multi-cyclic hydrocarbon ring system. When composed of two or more rings, the rings may be joined together in a fused or bridged fashion. Non-limiting examples of typical cycloalkyl groups include cyclopropyl, cyclobutyl, cyclopentyl, cyclohexyl, and cycloheptyl. In one embodiment, the cycloalkyl group is optionally substituted as described herein.

“Alkenyl” is a straight or branched chain aliphatic hydrocarbon group having one or more carbon-carbon double bonds, each of which is independently either cis or trans, that may occur at a stable point along the chain. Non-limiting examples include C₂-C₄alkenyl and C₂-C₆alkenyl (i.e., having 2, 3, 4, 5, or 6 carbons). The specified ranges as used herein indicate an alkenyl group having each member of the range described as an independent species, as described above for the alkyl moiety. Examples of alkenyl include, but are not limited to, ethenyl and propenyl. In one embodiment, the alkenyl group is optionally substituted as described herein.

“Alkynyl” is a straight or branched chain aliphatic hydrocarbon group having one or more carbon-carbon triple bonds that may occur at any stable point along the chain, for example, C₂-C₄alkynyl or C₂-C₆alkynyl (i.e., having 2, 3, 4, 5, or 6 carbons). The specified ranges as used herein indicate an alkynyl group having each member of the range described as an independent species, as described above for the alkyl moiety. Examples of alkynyl include, but are not limited to, ethynyl, propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, and 5-hexynyl. In one embodiment, the alkynyl group is optionally substituted as described herein.

“Alkoxy” is an alkyl group as defined above covalently bound through an oxygen bridge (—O—). Examples of alkoxy include, but are not limited to, methoxy, ethoy, n-propoxy, isopropoxy, n-butoxy, 2-butoxy, tert-butoxy, n-pentoxy, 2-pentoxy, 3-pentoxy, isopentoxy, neopentoxy, n-hexoxy, 2-hexoxy, 3-hexoxy, and 3-methylpentoxy. Similarly, an “alkylthio” or “thioalkyl” group is an alkyl group as defined above with the indicated number of carbon atoms covalently bound through a sulfur bridge (—S—). In one embodiment, the alkoxy group is optionally substituted as described herein.

“Alkanoyl” is an alkyl group as defined above covalently bound through a carbonyl (C═O) bridge. The carbonyl carbon is included in the number of carbons, for example C₂alkanoyl is a CH₃(C═O)— group. In one embodiment, the alkanoyl group is optionally substituted as described herein.

“Haloalkoxy” indicates a haloalkyl group as defined herein attached through an oxygen bridge (oxygen of an alcohol radical).

“Halo” or “halogen” indicates, independently, any of fluoro, chloro, bromo or iodo.

“Aryl” indicates an aromatic group containing only carbon in the aromatic ring or rings. In one embodiment, the aryl group contains 1 to 3 separate or fused rings and is 6 to 14 or 18 ring atoms, without heteroatoms as ring members. When indicated, such aryl groups may be further substituted with carbon or non-carbon atoms or groups. Such substitution may include fusion to a 4- to 7- or 5- to 7-membered saturated or partially unsaturated cyclic group that optionally contains 1, 2, or 3 heteroatoms independently selected from N, O, B, P, Si and S, to form, for example, a 3,4-methylenedioxyphenyl group. Aryl groups include, for example, phenyl and naphthyl, including 1-naphthyl and 2-naphthyl. In one embodiment, aryl groups are pendant. An example of a pendant ring is a phenyl group substituted with a phenyl group. In one embodiment, the aryl group is optionally substituted as described herein.

The term “heterocycle” refers to saturated and partially saturated heteroatom-containing ring radicals, where the heteroatoms may be selected from N, O, and S. The term heterocycle includes monocyclic 3-12 members rings, as well as bicyclic 5-16 membered ring systems (which can include fused, bridged, or spiro bicyclic ring systems). It does not include rings containing —O—O—, —O—S—, and —S—S— portions. Examples of saturated heterocycle groups including saturated 4- to 7-membered monocyclic groups containing 1 to 4 nitrogen atoms [e.g., pyrrolidinyl, imidazolidinyl, piperidinyl, pyrrolinyl, azetidinyl, piperazinyl, and pyrazolidinyl]; saturated 4- to 6-membered monocyclic groups containing 1 to 2 oxygen atoms and 1 to 3 nitrogen atoms [e.g., morpholinyl]; and saturated 3- to 6-membered heteromonocyclic groups containing 1 to 2 sulfur atoms and 1 to 3 nitrogen atoms [e.g., thiazolidinyl]. Examples of partially saturated heterocycle radicals include, but are not limited, dihydrothienyl, dihydropyranyl, dihydrofuryl, and dihydrothiazolyl. Examples of partially saturated and saturated heterocycle groups include, but are not limited to, pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3,-dihydro-1H-benzo[d]isothazol-6-yl, dihydropyranyl, dihydrofuryl, and dihydrothiazolyl. Bicyclic heterocycle includes groups wherein the heterocyclic radical is fused with an aryl radical wherein the point of attachment is the heterocycle ring. Bicyclic heterocycle also includes heterocyclic radicals that are fused with a carbocyclic radical. Representative examples include, but are not limited to, partially unsaturated condensed heterocyclic groups containing 1 to 5 nitrogen atoms, for example indoline and isoindoline, partially unsaturated condensed heterocyclic groups containing 1 to 2 oxygen atoms and 1 to 3 nitrogen atoms, partially unsaturated condensed heterocyclic groups containing 1 to 2 sulfur atoms and 1 to 3 nitrogen atoms, and saturated condensed heterocyclic groups containing 1 to 2 oxygen or sulfur atoms.

“Heteroaryl” refers to a stable monocyclic, bicyclic, or multicyclic aromatic ring which contains from 1 to 3, or in some embodiments 1, 2, or 3 heteroatoms selected from N, O, S, B, and P (and typically selected from N, O, and S) with remaining ring atoms being carbon, or a stable bicyclic or tricyclic system containing at least one 5, 6, or 7 membered aromatic ring which contains from 1 to 3, or in some embodiments from 1 to 2, heteroatoms selected from N, O, S, B, or P, with remaining ring atoms being carbon. In one embodiments, the only heteroatom is nitrogen. In one embodiment, the only heteroatom is oxygen. In one embodiment, the only heteroatom is sulfur. Monocyclic heteroaryl groups typically have from 5 to 6 ring atoms. In some embodiments, bicyclic heteroaryl groups are 8- to 10-membered heteroaryl groups, that is groups containing 8 or 10 ring atoms in which one 5-, 6-, or 7-membered aromatic ring is fused to a second aromatic or non-aromatic ring, wherein the point of attachment is the aromatic ring. When the total number of S and O atoms in the heteroaryl group excess 1, these heteroatoms are not adjacent to one another. In one embodiment, the total number of S and O atoms in the heteroaryl group is not more than 2. In another embodiment, the total number of S and O atoms in the heteroaryl group is not more than 1. Examples of heteroaryl groups include, but are not limited to, pyridinyl, imidazolyl, imidazopyridinyl, pyrimidinyl, pyrazolyl, triazolyl, pyrazinyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, triazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl.

A “pharmaceutically acceptable salt” is a derivative of the disclosed compound in which the parent compound is modified by making inorganic and organic, pharmaceutically acceptable, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include salts which are acceptable for human consumption and the quaternary ammonium salts of the parent compound formed, for example, from inorganic or organic salts. Example of such salts include, but are not limited to, those derived from inorganic acids such as hydrochloric, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicyclic, mesylic, esylic, besylic, sulfanilic, 2-acetoxybenzoic, fumaric, toluenesulfonic, methanesulfone, ethane disulfonic, oxalic, isethionic, HOOC—(CH₂)₁₄—COOH, and the like, or using a different acid that produced the same counterion. Lists of additional suitable salts may be found, e.g., in Remington's Pharmaceutical Sciences, 17^{th} ed., Mack Publishing Company, Easton, PA., p. 1418 (1985).

### Compounds of Formula I, Formula II, and Formula III

The present disclosure provides compounds which may be useful as inhibitors of helicases, such as SF3 and/or SF6 helicases, and more particularly, for example, CMG helicase and HPV E1 helicase. The present compounds have utility in the treatment of medical disorders mediated by an SF3 and/or an SF6 helicase, for example cancer.

In one aspect, a compound of Formula I is provided:

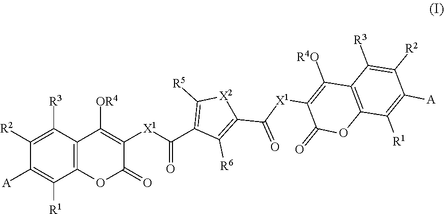

or a pharmaceutically acceptable salt thereof,
wherein:
A is

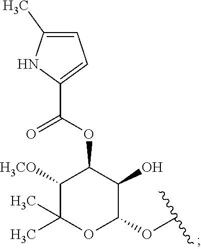 [SMILES: *O[C@@H]1OC(C)(C)[C@H](OC)[C@@H](OC(=O)c2ccc(C)[nH]2)[C@H]1O]

R¹, R², and R³ are independently selected at each occurrence from hydrogen, halo, nitro, cyano, azido, C₁-C₆alkyl, C₁-C₆haloalkyl, C₂-C₆alkenyl, C₂-C₆alkynyl, C₃-C₇cycloalkyl, (4- to 10-membered monocyclic or bicyclic heterocycle)-(C₀-C₆alkyl), (5- to 10-membered monocyclic or bicyclic aryl)-(C₀-C₆alkyl), (5- to 10-membered monocyclic or bicyclic heteroaryl)-(C₀-C₆alkyl), —OR^{a}, —SR^{a}, —NR^{a}R^{b}, —C(O)R^{c}, —S(O)R^{c}, and —S(O)₂R^{c}, each of which may be optionally substituted with one or more Y groups as allowed by valency;
R⁴ is selected at each occurrence from hydrogen, C₁-C₆alkyl, C₃-C₇cycloalkyl, (4- to 10-membered monocyclic or bicyclic heterocycle)-(C₀-C₆alkyl), (5- to 10-membered monocyclic or bicyclic aryl)-(C₀-C₆alkyl), or (5- to 10-membered monocyclic or bicyclic heteroaryl)-(C₀-C₆alkyl), each of which may be optionally substituted with one or more Y groups as allowed by valency;
with the proviso that if R⁴ is hydrogen, then R³ is not methyl;
R⁵ and R⁶ are each independently hydrogen, halo, nitro, cyano, azido, C₁-C₆alkyl, C₁-C₆haloalkyl, C₂-C₆alkenyl, C₂-C₆alkynyl, C₃-C₇cycloalkyl, (4- to 10-membered monocyclic or bicyclic heterocycle)-(C₀-C₆alkyl), (5- to 10-membered monocyclic or bicyclic aryl)-(C₀-C₆alkyl), (5- to 10-membered monocyclic or bicyclic heteroaryl)-(C₀-C₆alkyl), —OR^{a}, —SR^{a}, —NR^{a}Rb, —C(O)R^{c}, —S(O)R^{c}, and —S(O)₂R^{c}, each of which may be optionally substituted with one or more Y groups as allowed by valency;
X¹ is independently selected at each occurrence from —O—, —NH—, or —N(R^{d})—;
X² is —O—, —NH—, —N(R^{d})—, or —S—;
R^{a} and R^{b} are independently selected at each occurrence from hydrogen, C₁-C₆alkyl, C₁-C₆haloalkyl, C₂-C₆alkenyl, C₂-C₆alkynyl, C₃-C₇cycloalkyl, (4- to 10-membered monocyclic or bicyclic heterocycle)-(C₀-C₆alkyl), (5- to 10-membered monocyclic or bicyclic aryl)-(C₀-C₆alkyl), and (5- to 10-membered monocyclic or bicyclic heteroaryl)-(C₀-C₆alkyl), each of which may be optionally substituted with one or more Y groups as allowed by valency;
R^{c} is independently selected at each occurrence from hydrogen, halo, C₁-C₆alkyl, C₁-C₆haloalkyl, C₂-C₆alkenyl, C₂-C₆alkynyl, C₃-C₇cycloalkyl, (4- to 10-membered monocyclic or bicyclic heterocycle)-(C₀-C₆alkyl), (5- to 10-membered monocyclic or bicyclic aryl)-(C₁-C₆alkyl), (5- to 10-membered monocyclic or bicyclic heteroaryl)-(C₀-C₆alkyl), —OR^{a}, —SR^{a}, and —NR^{a}R^{b}, each of which may be optionally substituted with one or more Y groups as allowed by valency;
R^{d} is independently selected at each occurrence from hydrogen, C₁-C₆alkyl, C₁-C₆haloalkyl, C₃-C₇cycloalkyl, 4- to 10-membered monocyclic or bicyclic heterocycle, 5- to 10-membered monocyclic or bicyclic aryl, or 5- to 10-membered monocyclic or bicyclic heteroaryl, each of which may be optionally substituted with one or more Y groups as allowed by valency; and
Y is independently selected at each occurrence from halo, hydroxy, amino, cyano, —CHO, —COOH, —CONH₂, C₁-C₆alkyl, C₂-C₆alkanoyl, (mono or di-C₁-C₆alkylamino)C₀-C₂alkyl, C₁-C₆haloalkyl, hydroxyC₁-C₆alkyl, ester, carbamate, urea, sulfonamide, —C₁-C₆alkyl(4- to 10-membered monocyclic or bicyclic heterocycle), —C₁-C₆alkyl(5- to 10-membered monocyclic or bicyclic heteroaryl), —C₁-C₆alkyl(C₃-C₇cycloalkyl), 0-C₁-C₆alkyl(C₃-C₇cycloalkyl), B(OH)₂, phosphate, phosphonate, and C₁-C₆haloalkoxy.

In some embodiments, the compound of Formula I is selected from a compound of Formula I-a:

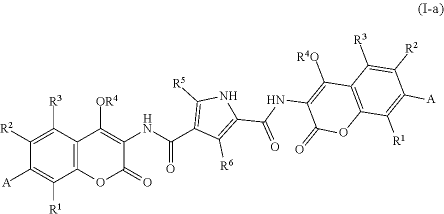

or a pharmaceutically acceptable salt thereof, wherein all variables are as defined herein.

In some embodiments, the compound of Formula I is selected from a compound of

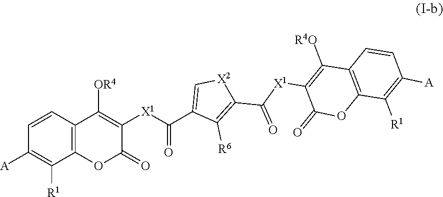

or a pharmaceutically acceptable salt thereof, wherein all variables are as defined herein.

In another aspect, a compound of Formula II is provided:

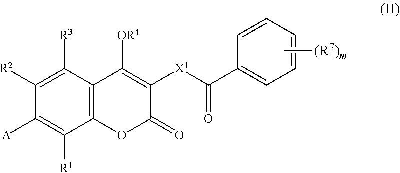

or a pharmaceutically acceptable salt thereof,
wherein:
m is 0, 1, 2, 3, 4, or 5;
R⁷ is independently selected at each occurrence from halo, nitro, cyano, azido, C₁-C₆alkyl, C₁-C₆haloalkyl, C₂-C₆alkenyl, C₂-C₆alkynyl, C₃-C₇cycloalkyl, (4- to 10-membered monocyclic or bicyclic heterocycle)-(C₀-C₆alkyl), (5- to 10-membered monocyclic or bicyclic aryl)-(C₀-C₆alkyl), (5- to 10-membered monocyclic or bicyclic heteroaryl)-(C₀-C₆alkyl), —OR^{a}, —SR^{a}, —NR^{a}R^{b}, —C(O)R^{c}, —S(O)R^{c}, and —S(O)₂R^{c}, each of which may be optionally substituted with one or more Y groups as allowed by valency;
with the proviso that if R⁴ is hydrogen, R¹ cannot be chloro;
and all other variables are as defined herein.

In some embodiments, the compound of Formula II is selected from a compound of Formula II-a:

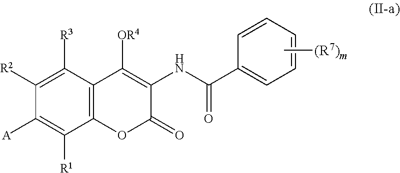

or a pharmaceutically acceptable salt thereof, wherein all variables are as defined herein.

In some embodiments, the compound of Formula II is selected from a compound of Formula II-b:

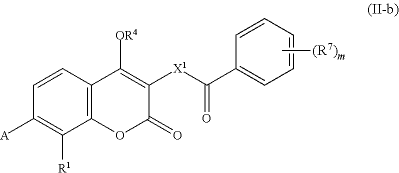

or a pharmaceutically acceptable salt thereof, wherein all variables are as defined herein.

In some embodiments of Formula I or Formula II, R¹ is hydrogen. In some embodiments of Formula I or Formula II, R¹ is halo, for example fluoro, chloro, or bromo. In some embodiments of Formula I or Formula II, R¹ is cyano. In some embodiments of Formula I or Formula II, R¹ is nitro. In some embodiments of Formula I or Formula II, R¹ is C₁-C₆alkyl, for example methyl or ethyl. In some embodiments of Formula I or Formula I, R¹ is C₁-C₆haloalkyl, for example trifluoromethyl. In some embodiments of Formula I or Formula II, R¹ is selected from C₃-C₇cycloalkyl, for example cyclopropyl or cyclobutyl. In some embodiments of Formula I or Formula II, R¹ is selected from —OR^{a}, wherein R^{a} is selected from hydrogen, methyl, ethyl, or trifluoromethyl. In some embodiments of Formula I or Formula II, R¹ is selected from —NR^{a}R^{b}, wherein R^{a} and R^{b} are each independently hydrogen or methyl.

In some embodiments of Formula I or Formula II, R² is hydrogen. In some embodiments of Formula I or Formula II, R² is halo, for example fluoro, chloro, or bromo. In some embodiments of Formula I or Formula II, R² is cyano. In some embodiments of Formula I or Formula II, R² is nitro. In some embodiments of Formula I or Formula II, R² is C₁-C₆alkyl, for example methyl or ethyl. In some embodiments of Formula I or Formula I, R² is C₁-C₆haloalkyl, for example trifluoromethyl. In some embodiments of Formula I or Formula II, R² is selected from C₃-C₇cycloalkyl, for example cyclopropyl or cyclobutyl. In some embodiments of Formula I or Formula II, R² is selected from —OR^{a}, wherein R^{a} is selected from hydrogen, methyl, ethyl, or trifluoromethyl. In some embodiments of Formula I or Formula II, R² is selected from —NR^{a}R^{b}, wherein R^{a} and R^{b} are each independently hydrogen or methyl.

In some embodiments of Formula I or Formula II, R³ is hydrogen. In some embodiments of Formula I or Formula II, R³ is halo, for example fluoro, chloro, or bromo. In some embodiments of Formula I or Formula II, R³ is cyano. In some embodiments of Formula I or Formula II, R³ is nitro. In some embodiments of Formula I or Formula II, R³ is C₁-C₆alkyl, for example methyl or ethyl. In some embodiments of Formula I or Formula I, R³ is C₁-C₆haloalkyl, for example trifluoromethyl. In some embodiments of Formula I or Formula II, R³ is selected from C₃-C₇cycloalkyl, for example cyclopropyl or cyclobutyl. In some embodiments of Formula I or Formula II, R³ is selected from —OR^{a}, wherein R^{a} is selected from hydrogen, methyl, ethyl, or trifluoromethyl. In some embodiments of Formula I or Formula II, R³ is selected from —NR^{a}R^{b}, wherein R^{a} and R^{b} are each independently hydrogen or methyl.

In some embodiments of Formula I or Formula II, R⁴ is hydrogen. In some embodiments of Formula I or Formula II, R⁴ is C₁-C₆ alkyl, for example methyl, ethyl, or isopropyl. In some embodiments of Formula I or Formula II, R⁴ is phenyl.

In some embodiments of Formula I or Formula II, X¹ is —NH—. In some embodiments of Formula I or Formula II, X¹ is —N(CH₃)—.

In some embodiments of Formula I, R⁵ is hydrogen. In some embodiments of Formula I, R⁵ is halo, for example fluoro, chloro, or bromo. In some embodiments of Formula I, R⁵ is cyano. In some embodiments of Formula I, R⁵ is nitro. In some embodiments of Formula I, R⁵ is C₁-C₆alkyl, for example methyl or ethyl. In some embodiments of Formula I, R⁵ is C₁-C₆haloalkyl, for example trifluoromethyl. In some embodiments of Formula I, R⁵ is selected from C₃-C₇cycloalkyl, for example cyclopropyl or cyclobutyl. In some embodiments of Formula I I, R⁵ is selected from —OR^{a}, wherein R^{a} is selected from hydrogen, methyl, ethyl, or trifluoromethyl. In some embodiments of Formula I, R⁵ is selected from —NR^{a}R^{b}, wherein R^{a} and R^{b} are each independently hydrogen or methyl.

In some embodiments of Formula I, R⁶ is hydrogen. In some embodiments of Formula I, R⁶ is halo, for example fluoro, chloro, or bromo. In some embodiments of Formula I, R⁶ is cyano. In some embodiments of Formula I, R⁶ is nitro. In some embodiments of Formula I, R⁶ is C₁-C₆alkyl, for example methyl or ethyl. In some embodiments of Formula I, R⁶ is C₁-C₆haloalkyl, for example trifluoromethyl. In some embodiments of Formula I, R⁶ is selected from C₃-C₇cycloalkyl, for example cyclopropyl or cyclobutyl. In some embodiments of Formula I, R⁶ is selected from —OR^{a}, wherein R^{a} is selected from hydrogen, methyl, ethyl, or trifluoromethyl. In some embodiments of Formula I, R⁶ is selected from —NR^{a}R^{b}, wherein R^{a} and R^{b} are each independently hydrogen or methyl.

In some embodiments of Formula I, X² is —O—. In some embodiments of Formula I, X² is —NH—. In some embodiments of Formula I, X² is —N(CH₃)—. In some embodiments of Formula I, X² is —S—.

In some embodiments of Formula II, m is 0. In some embodiments of Formula II, m is 1. In some embodiments of Formula II, m is 2. In some embodiments of Formula II, m is 3. In some embodiments of Formula II, m is 4. In some embodiments of Formula II, m is 5.

In some embodiments of Formula II, R⁷ is halo, for example fluoro, chloro, or bromo. In some embodiments of Formula II, R⁷ is cyano. In some embodiments of Formula II, R⁷ is nitro. In some embodiments of Formula II, R⁷ is C₁-C₆alkyl, for example methyl or ethyl. In some embodiments of Formula II, R⁷ is C₁-C₆haloalkyl, for example trifluoromethyl. In some embodiments of Formula II, R⁷ is selected from C₃-C₇cycloalkyl, for example cyclopropyl or cyclobutyl. In some embodiments of Formula I, R⁷ is selected from —OR^{a}, wherein R^{a} is selected from hydrogen, methyl, ethyl, or trifluoromethyl. In some embodiments of Formula II, R⁷ is selected from —NR^{a}R^{b}, wherein R^{a} and R^{b} are each independently hydrogen or methyl.

In an alternative aspect, a compound of Formula III is provided:

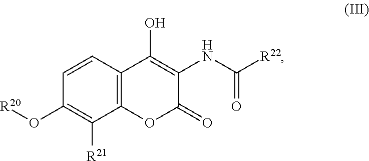

or a pharmaceutically acceptable salt thereof;
wherein:
R²⁰ is selected from R^{20a}, R^{20b}, and R^{20c};
R^{20a} is selected from 5- to 6-membered cycloalkyl, 5- to 6-membered heterocyclyl, aryl, or 5- to 6-membered heteroaryl, wherein R^{20a} is substituted with R³⁰ and is optionally substituted with one or more Y¹ groups;
R^{20b} is 5- to 6-membered heterocyclyl having N(R³¹) as a ring atom and optionally substituted with one or more Y¹ groups;
R^{20c} is

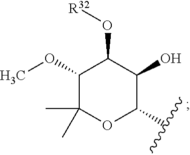

R³⁰ is selected from —NH(C═O)OR^{a1}, —O(C═O)N(R^{a2})(R^{a3}), —NH(C═O)N(R^{a2})(R^{a3}) —NH(C═O)R^{a4}, and OR³²;
R³¹ is selected from —C(═O)OR^{a1}, —C(═O)N(R^{a2})(R^{a3}), —C(═S)N(R^{a2})(R^{a3}), —S(═O)N(R^{a2})(R^{a3}), —S(O)₂N(R^{a2})(R^{a3}), and —C(═O)R^{a4};
R³² is selected from —C(═O)(5- to 10-membered monocyclic or bicyclic heteroaryl) and —C(═O)(5- to 10-membered monocyclic or bicyclic heterocyclyl), each of which R³² may be optionally substituted with one or more Z¹ groups and each of which R³² may optionally contain N(Z²) as a ring heteroatom;
R²¹ is selected from C₁-C₆ alkyl and halo;
R²² is selected from C₁-C₆ alkyl, (C₃-C₆ cycloalkyl)-(C₀-C₃ alkyl)-, C₁-C₆ haloalkyl,
R^{22a}, and R^{22b};
R^{22a} is

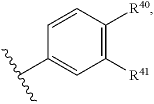

wherein R^{22a} is optionally substituted with one or more Y² groups;
R^{22b} is 5- to 6-membered heterocyclyl having N(R⁴²) as a ring atom and optionally substituted with one or more Y² groups;
R⁴⁰ is selected from hydrogen, halo, nitro, cyano, azido, —OR^{b1}, and —N(R^{b1})(R^{b2});
R⁴¹ is selected from hydrogen, C₁-C₆ alkyl, C₁-C₆ haloalkyl, C₂-C₆ alkenyl, C₂-C₆ alkynyl, (C₃-C₆ cycloalkyl)-(C₀-C₃ alkyl)-, C₁-C₆ alkoxy, and (C₃-C₆ cycloalkyl)-(C₀-C₃ alkyl)-O—;
R⁴² is selected from hydrogen, C₁-C₆ alkyl, (C₃-C₆ cycoalkyl)-(C₀-C₃ alkyl), and hydroxy(C₁-C₆ alkyl)-;
R^{a1} is selected from hydrogen, C₁-C₆ alkyl, C₂-C₆ alkenyl, C₂-C₆ alkynyl, (C₃-C₆ cycloalkyl)-(C₀-C₃ alkyl)-, (3- to 10-membered monocyclic or bicyclic heterocyclyl)-(C₀-C₃ alkyl)-, (C₆-C₁₀ monocyclic or bicyclic aryl)-(C₀-C₃ alkyl), and (5- to 10-membered monocyclic or bicyclic heteroaryl)-(C₀-C₃ alkyl)-, each of which may be substituted with one or more Q groups;
R^{a2} and R^{a3} are independently selected from hydrogen, C₁-C₆ alkyl, (C₃-C₆ cycloalkyl)-(C₀-C₃ alkyl)-, and (C₆-C₁₀ aryl)-(C₀-C₃ alkyl)-, each of which may be substituted with one or more Q groups;
R^{a4} is selected from hydrogen, C₁-C₆ alkyl, C₂-C₆ alkenyl, C₂-C₆ alkynyl, (C₃-C₆ cycloalkyl)-(C₀-C₃ alkyl)-, (3- to 10-membered monocyclic or bicyclic heterocyclyl)-(C₀-C₃ alkyl)-, (C₆-C₁₀ monocyclic or bicyclic aryl)-(C₀-C₃ alkyl), and (5- to 10-membered monocyclic or bicyclic heteroaryl)-(C₀-C₃ alkyl)-, each of which may be substituted with one or more Q groups;
R^{b1} and R^{b2} are independently selected at each occurrence from hydrogen, C₁-C₆ alkyl, C₂-C₆ alkenyl, C₂-C₆ alkynyl, and (C₆-C₁₀ aryl)-(C₀-C₃ alkyl)-;
Q is independently selected at each occurrence from hydrogen, halo, nitro, cyano, azido, C₁-C₆ alkyl, C₂-C₆ alkenyl, C₂-C₆ alkynyl, C₁-C₆ alkoxy, (C₃-C₆ cycloalkyl)-(C₀-C₃ alkyl)-, and (C₆-C₁₀ aryl)-(C₀-C₃ alkyl);
Y¹ is independently selected at each occurrence from hydrogen, halo, nitro, cyano, azido, —OR^{b1}, —N(R^{b1})(R^{b2}), C₁-C₆ alkyl, C₁-C₆ haloalkyl, C₂-C₆ alkenyl, C₂-C₆ alkynyl, —(C₁-C₃ alkyl)-OR^{b1}, and —(C₁-C₃ alkyl)-N(R^{b1})(R^{b2});
or two Y¹ groups on the same carbon are brought together with the carbon to which they are attached form an oxo or vinylidene group;
Y² is independently selected at each occurrence from hydrogen, halo, nitro, cyano, azido, —OR^{b1}, —N(R^{b1})(R^{b2}), C₁-C₆ alkyl, C₁-C₆ haloalkyl, C₂-C₆ alkenyl, C₂-C₆ alkynyl, —(C₁-C₃ alkyl)-OR^{b1}, and —(C₁-C₃ alkyl)-N(R^{b1})(R^{b2});
or two Y² groups on the same carbon are brought together with the carbon to which they are attached form an oxo or vinylidene group;
Z¹ is independently selected at each occurrence from halo, nitro, cyano, azido, —OR^{b1}, —N(R^{b1})(R^{b2}), C₁-C₆ alkyl, C₂-C₆ haloalkyl, C₂-C₆ alkenyl, C₂-C₆ alkynyl, (C₃-C₆ cycloalkyl)-(C₀-C₃ alkyl)-, (3- to 6-membered monocyclic or bicyclic heterocyclyl)-(C₀-C₃ alkyl)-, (C₆-C₁₀ aryl)-(C₀-C₃ alkyl)-, and (5- to 10-membered monocyclic or bicyclic heteroaryl)-(C₀-C₃ alkyl)-, each of which may be optionally substituted with one or more Q groups; and
Z² is independently selected at each occurrence from C₁-C₆ alkyl, C₂-C₆ haloalkyl, C₂-C₆ alkenyl, C₂-C₆ alkynyl, (C₃-C₆ cycloalkyl)-(C₀-C₃ alkyl)-, (3- to 6-membered monocyclic or bicyclic heterocyclyl)-(C₀-C₃ alkyl)-, (C₆-C₁₀ aryl)-(C₀-C₃ alkyl)-, and (5- to 10-membered monocyclic or bicyclic heteroaryl)-(C₀-C₃ alkyl)-, each of which may be optionally substituted with one or more Q groups.

In some embodiments of Formula III, R²⁰ is R^{20a}. In some embodiments, R^{20a} is 5- to 6-membered cycloalkyl substituted with R³⁰ and optionally substituted with 1, 2, 3, or 4 Y¹ groups. In some embodiments of Formula III, R^{20a} is 5- to 6-membered heterocyclyl substituted with R³⁰ and optionally substituted with 1, 2, 3, or 4 Y¹ groups. In some embodiments of Formula III, R^{20a} is aryl substituted with R³⁰ and optionally substituted with 1, 2, 3, or 4 Y¹ groups. In some embodiments of Formula III, R^{20a} is 5- to 6-membered heteroaryl substituted with R³⁰ and optionally substituted with 1, 2, 3, or 4 Y¹ groups.

In some embodiments of Formula III, R^{20a} is selected from:

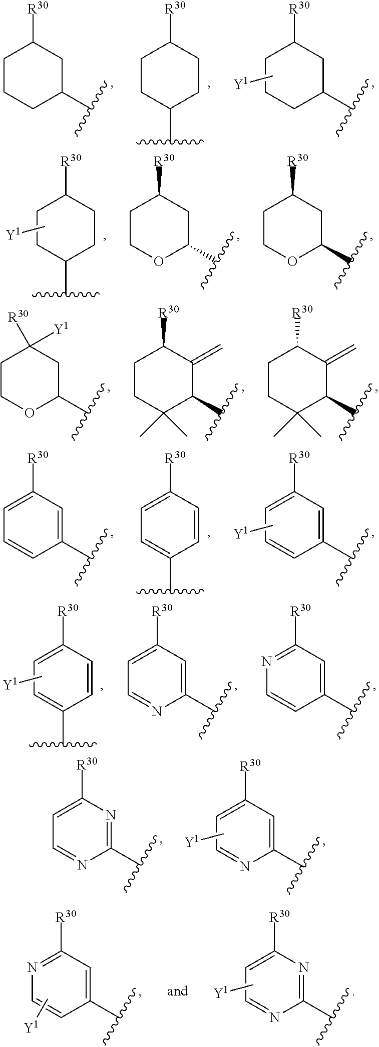

In some embodiments of Formula III, R^{20a} is selected from:

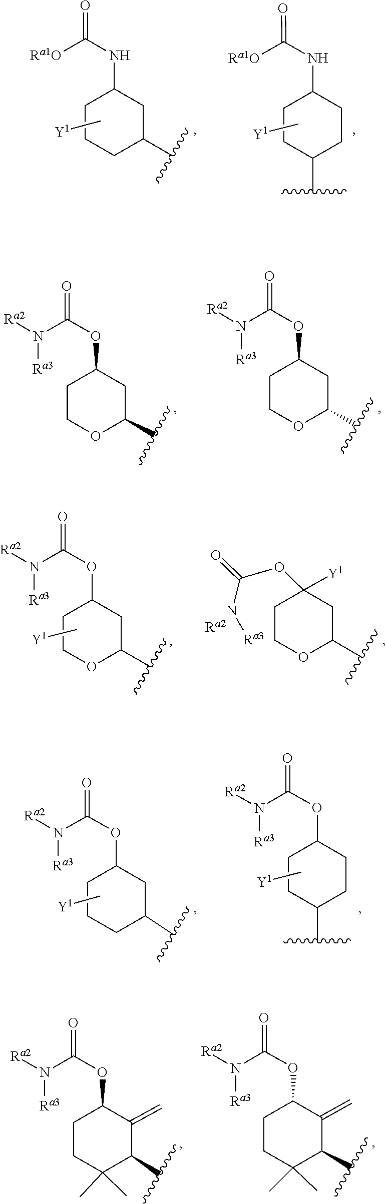

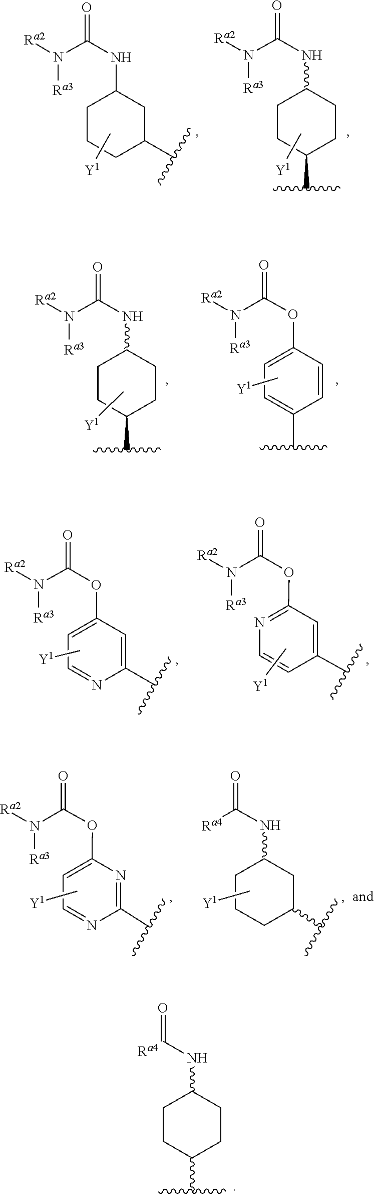

In some embodiments of Formula III, R²⁰ is R^{20b}.

In some embodiments of Formula III, R^{20b} is selected from:

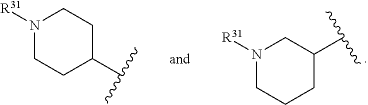

In some embodiments of Formula III, R^{20b} is selected from:

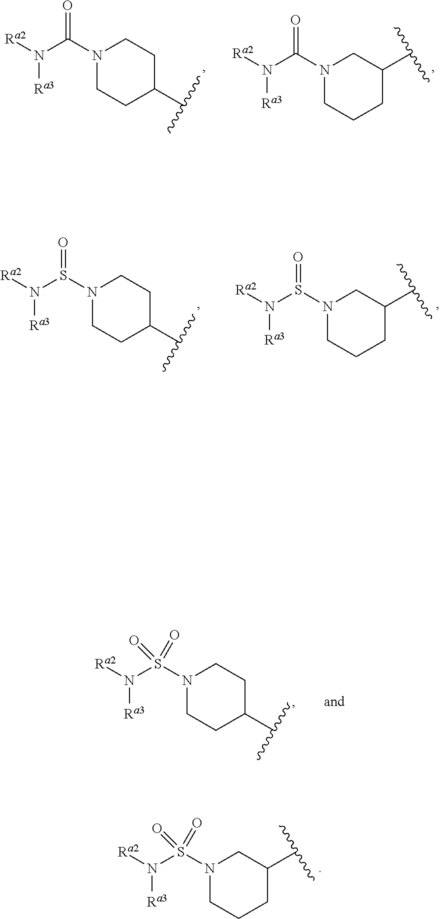

In some embodiments of Formula III, R²⁰ is R^{20c}.

In some embodiments of Formula III, R³² is selected from:

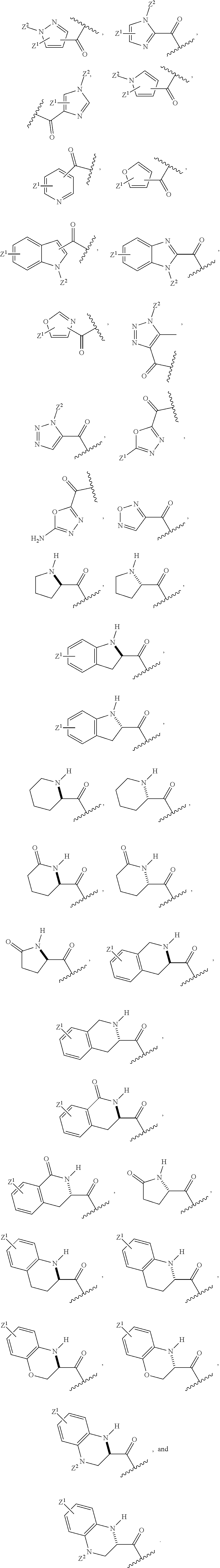

In some embodiments of Formula III, Y¹ is hydrogen. In some embodiments of Formula III, Y¹ is —OH. In some embodiments of Formula III, Y¹ is F. In some embodiments of Formula III, Y¹ is Cl. In some embodiments of Formula III, Y¹ is Br. In some embodiments of Formula III, Y¹ is I. In some embodiments of Formula III, Y¹ is OMe. In some embodiments of Formula III, Y¹ is —NH₂. In some embodiments of Formula III, Y¹ is —O-(benzyl). In some embodiments of Formula III, Y¹ is —OEt. In some embodiments of Formula III, Y¹ is —O-(propargyl). In some embodiments of Formula III, Y¹ is propargyl. In some embodiments of Formula III, Y¹ is methyl. In some embodiments of Formula III, Y¹ is ethyl. In some embodiments of Formula III, Y¹ is propyl. In some embodiments of Formula III, Y¹ is —CF₃. In some embodiments of Formula III, Y¹ is —N(independently alkyl)₂. In some embodiments of Formula III, Y¹ is —NH(alkyl). In some embodiments of Formula III, Y¹ is CN. In some embodiments of Formula III, Y¹ is halogen.

In some embodiments of Formula III, Y¹ is —CH₂Br. In some embodiments of Formula III, Y¹ is —CH₂OH. In some embodiments of Formula III, Y¹ is —CH₂OMe. In some embodiments of Formula III, Y¹ is —CH₂NH₂. In some embodiments of Formula III, Y¹ is —CH₂NHMe. In some embodiments of Formula III, Y¹ is —CH₂N(Me)₂.

In some embodiments of Formula III, R^{a1} is hydrogen. In some embodiments of Formula III, R^{a1} is alkyl. In some embodiments of Formula III, R^{a1} is methyl. In some embodiments of Formula III, R^{a1} is ethyl. In some embodiments of Formula III, R^{a1} is propyl. In some embodiments of Formula III, R^{a1} is isopropyl. In some embodiments of Formula III, R^{a1} is isobutyl. In some embodiments of Formula III, R^{a1} is cyclopropyl. In some embodiments of Formula III, R^{a1} is propargyl. In some embodiments of Formula III, R^{a1} is tert-butyl. In some embodiments of Formula III, R^{a1} is cyclopentyl. In some embodiments of Formula III, R^{a1} is benzyl. In some embodiments of Formula III, R^{a1} is heterocyclyl. In some embodiments of Formula III, R^{a1} is aryl. In some embodiments of Formula III, R^{a1} is heteroaryl.

In some embodiments of Formula III, R^{a2} is hydrogen. In some embodiments of Formula III, R^{a2} is methyl. In some embodiments of Formula III, R^{a2} is ethyl. In some embodiments of Formula III, R^{a2} is propyl. In some embodiments of Formula III, R^{a2} is cyclopropyl. In some embodiments of Formula III, R^{a2} is —CH₂(cyclopropyl). In some embodiments of Formula III, R^{a2} is phenyl. In some embodiments of Formula III, R^{a2} is benzyl. In some embodiments of Formula III, R^{a2} is phenyl substituted with one or more substituents selected from fluoro, chloro, bromo, iodo, nitro, cyano, amino, methyl or ethyl.

In some embodiments of Formula III, R^{a3} is hydrogen. In some embodiments of Formula III, R^{a3} is methyl. In some embodiments of Formula III, R^{a3} is ethyl. In some embodiments of Formula III, R^{a3} is propyl. In some embodiments of Formula III, R^{a3} is cyclopropyl. In some embodiments of Formula III, R^{a3} is —CH₂(cyclopropyl). In some embodiments of Formula III, R^{a3} is phenyl. In some embodiments of Formula III, R^{a3} is benzyl. In some embodiments of Formula III, R^{a3} is phenyl substituted with one or more substituents selected from fluoro, chloro, bromo, iodo, nitro, cyano, amino, methyl or ethyl.

In some embodiments of Formula III, R^{a4} is hydrogen. In some embodiments of Formula III, R^{a4} is alkyl. In some embodiments of Formula III, R^{a4} is methyl. In some embodiments of Formula III, R^{a4} is ethyl. In some embodiments of Formula III, R^{a4} is propyl. In some embodiments of Formula III, R^{a4} is isopropyl. In some embodiments of Formula III, R^{a4} is isobutyl. In some embodiments of Formula III, R^{a4} is cyclopropyl. In some embodiments of Formula III, R^{a4} is propargyl. In some embodiments of Formula III, R^{a4} is tert-butyl. In some embodiments of Formula III, R^{a4} is cyclopentyl. In some embodiments of Formula III, R^{a4} is benzyl. In some embodiments of Formula III, R^{a4} is heterocyclyl. In some embodiments of Formula III, R^{a4} is aryl. In some embodiments of Formula III, R^{a4} is heteroaryl.

In some embodiments of Formula III, Z¹ is —OH. In some embodiments of Formula III, Z¹ is alkyl. In some embodiments of Formula III, Z¹ is methyl. In some embodiments of Formula III, Z¹ is propyl. In some embodiments of Formula III, Z¹ is butyl. In some embodiments of Formula III, Z¹ is isobutyl. In some embodiments of Formula III, Z¹ is cyclopropyl. In some embodiments of Formula III, Z¹ is phenyl. In some embodiments of Formula III, Z¹ is propargyl. In some embodiments of Formula III, Z¹ is aryl. In some embodiments of Formula III, Z¹ is heteroaryl. In some embodiments of Formula III, Z¹ is cycloalkyl. In some embodiments of Formula III, Z¹ is —O(alkyl). In some embodiments of Formula III, Z¹ is halo. In some embodiments of Formula III, Z¹ is fluoro. In some embodiments of Formula III, Z¹ is chloro. In some embodiments of Formula III, Z¹ is 4-methoxybenzyl. In some embodiments of Formula III, Z¹ is benzyl substituted with one or more halo substituents.

In some embodiments of Formula III, Z² is hydrogen. In some embodiments of Formula III, Z² is alkyl. In some embodiments of Formula III, Z² is methyl. In some embodiments of Formula III, Z² is propyl. In some embodiments of Formula III, Z² is butyl. In some embodiments of Formula III, Z² is isobutyl. In some embodiments of Formula III, Z² is cyclopropyl. In some embodiments of Formula III, Z² is phenyl. In some embodiments of Formula III, Z² is propargyl. In some embodiments of Formula III, Z² is —OMe. In some embodiments of Formula III, Z² is phenyl substituted with one more alkyl and/or halo substituents. In some embodiments of Formula III, Z² is benzyl.

In some embodiments of Formula III, R²¹ is methyl. In some embodiments of Formula III, R²¹ is chloro.

In some embodiments of Formula III, R²² is selected from:

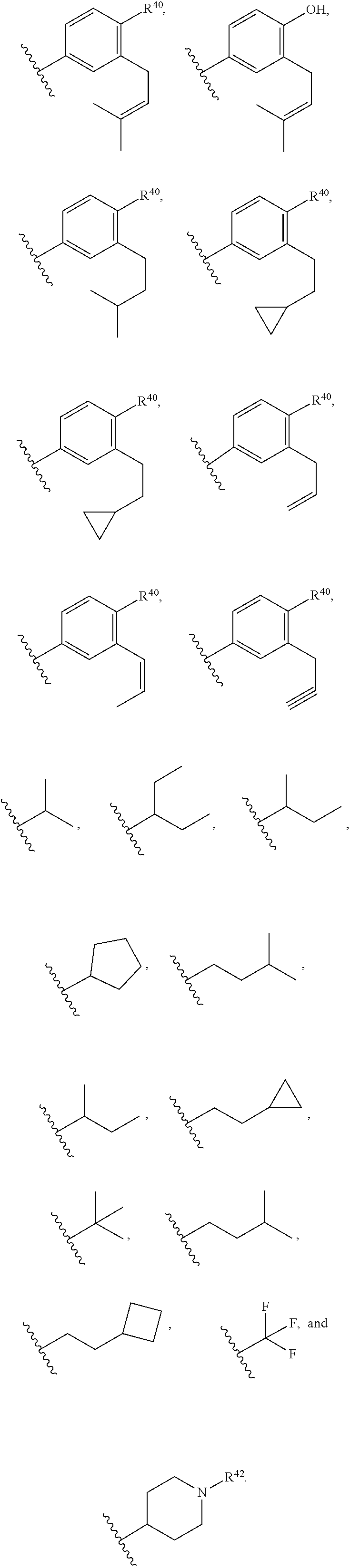

In some embodiments of Formula III, R⁴⁰ is hydrogen. In some embodiments of Formula III, R⁴⁰ is —OH. In some embodiments of Formula III, R⁴⁰ is —O(alkyl). In some embodiments of Formula III, R⁴⁰ is Cl. In some embodiments of Formula III, R⁴⁰ is F. In some embodiments of Formula III, R⁴⁰ is CN. In some embodiments of Formula III, R⁴⁰ is —NH(alkyl). In some embodiments of Formula III, R⁴⁰ is N(independently alkyl)₂.

In some embodiments of Formula III, R⁴¹ is alkyl. In some embodiments of Formula III, R⁴¹ is cycloalkyl. In some embodiments of Formula III, R⁴¹ is —O(alkyl). In some embodiments of Formula III, R⁴¹ is —O(cycloalkyl).

In some embodiments of Formula III, R⁴² is alkyl. In some embodiments of Formula III, R⁴² is cyclopropyl. In some embodiments of Formula III, R⁴² is —CH₂CH₂OH.

In some embodiments, the compound of Formula III is a compound of Formula III-a OH H

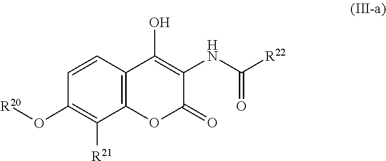

or a pharmaceutically acceptable salt thereof;
wherein:
R²⁰ is selected from:

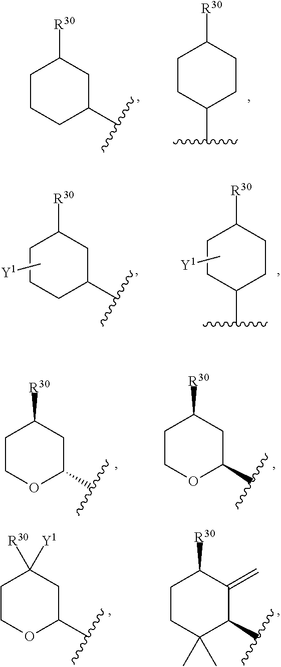

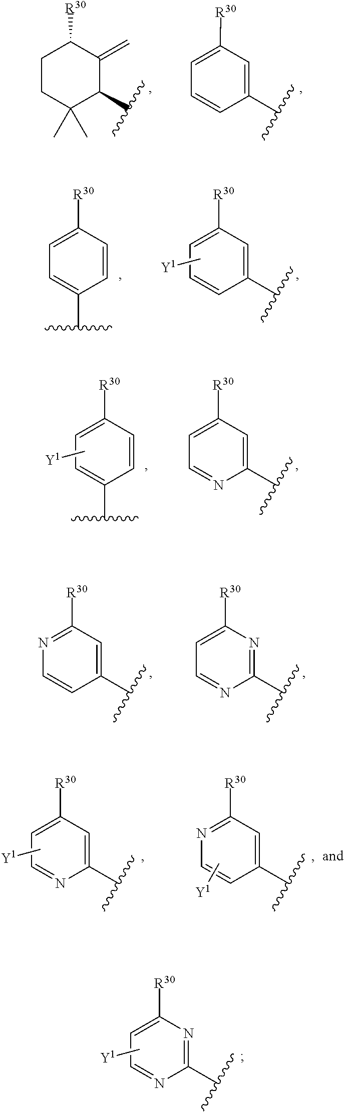

R²¹ is methyl or chloro;
R²² is selected from:

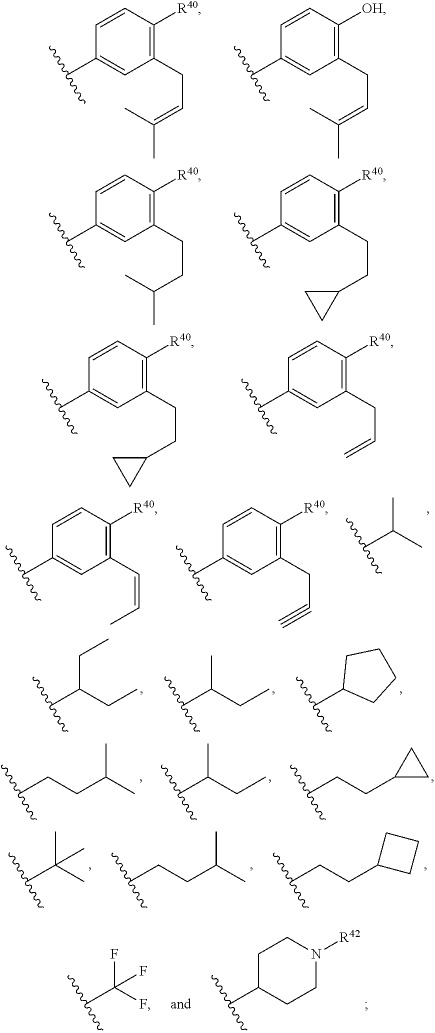

and all other variables are as defined herein.

In some embodiments, the compound of Formula III is a compound of Formula III-b

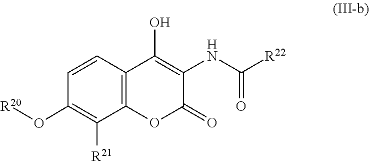

or a pharmaceutically acceptable salt thereof;
wherein:
R²⁰ is selected from:

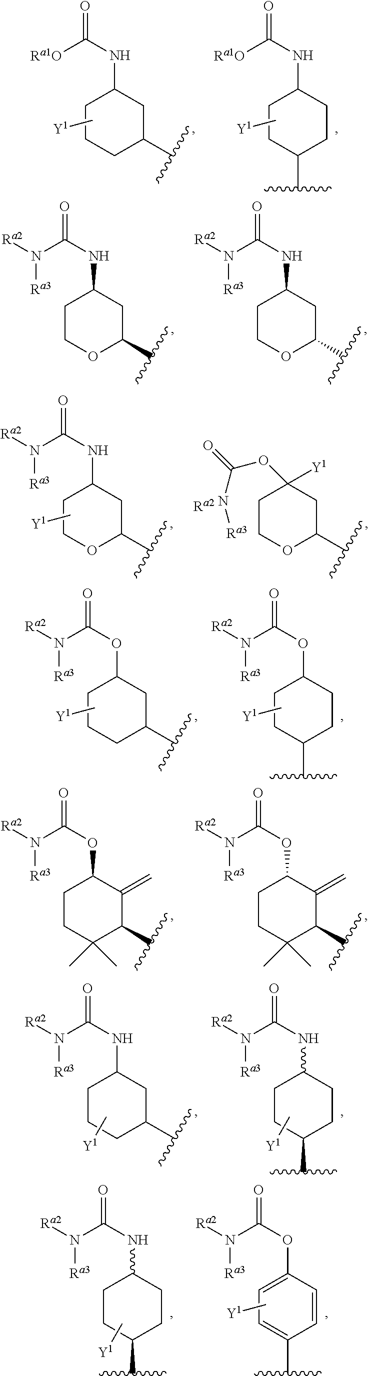

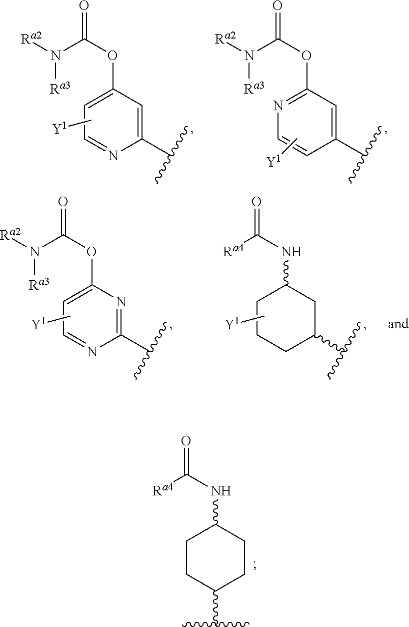

R²¹ is methyl or chloro;
R²² is selected from:

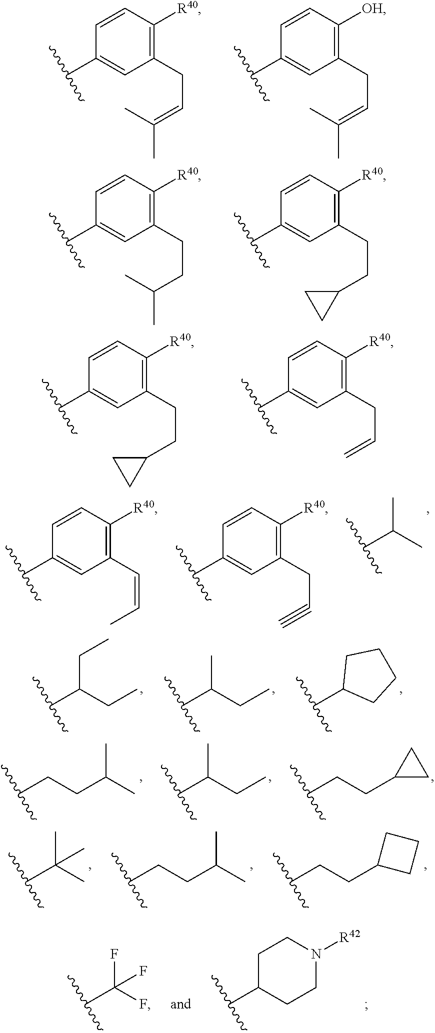

and all other variables are as defined herein.

In some embodiments, the compound of Formula III is a compound of Formula III-c

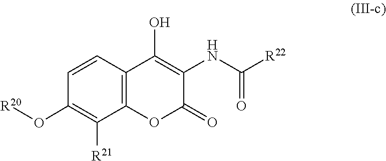

or a pharmaceutically acceptable salt thereof;
wherein:
R²⁰ is selected from

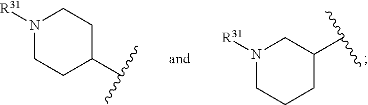

R²¹ is methyl or chloro;
R²² is selected from

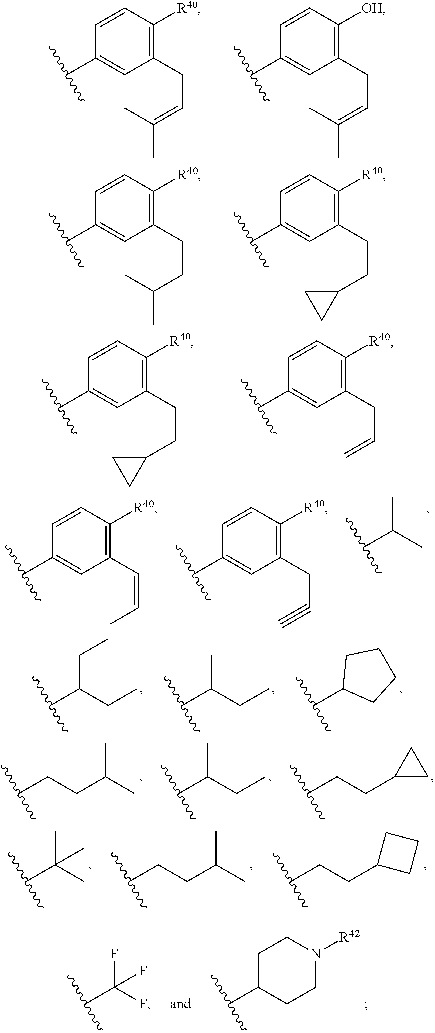

and all other variables are as defined herein.

In some embodiments, the compound of Formula III is a compound of Formula III-d

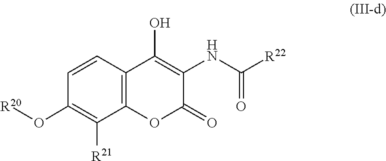

or a pharmaceutically acceptable salt thereof;
wherein:
R²⁰ is selected from

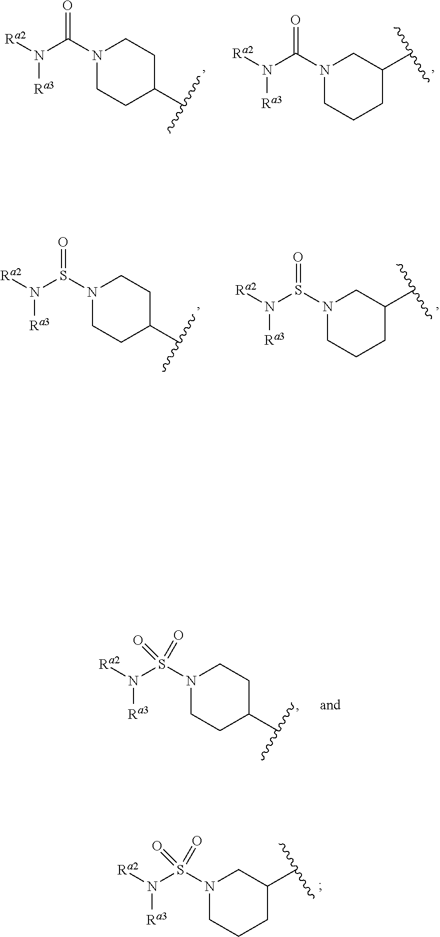

R²¹ is methyl or chloro;
R²² is selected from

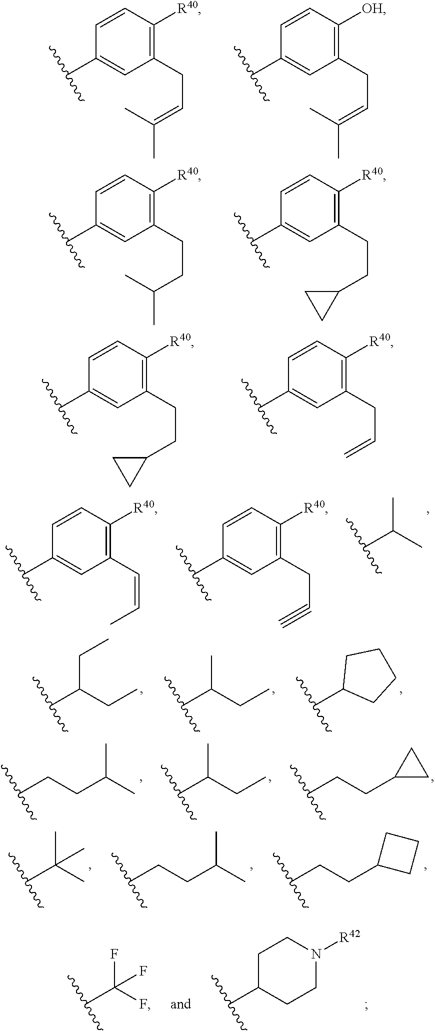

and all other variables are as defined herein.

In some embodiments, the compound of Formula III is a compound of Formula III-e

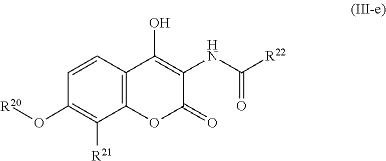

or a pharmaceutically acceptable salt thereof;
wherein:
R²⁰ is

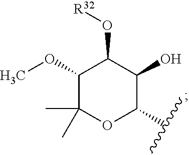

R³² is selected from

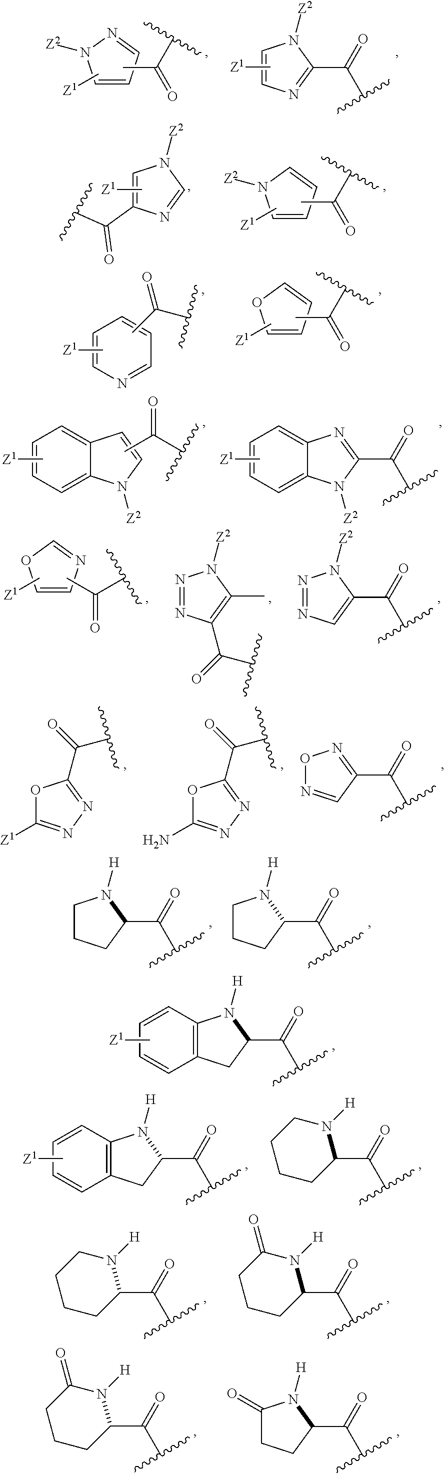

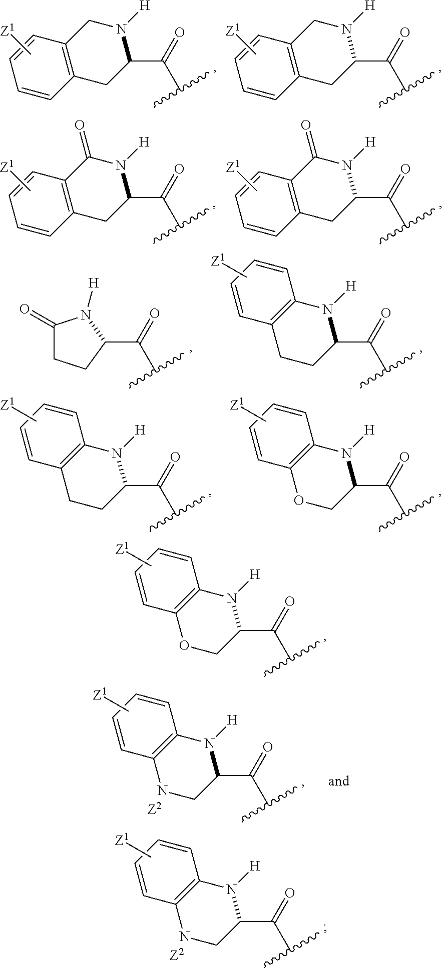

R²¹ is methyl or chloro;
R²² is selected from

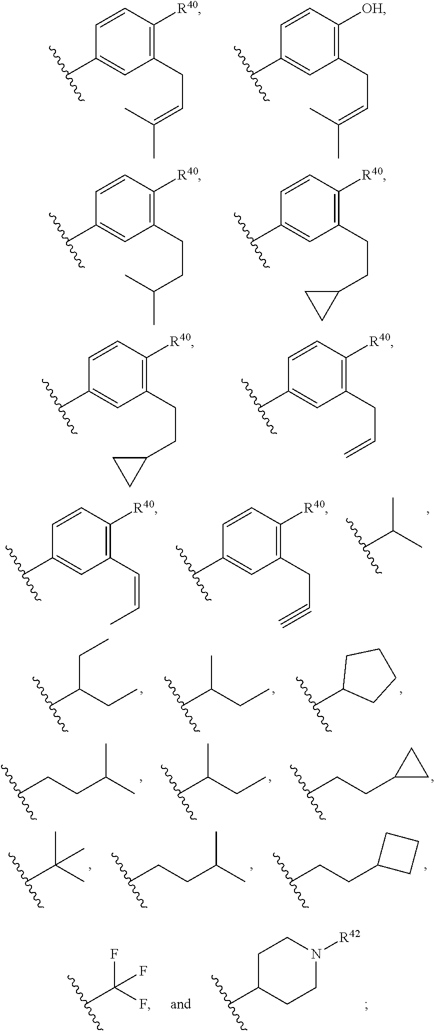

and all other variables are as defined herein.

Representative compounds of Formula III may be synthesized according to the following scheme:

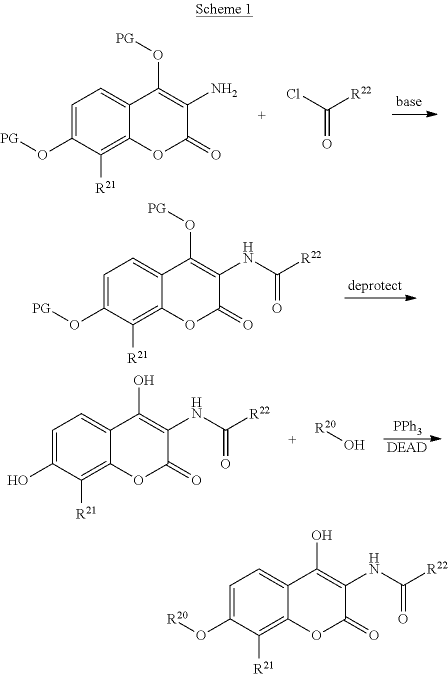

Representative compounds of Formula III-e may be synthesized according to the following scheme:

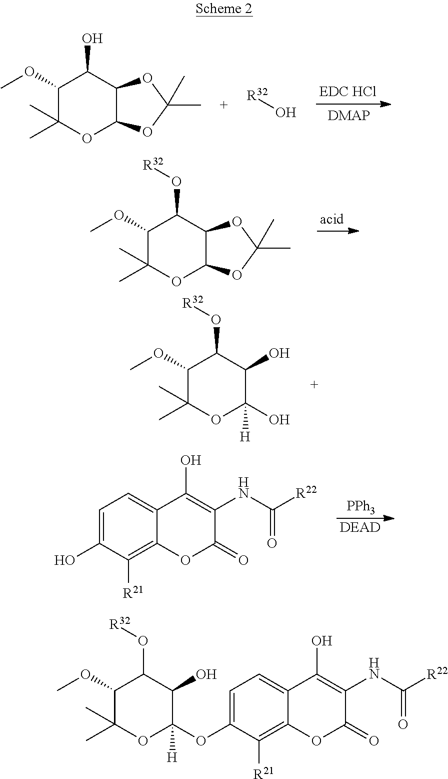

The present disclosure also includes compounds of Formula I, Formula II, or Formula III with at least one desired isotopic substitution of an atom, at an amount above the natural abundance of the isotope, i.e., enriched.

Examples of isotopes that can be incorporated into compounds of the present disclosure include isotopes of hydrogen, carbon, nitrogen, oxygen, phosphorous, fluorine, and chlorine, such as ²H, ³H, ¹¹C, ¹³C, ¹⁵N, ¹⁷ O, ¹⁸O, ¹⁸F, ³¹P, ³²P, ³⁵S, ³⁶Cl, and ¹²⁵I respectively. In one embodiment, isotopically labeled compounds can be used in metabolic studies (with ¹⁴C), reaction kinetic studies (with, for example ²H or ³H), detection or imaging techniques, such as positron emission tomography (PET) or single-photon emission computed tomography (SPECT) including drug and substrate tissue distribution assays, or in radioactive treatment of patients. In particular, an ¹⁸F labeled compound may be particularly desirable for PET or SPECT studies. Isotopically labeled compounds of this invention and prodrugs thereof can generally be prepared by carrying out the procedures disclosed herein by substituting a readily available isotopically labeled reagent for a non-isotopically labeled reagent.

By way of general example and without limitation, isotopes of hydrogen, for example deuterium (²H) and tritium (³H) may optionally be used anywhere in described structures that achieves the desired result. Alternatively or in addition, isotopes of carbon, e.g., ¹³C and ¹⁴C, may be used. In one embodiment, the isotopic substitution is replacing hydrogen with a deuterium at one or more locations on the molecule to improve the performance of the molecule as a drug, for example, the pharmacodynamics, pharmacokinetics, biodistribution, half-life, stability, AUC, Tmax, Cmax, etc. For example, the deuterium can be bound to carbon in allocation of bond breakage during metabolism (an alpha-deuterium kinetic isotope effect) or next to or near the site of bond breakage (a beta-deuterium kinetic isotope effect).

Isotopic substitutions, for example deuterium substitutions, can be partial or complete. Partial deuterium substitution means that at least one hydrogen is substituted with deuterium. In certain embodiments, the isotope is 80, 85, 90, 95, or 99% or more enriched in an isotope at any location of interest. In some embodiments, deuterium is 80, 85, 90, 95, or 99% enriched at a desired location. Unless otherwise stated, the enrichment at any point is above natural abundance, and in an embodiment is enough to alter a detectable property of the compounds as a drug in a human.

The compounds of the present disclosure may form a solvate with solvents (including water). Therefore, in one embodiment, the invention includes a solvated form of the active compound. The term “solvate” refers to a molecular complex of a compound of the present invention (including a salt thereof) with one or more solvent molecules. Non-limiting examples of solvents are water, ethanol, dimethyl sulfoxide, acetone and other common organic solvents. The term “hydrate” refers to a molecular complex comprising a disclosed compound and water. Pharmaceutically acceptable solvates in accordance with the invention include those wherein the solvent of crystallization may be isotopically substituted, e.g., D₂O, d6-acetone, or d6-DMSO. A solvate can be in a liquid or solid form.

A “prodrug” as used herein means a compound which when administered to a host in vivo is converted into a parent drug. As used herein, the term “parent drug” means any of the presently described compounds herein. Prodrugs can be used to achieve any desired effect, including to enhance properties of the parent drug or to improve the pharmaceutic or pharmacokinetic properties of the parent, including to increase the half-life of the drug in vivo. Prodrug strategies provide choices in modulating the conditions for in vivo generation of the parent drug. Non-limiting examples of prodrug strategies include covalent attachment of removable groups, or removable portions of groups, for example, but not limited to, acylating, phosphorylation, phosphonylation, phosphoramidate derivatives, amidation, reduction, oxidation, esterification, alkylation, other carboxy derivatives, sulfoxy or sulfone derivatives, carbonylation, or anhydrides, among others. In certain embodiments, the prodrug renders the parent compound more lipophilic. In certain embodiments, a prodrug can be provided that has several prodrug moieties in a linear, branched, or cyclic manner. For example, non-limiting embodiments include the use of a divalent linker moiety such as a dicarboxylic acid, amino acid, diamine, hydroxycarboxylic acid, hydroxyamine, di-hydroxy compound, or other compound that has at least two functional groups that can link the parent compound with another prodrug moiety, and is typically biodegradable in vivo. In some embodiments, 2, 3, 4, or 5 prodrug biodegradable moieties are covalently bound in a sequence, branched, or cyclic fashion to the parent compound. Non-limiting examples of prodrugs according to the present disclosure are formed with: a carboxylic acid on the parent drug and a hydroxylated prodrug moiety to form an ester; a carboxylic acid on the parent drug and an amine prodrug to form an amide; an amino on the parent drug and a carboxylic acid prodrug moiety to form an amide; an amino on the parent drug and a sulfonic acid to form a sulfonamide; a sulfonic acid on the parent drug and an amino on the prodrug moiety to form a sulfonamide; a hydroxyl group on the parent drug and a carboxylic acid on the prodrug moiety to form an ester; a hydroxyl on the parent drug and a hydroxylated prodrug moiety to form an ester; a phosphonate on the parent drug and a hydroxylated prodrug moiety to form a phosphonate ester; a phosphoric acid on the parent drug and a hydroxylated prodrug moiety to form a phosphate ester; a hydroxyl on the parent drug and a phosphonate on the prodrug to form a phosphonate ester; a hydroxyl on the parent drug and a phosphoric acid prodrug moiety to form a phosphate ester; a carboxylic acid on the parent drug and a prodrug of the structure HO—(CH₂)₂—O—(C₂₋₂₄ alkyl) to form an ester; a carboxylic acid on the parent drug and a prodrug of the structure HO—(CH₂)₂—S—(C₂₋₂₄ alkyl) to form a thioester; a hydroxyl on the parent drug and a prodrug of the structure HO—(CH₂)₂—O—(C₂₋₂₄ alkyl) to form an ether; a hydroxyl on the parent drug and a prodrug of the structure HO—(CH₂)₂—O—(C₂₋₂₄ alkyl) to form an thioether; and a carboxylic acid, oxime, hydrazide, hydrazine, amine or hydroxyl on the parent compound and a prodrug moiety that is a biodegradable polymer or oligomer including but not limited to polylactic acid, polylactide-co-glycolide, polyglycolide, polyethylene glycol, polyanhydride, polyester, polyamide, or a peptide.

In some embodiments, a prodrug is provided by attaching a natural or non-natural amino acid to an appropriate functional moiety on the parent compound, for example, oxygen, nitrogen, or sulfur, and typically oxygen or nitrogen, usually in a manner such that the amino acid is cleaved in vivo to provide the parent drug. The amino acid can be used alone or covalently linked (straight, branched or cyclic) to one or more other prodrug moieties to modify the parent drug to achieve the desired performance, such as increased half-life, lipophilicity, or other drug delivery or pharmacokinetic properties. The amino acid can be any compound with an amino group and a carboxylic acid, which includes an aliphatic amino acid, alkyl amino acid, aromatic amino acid, heteroaliphatic amino acid, heteroalkyl amino acid, heterocyclic amino acid, or heteroaryl amino acid.

### Methods of Treatment

In another aspect, methods are provided for the treatment of medical disorders associated with a helicase, for example an SF3 and/or SF6 helicase, by administering a compound of Formula I, Formula II, of Formula III, or pharmaceutically acceptable salts thereof. In particular embodiments, the compounds described herein may be used in the treatment of cancer, either alone or in combination with one or more additional therapeutic agents, for example a chemotherapeutic agent. In some embodiments, the helicase comprises CMG helicase. In other embodiments, the helicase comprises HPV E1 helicase.

In another aspect, methods are provided for the treatment of medical disorders associated with a helicase, for example an SF3 and/or SF6 helicase, by administering a compound selected from:

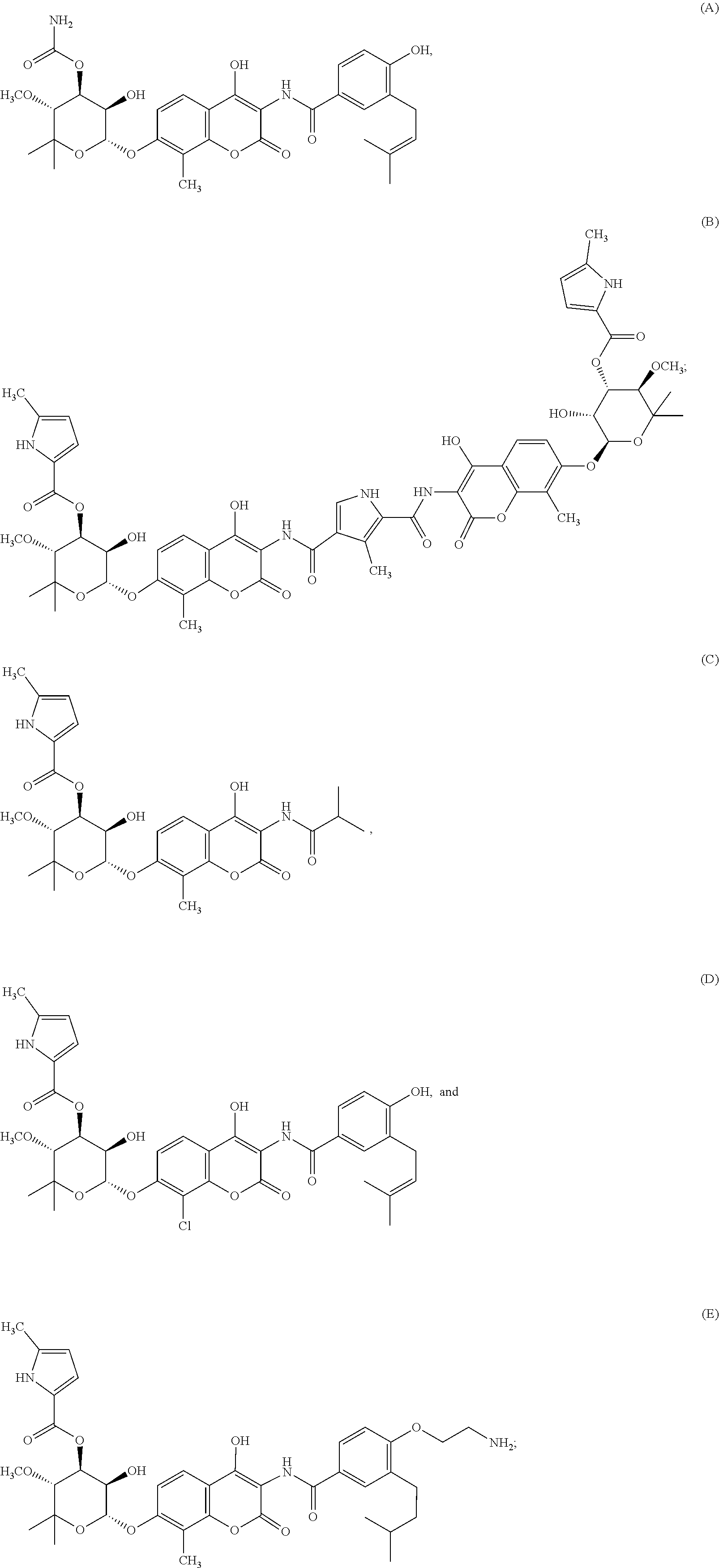

or a pharmaceutically acceptable salt thereof.

Thus in one aspect, a method is provided for treating a cancer in a subject in need thereof, the method comprising administering a therapeutically effective amount of a compound of Formula I, Formula II, or Formula III, or a compound selected from Compound A, Compound B, Compound C, Compound D, or Compound E, or a pharmaceutically acceptable salt thereof. In some embodiments, the compound of Formula I, Formula II, or Formula III, or a pharmaceutically acceptable salt thereof, or the compound selected from Compound A, Compound B, Compound C, Compound D, or Compound E, or a pharmaceutically acceptable salt thereof, is administered as a pharmaceutical composition as further described herein. In some embodiments, the subject is a human. In some embodiments, the cancer is associated with dysregulation of a helicase, for example an SF3 and/or SF6 helicase. In some embodiments, the cancer is associated with CMG helicase. In some embodiments, the cancer is associated with HPV E1 helicase.

The term “cancer” is used throughout this disclosure to refer to the pathological process that results in the formation and growth of a cancerous or malignant neoplasm, i.e., abnormal tissue (solid) or cells (non-solid) that grow by cellular proliferation, often more rapidly than normal and continues to grow after the stimuli that initiated the new growth cease. Malignant neoplasms show partial or complete lack of structural organization and functional coordination with the normal tissue and most invade surrounding tissues, can metastasize to several sites, are likely to recur after attempted removal and may cause the death of the patient unless adequately treated. As used herein, the term neoplasia is used to describe all cancerous disease states and embraces or encompasses the pathological process associated with malignant, hematogenous, ascitic and solid tumors. The cancers which may be treated by the compositions disclosed herein may comprise carcinomas, sarcomas, lymphomas, leukemias, germ cell tumors, or blastomas.

Carcinomas which may be treated by the compositions of the present disclosure include, but are not limited to, acinar carcinoma, acinous carcinoma, alveolar adenocarcinoma, carcinoma adenomatosum, adenocarcinoma, carcinoma of adrenal cortex, alveolar carcinoma, alveolar cell carcinoma, basal cell carcinoma, carcinoma basocellular, basaloid carcinoma, basosquamous cell carcinoma, breast carcinoma, bronchioalveolar carcinoma, bronchiolar carcinoma, cerebriform carcinoma, cholangiocellular carcinoma, chorionic carcinoma, colloid carcinoma, comedocarcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epibulbar carcinoma, epidermoid carcinoma, carcinoma epitheliate adenoids, carcinoma exulcere, carcinoma fibrosum, gelatinform carcinoma, gelatinous carcinoma, giant cell carcinoma, gigantocellulare, glandular carcinoma, granulose cell carcinoma, hair matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, lentivular carcinoma, carcinoma lenticulare, lipomatous carcinoma, lymphoepithelial carcinoma, carcinoma mastotoids, carcinoma medullare, medullary carcinoma, carcinoma melanodes, melanotonic carcinoma, mucinous carcinoma, carcinoma muciparum, carcinoma mucocullare, mucoepidermoid carcinoma, mucous carcinoma, carcinoma myxomatodes, masopharyngeal carcinoma, carcinoma nigrum, oat cell carcinoma, carcinoma ossificans, osteroid carcinoma, ovarian carcinoma, papillary carcinoma, periportal carcinoma, preinvasive carcinoma, prostate carcinoma, renal cell carcinoma of kidney, reserve cell carcinoma, carcinoma sarcomatodes, scheinderian carcinoma, scirrhous carcinoma, carcinoma scrota, signet-ring cell carcinoma, carcinoma simplex, small cell carcinoma, solandoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberrosum, tuberous carcinoma, verrucous carcinoma, and carcinoma vilosum.

Representative sarcomas which may be treated by the compositions of the present disclosure include, but are not limited to, liposarcomas (including myxoid liposarcomas and pleomorphic liposarcomas), leiomyosarcomas, rhabdomyosarcomas, neurofibrosarcomas, malignant peripheral nerve sheath tumors, Ewing's tumors (including Ewing's sarcoma of bone, extraskeletal or non-bone) and primitive neuroectodermal tumors (PNET), synovial sarcoma, hemangioendothelioma, fibrosarcoma, desmoids tumors, dermatofibrosarcoma protuberance (DFSP), malignant fibrous histiocytoma(MFH), hemangiopericytoma, malignant mesenchymoma, alveolar soft-part sarcoma, epithelioid sarcoma, clear cell sarcoma, desmoplastic small cell tumor, gastrointestinal stromal tumor (GIST) and osteosarcoma (also known as osteogenic sarcoma) skeletal and extra-skeletal, and chondrosarcoma.

The compositions of the present disclosure may be used in the treatment of a lymphoma. Lymphomas which may be treated include mature B cell neoplasms, mature T cell and natural killer (NK) cell neoplasms, precursor lymphoid neoplasms, Hodgkin lymphomas, and immunodeficiency-associated lymphoproliferative disorders. Representative mature B cell neoplasms include, but are not limited to, B-cell chronic lymphocytic leukemia/small cell lymphoma, B-cell prolymphocytic leukemia, lymphoplasmacytic lymphoma (such as Waldenstram macroglobulinemia), splenic marginal zone lymphoma, hairy cell leukemia, plasma cell neoplasms (such as plasma cell myeloma/multiple myeloma, plasmacytoma, monoclonal immunoglobulin deposition diseases, and heavy chain diseases), extranodal marginal zone B cell lymphoma (MALT lymphoma), nodal marginal zone B cell lymphoma, follicular lymphoma, primary cutaneous follicular center lymphoma, mantle cell lymphoma, diffuse large B cell lymphoma, diffuse large B-cell lymphoma associated with chronic inflammation, Epstein-Barr virus-positive DLBCL of the elderly, lyphomatoid granulomatosis, primary mediastinal (thymic) large B-cell lymphoma, intravascular large B-cell lymphoma, ALK+ large B-cell lymphoma, plasmablastic lymphoma, primary effusion lymphoma, large B-cell lymphoma arising in HHV8-associated multicentric Castleman's disease, and Burkitt lymphoma/leukemia. Representative mature T cell and NK cell neoplasms include, but are not limited to, T-cell prolymphocytic leukemia, T-cell large granular lymphocyte leukemia, aggressive NK cell leukemia, adult T-cell leukemia/lymphoma, extranodal NK/T-cell lymphoma, nasal type, enteropathy-associated T-cell lymphoma, hepatosplenic T-cell lymphoma, blastic NK cell lymphoma, lycosis fungoides/Sezary syndrome, primary cutaneous CD30-positive T cell lymphoproliferative disorders (such as primary cutaneous anaplastic large cell lymphoma and lymphomatoid papulosis), peripheral T-cell lymphoma not otherwise specified, angioimmunoblastic T cell lymphoma, and anaplastic large cell lymphoma. Representative precursor lymphoid neoplasms include B-lymphoblastic leukemia/lymphoma not otherwise specified, B-lymphoblastic leukemia/lymphoma with recurrent genetic abnormalities, or T-lymphoblastic leukemia/lymphoma. Representative Hodgkin lymphomas include classical Hodgkin lymphomas, mixed cellularity Hodgkin lymphoma, lymphocyte-rich Hodgkin lymphoma, and nodular lymphocyte-predominant Hodgkin lymphoma.

The compositions of the present disclosure may be used in the treatment of a Leukemia. Representative examples of leukemias include, but are not limited to, acute lymphoblastic leukemia (ALL), chronic lymphocytic leukemia (CLL), acute myelogenous leukemia (AML), chronic myelogenous leukemia (CML), hairy cell leukemia (HCL), T-cell prolymphocytic leukemia, adult T-cell leukemia, clonal eosinophilias, and transient myeloproliferative disease.

The compositions of the present disclosure may be used in the treatment of a germ cell tumor, for example germinomatous (such as germinoma, dysgerminoma, and seminoma), non germinomatous (such as embryonal carcinoma, endodermal sinus tumor, choriocarcinoma, teratoma, polyembryoma, and gonadoblastoma) and mixed tumors.

The compositions of the present disclosure may be used in the treatment of blastomas, for example hepatoblastoma, medulloblastoma, nephroblastoma, neuroblastoma, pancreatoblastoma, pleuropulmonary blastoma, retinoblastoma, and glioblastoma multiforme.

Representative cancers which may be treated include, but are not limited to: bone and muscle sarcomas such as chondrosarcoma, Ewing's sarcoma, malignant fibrous histiocytoma of bone/osteosarcoma, osteosarcoma, rhabdomyosarcoma, and heart cancer; brain and nervous system cancers such as astrocytoma, brainstem glioma, pilocytic astrocytoma, ependymoma, primitive neuroectodermal tumor, cerebellar astrocytoma, cerebral astrocytoma, glioma, medulloblastoma, neuroblastoma, oligodendroglioma, pineal astrocytoma, pituitary adenoma, and visual pathway and hypothalamic glioma; breast cancers including invasive lobular carcinoma, tubular carcinoma, invasive cribriform carcinoma, medullary carcinoma, male breast cancer, Phyllodes tumor, and inflammatory breast cancer; endocrine system cancers such as adrenocortical carcinoma, islet cell carcinoma, multiple endocrine neoplasia syndrome, parathyroid cancer, phemochromocytoma, thyroid cancer, and Merkel cell carcinoma; eye cancers including uveal melanoma and retinoblastoma; gastrointestinal cancers such as anal cancer, appendix cancer, cholangiocarcinoma, gastrointestinal carcinoid tumors, colon cancer, extrahepatic bile duct cancer, gallbladder cancer, gastric cancer, gastrointestinal stromal tumor, hepatocellular cancer, pancreatic cancer, and rectal cancer; genitourinary and gynecologic cancers such as bladder cancer, cervical cancer, endometrial cancer, extragonadal germ cell tumor, ovarian cancer, ovarian epithelial cancer, ovarian germ cell tumor, penile cancer, renal cell carcinoma, renal pelvis and ureter transitional cell cancer, prostate cancer, testicular cancer, gestational trophoblastic tumor, urethral cancer, uterine sarcoma, vaginal cancer, vulvar cancer, and Wilms tumor; head and neck cancers such as esophageal cancer, head and neck cancer, nasopharyngeal carcinoma, oral cancer, oropharyngeal cancer, paranasal sinus and nasal cavity cancer, pharyngeal cancer, salivary gland cancer, and hypopharyngeal cancer; hematopoietic cancers such as acute biphenotypic leukemia, acute eosinophilic leukemia, acute lymphoblastic leukemia, acute myeloid leukemia, acute myeloid dendritic cell leukemia, AIDS-related lymphoma, anaplastic large cell lymphoma, angioimmunoblastic T-cell lymphoma, B-cell prolymphocytic leukemia, Burkitt's lymphoma, chronic lymphocytic leukemia, chronic myelogenous leukemia, cutaneous T-cell lymphoma, diffuse large B-cell lymphoma, follicular lymphoma, hairy cell leukemia, hepatosplenic T-cell lymphoma, Hodgkin's lymphoma, hairy cell leukemia, intravascular large B-cell lymphoma, large granular lymphocytic leukemia, lymphoplasmacytic lymphoma, lymphomatoid granulomatosis, mantle cell lymphoma, marginal zone B-cell lymphoma, Mast cell leukemia, mediastinal large B cell lymphoma, multiple myeloma/plasma cell neoplasm, myelodysplastic syndroms, mucosa-associated lymphoid tissue lymphoma, mycosis fungoides, nodal marginal zone B cell lymphoma, non-Hodgkin lymphoma, precursor B lymphoblastic leukemia, primary central nervous system lymphoma, primary cutaneous follicular lymphoma, primary cutaneous immunocytoma, primary effusion lymphoma, plasmablastic lymphoma, Sezary syndrome, splenic marginal zone lymphoma, and T-cell prolymphocytic leukemia; skin cancers such as basal cell carcinoma, squamous cell carcinoma, skin adnexal tumors (such as sebaceous carcinoma), melanoma, Merkel cell carcinoma, sarcomas of primary cutaneous origin (such as dermatofibrosarcoma protuberans), and lymphomas of primary cutaneous origin (such as mycosis fungoides); thoracic and respiratory cancers such as bronchial adenomas/carcinoids, small cell lung cancer, mesothelioma, non-small cell lung cancer, pleuropulmonary blastoma, laryngeal cancer, and thymoma or thymic carcinoma; HIV/AIDs-related cancers such as Kaposi sarcoma; epithelioid hemangioendothelioma; desmoplastic small round cell tumor; and liposarcoma.

In another aspect, a method is provided for treating cancers associated with elevated expression levels of Myc and/or elevated expression levels of Cyclin E. Elevated levels of Myc and Cyclin E have been associated overactivation of CMG helicases, leading to diminished reserve MCMs available to allow the cancer cell to successfully complete the S-phase of the cell cycle. Upon exposure of the cancer cell to a CMG helicase inhibitor such as those described herein, the cancer cell faces diminished survival and potentially cell death.

Thus, in one aspect, a method is provided for treating a cancer in a subject in need thereof, the method comprising:
(a) determining whether the cancer is characterized by elevated Myc expression and/or elevated Cyclin E expression; and
(b) if the cancer is determined to be characterized by elevated Myc expression and/or elevated Cyclin E expression in (a), administering a therapeutically effective amount of a compound of Formula I, Formula II, or Formula III, or a compound selected from Compound A, Compound B, Compound C, Compound D, or Compound E, or a pharmaceutically acceptable salt thereof, either alone or in combination with one or more additional therapeutic agents (such as a chemotherapeutic or cytotoxic agent).

In another aspect, a method of treating a cancer associated with elevated Myc expression and/or elevated Cyclin E expression is provided comprising administering a therapeutically effective amount of a compound of Formula I, Formula II, or Formula III, or a compound selected from Compound A, Compound B, Compound C, Compound D, or Compound E or a pharmaceutically acceptable salt thereof, either alone or in combination with one or more additional therapeutic agents (such as a chemotherapeutic or cytotoxic agent).

In yet another aspect, a method is provided for inhibiting CMG helicase in a eukaryotic cell comprising contacting the cell with an effective amount of a compound of Formula I, Formula II, or Formula III, or a compound selected from Compound A, Compound B, Compound C, Compound D, or Compound E, or a pharmaceutically acceptable salt thereof, as described herein. In some embodiments, the eukaryotic cell is a human cell.

In yet another aspect, a method for treating cancer in a subject in need thereof is provided, the method comprising:
(a) determining whether the cancer is associated with one or more signs of replicative stress; and
(b) if the cancer is determined to be associated with one or more signs of replicative stress, administering a therapeutically effective amount of a compound of Formula I, Formula II, or Formula III, or a compound selected from Compound A, Compound B, Compound C, Compound D, or Compound E, or a pharmaceutically acceptable salt thereof.

In another aspect, a method is provided for treating cancer in a subject in need thereof, wherein the cancer has been previously determined to be associated with one or more signs of replicative stress, the method comprising administering a therapeutically effective amount of a compound of Formula I, Formula II, or Formula III, or a compound selected from Compound A, Compound B, Compound C, Compound D, or Compound E, or a pharmaceutically acceptable salt thereof.

In some embodiments, the one or more signs of replicative stress may comprise Myc overexpression, CyclinE overexpression, Rb loss, p53 loss, PolQ overexpression, or combinations thereof. In some embodiments, the one or more signs or replicative stress comprises Myc overexpression. In some embodiments, the one or more signs or replicative stress comprises CyclinE overexpression. In some embodiments, the one or more signs or replicative stress comprises Rb loss. In some embodiments, the one or more signs or replicative stress comprises p53 loss. In some embodiments, the one or more signs or replicative stress comprises PolQ overexpression.

In yet another aspect, a method for treating cancer in a subject in need thereof is provided, the method comprising:
(a) determining whether the cancer harbors one or more inherited or acquired germ-line mutations; and
(b) if the cancer is determined to harbor one or more inherited or acquired germ-line mutations in (a), administering a therapeutically effective amount of a compound of Formula I, Formula II, or Formula III, or a compound selected from Compound A, Compound B, Compound C, Compound D, or Compound E, or a pharmaceutically acceptable salt thereof.

In another aspect, a method for treating cancer in a subject in need thereof is provided, wherein the cancer has been previously determined to harbor one or more inherited or acquired germ-line mutations, the method comprising administering a therapeutically effective amount of a compound of Formula I, Formula II, or Formula III, or a compound selected from Compound A, Compound B, Compound C, Compound D, or Compound E, or a pharmaceutically acceptable salt thereof.

In some embodiments, the one or more inherited or acquired germ-line mutations may comprise loss of: p53, Rb, BRCA1, BRCA2, ATM, a xeroderma pigmentosum gene (such as XPA, XPB, XPC, XPD, XPE, XPF, or XPG), a mismatch repair gene (such as MSH2, MLH1, MSH6, PMS2), WRN, BLM, a Fanconi anemia gene (such as FANCA, FANCB, FANCC, FANCD2, FANCE, FANCF, FANCG, FANCI, FANCJ, FANCL, FANCM, FANCN, FANCO, FANCP, FANCQ, FANCT, FANCU, FANCV, or FANCW), NBS, Chek2, RecqL4, MYH, PALB2, BACH1, RAC51C, or combinations thereof.

In some embodiments, the one or more inherited or acquired germ-line mutations comprise loss of p53. In some embodiments, the one or more inherited or acquired germ-line mutations comprise loss of Rb. In some embodiments, the one or more inherited or acquired germ-line mutations comprise loss of BRCA1. In some embodiments, the one or more inherited or acquired germ-line mutations comprise loss of BRCA2. In some embodiments, the one or more inherited or acquired germ-line mutations comprise loss of ATM. In some embodiments, the one or more inherited or acquired germ-line mutations comprise loss of XPA. In some embodiments, the one or more inherited or acquired germ-line mutations comprise loss of XPB. In some embodiments, the one or more inherited or acquired germ-line mutations comprise loss of XPC. In some embodiments, the one or more inherited or acquired germ-line mutations comprise loss of XPD. In some embodiments, the one or more inherited or acquired germ-line mutations comprise loss of XPE. In some embodiments, the one or more inherited or acquired germ-line mutations comprise loss of XPF. In some embodiments, the one or more inherited or acquired germ-line mutations comprise loss of XPG. In some embodiments, the one or more inherited or acquired germ-line mutations comprise loss of MSH2. In some embodiments, the one or more inherited or acquired germ-line mutations comprise loss of MLH1. In some embodiments, the one or more inherited or acquired germ-line mutations comprise loss of MSH6. In some embodiments, the one or more inherited or acquired germ-line mutations comprise loss of PMS2. In some embodiments, the one or more inherited or acquired germ-line mutations comprise loss of WRN. In some embodiments, the one or more inherited or acquired germ-line mutations comprise loss of BLM. In some embodiments, the one or more inherited or acquired germ-line mutations comprise loss of FANCA. In some embodiments, the one or more inherited or acquired germ-line mutations comprise loss of FANCB. In some embodiments, the one or more inherited or acquired germ-line mutations comprise loss of FANCC. In some embodiments, the one or more inherited or acquired germ-line mutations comprise loss of FANCD2. In some embodiments, the one or more inherited or acquired germ-line mutations comprise loss of FANCE. In some embodiments, the one or more inherited or acquired germ-line mutations comprise loss of FANCF. In some embodiments, the one or more inherited or acquired germ-line mutations comprise loss of FANCG. In some embodiments, the one or more inherited or acquired germ-line mutations comprise loss of FANCI. In some embodiments, the one or more inherited or acquired germ-line mutations comprise loss of FANCJ. In some embodiments, the one or more inherited or acquired germ-line mutations comprise loss of FANCL. In some embodiments, the one or more inherited or acquired germ-line mutations comprise loss of FANCM. In some embodiments, the one or more inherited or acquired germ-line mutations comprise loss of FANCN. In some embodiments, the one or more inherited or acquired germ-line mutations comprise loss of FANCO. In some embodiments, the one or more inherited or acquired germ-line mutations comprise loss of FANCP. In some embodiments, the one or more inherited or acquired germ-line mutations comprise loss of FANCQ. In some embodiments, the one or more inherited or acquired germ-line mutations comprise loss of FANCT. In some embodiments, the one or more inherited or acquired germ-line mutations comprise loss of FANCU. In some embodiments, the one or more inherited or acquired germ-line mutations comprise loss of FANCV. In some embodiments, the one or more inherited or acquired germ-line mutations comprise loss of FANCW. In some embodiments, the one or more inherited or acquired germ-line mutations comprise loss of NBS. In some embodiments, the one or more inherited or acquired germ-line mutations comprise loss of Chek2. In some embodiments, the one or more inherited or acquired germ-line mutations comprise loss of RecqL4. In some embodiments, the one or more inherited or acquired germ-line mutations comprise loss of MYH. In some embodiments, the one or more inherited or acquired germ-line mutations comprise loss of PALB2. In some embodiments, the one or more inherited or acquired germ-line mutations comprise loss of BACH1. In some embodiments, the one or more inherited or acquired germ-line mutations comprise loss of RAC51C.

In an alternative aspect, a method is provided for treating an infection resulting from a papillomavirus in a subject in need thereof comprising administering a therapeutically effective amount of a compound described herein, or a pharmaceutically acceptable salt thereof, to the subject.

In some embodiments, the papillomavirus is human papillomavirus (HPV). In some embodiments, the HPV is an HPV strain selected from a strain including, but not limited to, HPV1, HPV2, HPV3, HPV4, HPV6, HPV7, HPV10, HPV11, HPV13, HPV16, HPV18, HPV22, HPV26, HPV28, HPV31, HPV32, HPV33, HPV35, HPV39, HPV42, HPV44, HPV45, HPV51, HPV52, HPV53, HPV56, HPV58, HPV59, HPV60, HPV63, HPV66, HPV68, HPV73, HPV82, or any other HPV strain which is known to result in an infection associated with a medical disorder.

In another aspect, a method is provided for treating a medical disorder associated with infection with human papillomavirus comprising administering to a subject in need thereof a therapeutically effective amount of a compound described herein, or a pharmaceutically acceptable salt thereof. In some embodiments, the medical disorder associated infection with human papillomavirus is cancer. Representative examples of medical disorders resulting from infection with HPV include, but are not limited to, common warts (associated with HPV2, HPV7, and HPV22, for example), plantar warts (associated with HPV1, HPV2, HPV4, and HPV63, for example), flat warts (associated with HPV3, HPV10, and HPV28, for example), anogenital warts (associated with HPV6, HPV11, HPV42, and HPV42, for example), genital cancers (associated with HPV16, HPV18, HPV26 HPV31, HPV33, HPV35, HPV39, HPV45, HPV51, HPV52, HPV53, HPV56, HPV58, HPV59, HPV66, HPV72, and HPV82, for example), epidermodysplasia verruciformis, focal epithelial hyperplasia (associated with HPV13 and HPV32, for example), mouth papillomas (associated with HPV6, HPV7, HPV11, HPV16, and HPV32, for example), oropharyngeal cancer (associated with HPV16, for example), verrucous cyst (associated with HPV60, for example), and laryngeal papillomatosis (associated with HPV6 and HPV11, for example).

In some embodiments of the methods described herein, the compounds of Formula I, Formula II, or Formula III, or a pharmaceutically acceptable salt thereof, or the compound selected from Compound A, Compound B, Compound C, Compound D, or Compound E, or pharmaceutically acceptable salts thereof, may be administered alone or in combination with one or more additional therapeutic agents.

In some embodiments, the one or more additional therapeutic agents may comprise an agent used to treat cancer, i.e., a cancer drug or anti-cancer agent. Exemplary cancer drugs can be selected from antimetabolite anti-cancer agents and antimitotic anti-cancer agents, and combinations thereof, to a subject. Various antimetabolite and antimitotic anti-cancer agents, including single such agents or combinations of such agents, may be employed in the methods and compositions described herein.

Antimetabolic anti-cancer agents typically structurally resemble natural metabolites, which are involved in normal metabolic processes of cancer cells such as the synthesis of nucleic acids and proteins. The antimetabolites, however, differ enough from the natural metabolites such that they interfere with the metabolic processes of cancer cells. In the cell, antimetabolites are mistaken for the metabolites they resemble, and are processed by the cell in a manner analogous to the normal compounds. The presence of the “decoy” metabolites prevents the cells from carrying out vital functions and the cells are unable to grow and survive. For example, antimetabolites may exert cytotoxic activity by substituting these fraudulent nucleotides into cellular DNA, thereby disrupting cellular division, or by inhibition of critical cellular enzymes, which prevents replication of DNA.

In one aspect, therefore, the antimetabolite anti-cancer agent is a nucleotide or a nucleotide analog. In certain aspects, for example, the antimetabolite agent may comprise purine (e.g., guanine or adenosine) or analogs thereof, or pyrimidine (cytidine or thymidine) or analogs thereof, with or without an attached sugar moiety.

Suitable antimetabolite anti-cancer agents for use in the present disclosure may be generally classified according to the metabolic process they affect, and can include, but are not limited to, analogues and derivatives of folic acid, pyrimidines, purines, and cytidine. Thus, in one aspect, the antimetabolite agent(s) is selected from the group consisting of cytidine analogs, folic acid analogs, purine analogs, pyrimidine analogs, and combinations thereof.

In one particular aspect, for example, the antimetabolite agent is a cytidine analog. According to this aspect, for example, the cytidine analog may be selected from the group consisting of cytarabine (cytosine arabinoside), azacitidine (5-azacytidine), and salts, analogs, and derivatives thereof.

In another particular aspect, for example, the antimetabolite agent is a folic acid analog. Folic acid analogs or antifolates generally function by inhibiting dihydrofolate reductase (DHFR), an enzyme involved in the formation of nucleotides; when this enzyme is blocked, nucleotides are not formed, disrupting DNA replication and cell division. According to certain aspects, for example, the folic acid analog may be selected from the group consisting of denopterin, methotrexate (amethopterin), pemetrexed, pteropterin, raltitrexed, trimetrexate, and salts, analogs, and derivatives thereof.

In another particular aspect, for example, the antimetabolite agent is a purine analog. Purine-based antimetabolite agents function by inhibiting DNA synthesis, for example, by interfering with the production of purine containing nucleotides, adenine and guanine which halts DNA synthesis and thereby cell division. Purine analogs can also be incorporated into the DNA molecule itself during DNA synthesis, which can interfere with cell division. According to certain aspects, for example, the purine analog may be selected from the group consisting of acyclovir, allopurinol, 2-aminoadenosine, arabinosyl adenine (ara-A), azacitidine, azathiprine, 8-aza-adenosine, 8-fluoro-adenosine, 8-methoxy-adenosine, 8-oxo-adenosine, cladribine, deoxycoformycin, fludarabine, gancylovir, 8-aza-guanosine, 8-fluoro-guanosine, 8-methoxy-guanosine, 8-oxo-guanosine, guanosine diphosphate, guanosine diphosphate-beta-L-2-aminofucose, guanosine diphosphate-D-arabinose, guanosine diphosphate-2-fluorofucose, guanosine diphosphate fucose, mercaptopurine (6-MP), pentostatin, thiamiprine, thioguanine (6-TG), and salts, analogs, and derivatives thereof.

In yet another particular aspect, for example, the antimetabolite agent is a pyrimidine analog. Similar to the purine analogs discussed above, pyrimidine-based antimetabolite agents block the synthesis of pyrimidine-containing nucleotides (cytosine and thymine in DNA; cytosine and uracil in RNA). By acting as “decoys,” the pyrimidine-based compounds can prevent the production of nucleotides, and/or can be incorporated into a growing DNA chain and lead to its termination. According to certain aspects, for example, the pyrimidine analog may be selected from the group consisting of ancitabine, azacitidine, 6-azauridine, bromouracil (e.g., 5-bromouracil), capecitabine, carmofur, chlorouracil (e.g. 5-chlorouracil), cytarabine (cytosine arabinoside), cytosine, dideoxyuridine, 3′-azido-3′-deoxythymidine, 3′-dideoxycytidin-2′-ene, 3′-deoxy-3′-deoxythymidin-2′-ene, dihydrouracil, doxifluridine, enocitabine, floxuridine, 5-fluorocytosine, 2-fluorodeoxycytidine, 3-fluoro-3′-deoxythymidine, fluorouracil (e.g., 5-fluorouracil (also known as 5-FU), gemcitabine, 5-methylcytosine, 5-propynylcytosine, 5-propynylthymine, 5-propynyluracil, thymine, uracil, uridine, and salts, analogs, and derivatives thereof. In one aspect, the pyrimidine analog is other than 5-fluorouracil. In another aspect, the pyrimidine analog is gemcitabine or a salt thereof.

In certain aspects, the antimetabolite agent is selected from the group consisting of 5-fluorouracil, capecitabine, 6-mercaptopurine, methotrexate, gemcitabine, cytarabine, fludarabine, pemetrexed, and salts, analogs, derivatives, and combinations thereof. In other aspects, the antimetabolite agent is selected from the group consisting of capecitabine, 6-mercaptopurine, methotrexate, gemcitabine, cytarabine, fludarabine, pemetrexed, and salts, analogs, derivatives, and combinations thereof. In one particular aspect, the antimetabolite agent is other than 5-fluorouracil. In a particularly preferred aspect, the antimetabolite agent is gemcitabine or a salt or thereof (e.g., gemcitabine HCl (Gemzar®)).

Other antimetabolite anti-cancer agents may be selected from, but are not limited to, the group consisting of acanthifolic acid, aminothiadiazole, brequinar sodium, Ciba-Geigy CGP-30694, cyclopentyl cytosine, cytarabine phosphate stearate, cytarabine conjugates, Lilly DATHF, Merrel Dow DDFC, dezaguanine, dideoxycytidine, dideoxyguanosine, didox, Yoshitomi DMDC, Wellcome EHNA, Merck & Co. EX-015, fazarabine, fludarabine phosphate, N-(2′-furanidyl)-5-fluorouracil, Daiichi Seiyaku FO-152, 5-FU-fibrinogen, isopropyl pyrrolizine, Lilly LY-188011; Lilly LY-264618, methobenzaprim, Wellcome MZPES, norspermidine, NCI NSC-127716, NCI NSC-264880, NCI NSC-39661, NCI NSC-612567, Warner-Lambert PALA, pentostatin, piritrexim, plicamycin, Asahi Chemical PL-AC, Takeda TAC-788, tiazofurin, Erbamont TIF, tyrosine kinase inhibitors, Taiho UFT and uricytin, among others.

In one aspect, the antimitotic agent is a microtubule inhibitor or a microtubule stabilizer. In general, microtubule stabilizers, such as taxanes and epothilones, bind to the interior surface of the beta-microtubule chain and enhance microtubule assembly by promoting the nucleation and elongation phases of the polymerization reaction and by reducing the critical tubulin subunit concentration required for microtubules to assemble. Unlike microtubule inhibitors, such as the vinca alkaloids, which prevent microtubule assembly, the microtubule stabilizers, such as taxanes, decrease the lag time and dramatically shift the dynamic equilibrium between tubulin dimers and microtubule polymers towards polymerization. In one aspect, therefore, the microtubule stabilizer is a taxane or an epothilone. In another aspect, the microtubule inhibitor is a vinca alkaloid.

In some embodiments, the therapeutic agent may comprise a taxane or derivative or analog thereof. The taxane may be a naturally derived compound or a related form, or may be a chemically synthesized compound or a derivative thereof, with antineoplastic properties. The taxanes are a family of terpenes, including, but not limited to paclitaxel (Taxol®) and docetaxel (Taxotere®), which are derived primarily from the Pacific yew tree, Taxus brevifolia, and which have activity against certain tumors, particularly breast and ovarian tumors. In one aspect, the taxane is docetaxel or paclitaxel. Paclitaxel is a preferred taxane and is considered an antimitotic agent that promotes the assembly of microtubules from tubulin dimers and stabilizes microtubules by preventing depolymerization. This stability results in the inhibition of the normal dynamic reorganization of the microtubule network that is essential for vital interphase and mitotic cellular functions.

Also included are a variety of known taxane derivatives, including both hydrophilic derivatives, and hydrophobic derivatives. Taxane derivatives include, but are not limited to, galactose and mannose derivatives described in International Patent Application No. WO 99/18113; piperazino and other derivatives described in WO 99/14209; taxane derivatives described in WO 99/09021, WO 98/22451, and U.S. Pat. No. 5,869,680; 6-thio derivatives described in WO 98/28288; sulfenamide derivatives described in U.S. Pat. No. 5,821,263; deoxygenated paclitaxel compounds such as those described in U.S. Pat. No. 5,440,056; and taxol derivatives described in U.S. Pat. No. 5,415,869. As noted above, it further includes prodrugs of paclitaxel including, but not limited to, those described in WO 98/58927; WO 98/13059; and U.S. Pat. No. 5,824,701. The taxane may also be a taxane conjugate such as, for example, paclitaxel-PEG, paclitaxel-dextran, paclitaxel-xylose, docetaxel-PEG, docetaxel-dextran, docetaxel-xylose, and the like. Other derivatives are mentioned in “Synthesis and Anticancer Activity of Taxol Derivatives,” D. G. I. Kingston et al., Studies in Organic Chemistry, vol. 26, entitled “New Trends in Natural Products Chemistry” (1986), Atta-ur-Rabman, P. W. le Quesne, Eds. (Elsevier, Amsterdam 1986), among other references. Each of these references is hereby incorporated by reference herein in its entirety.

Various taxanes may be readily prepared utilizing techniques known to those skilled in the art (see also WO 94/07882, WO 94/07881, WO 94/07880, WO 94/07876, WO 93/23555, WO 93/10076; U.S. Pat. Nos. 5,294,637; 5,283,253; 5,279,949; 5,274,137; 5,202,448; 5,200,534; 5,229,529; and EP 590,267) (each of which is hereby incorporated by reference herein in its entirety), or obtained from a variety of commercial sources, including for example, Sigma-Aldrich Co., St. Louis, Mo.

Alternatively, the antimitotic agent can be a microtubule inhibitor; in one preferred aspect, the microtubule inhibitor is a vinca alkaloid. In general, the vinca alkaloids are mitotic spindle poisons. The vinca alkaloid agents act during mitosis when chromosomes are split and begin to migrate along the tubules of the mitosis spindle towards one of its poles, prior to cell separation. Under the action of these spindle poisons, the spindle becomes disorganized by the dispersion of chromosomes during mitosis, affecting cellular reproduction. According to certain aspects, for example, the vinca alkaloid is selected from the group consisting of vinblastine, vincristine, vindesine, vinorelbine, and salts, analogs, and derivatives thereof.

The antimitotic agent can also be an epothilone. In general, members of the epothilone class of compounds stabilize microtubule function according to mechanisms similar to those of the taxanes. Epothilones can also cause cell cycle arrest at the G2-M transition phase, leading to cytotoxicity and eventually apoptosis. Suitable epithiolones include epothilone A, epothilone B, epothilone C, epothilone D, epothilone E, and epothilone F, and salts, analogs, and derivatives thereof. One particular epothilone analog is an epothilone B analog, ixabepilone (Ixempra™)

In certain aspects, the antimitotic anti-cancer agent is selected from the group consisting of taxanes, epothilones, vinca alkaloids, and salts and combinations thereof. Thus, for example, in one aspect the antimitotic agent is a taxane. More preferably in this aspect the antimitotic agent is paclitaxel or docetaxel, still more preferably paclitaxel. In another aspect, the antimitotic agent is an epothilone (e.g., an epothilone B analog). In another aspect, the antimitotic agent is a vinca alkaloid.

Examples of cancer drugs that may be used in the present disclosure include, but are not limited to: thalidomide; platinum coordination complexes such as cisplatin (cis-DDP), oxaliplatin and carboplatin; anthracenediones such as mitoxantrone; substituted ureas such as hydroxyurea; methylhydrazine derivatives such as procarbazine (N-methylhydrazine, MIH); adrenocortical suppressants such as mitotane (o,p′-DDD) and aminoglutethimide; RXR agonists such as bexarotene; and tyrosine kinase inhibitors such as sunitimib and imatinib. Examples of additional cancer drugs include alkylating agents, antimetabolites, natural products, hormones and antagonists, and miscellaneous agents. Alternate names are indicated in parentheses. Examples of alkylating agents include nitrogen mustards such as mechlorethamine, cyclophosphainide, ifosfamide, melphalan sarcolysin) and chlorambucil; ethylenimines and methylmelamines such as hexamethylmelamine and thiotepa; alkyl sulfonates such as busulfan; nitrosoureas such as carmustine (BCNU), semustine (methyl-CCNU), lomustine (CCNU) and streptozocin (streptozotocin); DNA synthesis antagonists such as estramustine phosphate; and triazines such as dacarbazine (DTIC, dimethyl-triazenoimidazolecarboxamide) and temozolomide. Examples of antimetabolites include folic acid analogs such as methotrexate (amethopterin); pyrimidine analogs such as fluorouracin (5-fluorouracil, 5-FU, SFU), floxuridine (fluorodeoxyuridine, FUdR), cytarabine (cytosine arabinoside) and gemcitabine; purine analogs such as mercaptopurine (6-mercaptopurine, 6-MP), thioguanine (6-thioguanine, TG) and pentostatin (2′-deoxycoformycin, deoxycoformycin), cladribine and fludarabine; and topoisomerase inhibitors such as amsacrine. Examples of natural products include vinca alkaloids such as vinblastine (VLB) and vincristine; taxanes such as paclitaxel, protein bound paclitaxel (Abraxane) and docetaxel (Taxotere); epipodophyllotoxins such as etoposide and teniposide; camptothecins such as topotecan and irinotecan; antibiotics such as dactinomycin (actinomycin D), daunorubicin (daunomycin, rubidomycin), doxorubicin, bleomycin, mitomycin (mitomycin C), idarubicin, epirubicin; enzymes such as L-asparaginase; and biological response modifiers such as interferon alpha and interlelukin 2. Examples of hormones and antagonists include luteinising releasing hormone agonists such as buserelin; adrenocorticosteroids such as prednisone and related preparations; progestins such as hydroxyprogesterone caproate, rnedroxyprogesterone acetate and megestrol acetate; estrogens such as diethylstilbestrol and ethinyl estradiol and related preparations; estrogen antagonists such as tamoxifen and anastrozole; androgens such as testosterone propionate and fluoxymesterone and related preparations; androgen antagonists such as flutamide and bicalutamide; and gonadotropin-releasing hormone analogs such as leuprolide. Alternate names and trade-names of these and additional examples of cancer drugs, and their methods of use including dosing and administration regimens, will be known to a person versed in the art.

In some aspects, the anti-cancer agent may comprise a chemotherapeutic agent. Suitable chemotherapeutic agents include, but are not limited to, alkylating agents, antibiotic agents, antimetabolic agents, hormonal agents, plant-derived agents and their synthetic derivatives, anti-angiogenic agents, differentiation inducing agents, cell growth arrest inducing agents, apoptosis inducing agents, cytotoxic agents, agents affecting cell bioenergetics i.e., affecting cellular ATP levels and molecules/activities regulating these levels, biologic agents, e.g., monoclonal antibodies, kinase inhibitors and inhibitors of growth factors and their receptors, gene therapy agents, cell therapy, e.g., stem cells, or any combination thereof. According to some aspects, the chemotherapeutic agent is selected from the group consisting of cyclophosphamide, chlorambucil, melphalan, mechlorethamine, ifosfamide, busulfan, lomustine, streptozocin, temozolomide, dacarbazine, cisplatin, carboplatin, oxaliplatin, procarbazine, uramustine, methotrexate, pemetrexed, fludarabine, cytarabine, fluorouracil, floxuridine, gemcitabine, capecitabine, vinblastine, vincristine, vinorelbine, etoposide, paclitaxel, docetaxel, doxorubicin, daunorubicin, epirubicin, idarubicin, mitoxantrone, bleomycin, mitomycin, hydroxyurea, topotecan, irinotecan, amsacrine, teniposide, erlotinib hydrochloride and combinations thereof. Each possibility represents a separate aspect of the invention.

In some embodiments, the one or more additional therapeutic agents may comprise a Chkl inhibitor, an ATR inhibitor, a Cdc7 inhibitor, or a Parp inhibitor.

In another aspect, a method for treating cancer in a subject in need thereof is provided, the method comprising:
(a) administering a therapeutically effective amount of a compound of Formula I, Formula II, or Formula III, or a compound selected from Compound A, Compound B, Compound C, Compound D, or Compound E, or a pharmaceutically acceptable salt thereof; and
(b) administering one or more additional therapeutic agents selected from a Chkl inhibitor, an ATR inhibitor, a Cdc7 inhibitor, and a Parp inhibitor.

Representative Chkl inhibitors which may be used in the above methods include, but are not limited to, AZD7762, Rabusertib (LY2603618), MK-8776 (SCH 900776), CHIR-124, PF-477736, prexasertib (LY2606368), GDC-0575, SAR-020106, CCT245737, and PD166285.

Representative ATR inhibitors which may be used in the above methods include, but are not limited to, VE-821, Berzosertib (VE-822), elimusertib (BAY-1895344), ETP-46464, CGK 733, AZ20, AZ31, ceralasertib (AZD6738), and VX-803 (M4344).

Representative examples of Cdc7 inhibitors which may be used in the above methods include, but are not limited to, XL-413, PHA-767491 (CAY10572), and LY3143921.

Representative examples of Parp inhibitors which may be used in the above methods include, but are not limited to, Olaparib, rucaparib, niraparib, talazoparib, veliparib, pamiparib (BGB-290), CEP 9722, E7016, 3-aminobenzamide, fluzoparib, AG-14361, A-966492, PJ34, UPF 1069, AZD2461, ME0328, BYK204165, BGP-15, RBN-2397, NU1025, E7449, 4-hydroxyquinazoline, NMS-P118, RBN012759, and picolinamide.

### Methods of Administration

The compounds as used in the methods described herein can be administered by any suitable method and technique presently or prospectively known to those skilled in the art. For example, the active components described herein can be formulated in a physiologically- or pharmaceutically-acceptable form and administered by any suitable route known in the art including, for example, oral and parenteral routes of administering. As used herein, the term “parenteral” includes subcutaneous, intradermal, intravenous, intramuscular, intraperitoneal, and intrasternal administration, such as by injection. Administration of the active components of their compositions can be a single administration, or at continuous and distinct intervals as can be readily determined by a person skilled in the art.

Compositions, as described herein, comprising an active compound and an excipient of some sort may be useful in a variety of medical and non-medical applications. For example, pharmaceutical compositions comprising an active compound and an excipient may be useful for the treatment or prevention of a cancer or an infection with a papillomavirus.

“Excipients” include any and all solvents, diluents or other liquid vehicles, dispersion or suspension aids, surface active agents, isotonic agents, thickening or emulsifying agents, preservatives, solid binders, lubricants and the like, as suited to the particular dosage form desired. General considerations in formulation and/or manufacture can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Exemplary excipients include, but are not limited to, any non-toxic, inert solid, semisolid or liquid filler, diluent, encapsulating material or formulation auxiliary of any type. Some examples of materials which can serve as excipients include, but are not limited to, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. As would be appreciated by one of skill in this art, the excipients may be chosen based on what the composition is useful for. For example, with a pharmaceutical composition or cosmetic composition, the choice of the excipient will depend on the route of administration, the agent being delivered, time course of delivery of the agent, etc., and can be administered to humans and/or to animals, orally, rectally, parenterally, intracisternally, intravaginally, intranasally, intraperitoneally, topically (as by powders, creams, ointments, or drops), buccally, or as an oral or nasal spray. In some embodiments, the active compounds disclosed herein are administered topically.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, etc., and/or combinations thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid. Other preservatives include tocopherol, tocopherol acetate, deteroxime mesylate, cetrimide, butylated hydroxyanisol (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary natural oils include almond, apricot kernel, avocado, babassu, bergamot, black current seed, borage, cade, chamomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Additionally, the composition may further comprise a polymer. Exemplary polymers contemplated herein include, but are not limited to, cellulosic polymers and copolymers, for example, cellulose ethers such as methylcellulose (MC), hydroxyethylcellulose (HEC), hydroxypropyl cellulose (HPC), hydroxypropyl methyl cellulose (HPMC), methylhydroxyethylcellulose (MHEC), methylhydroxypropylcellulose (MHPC), carboxymethyl cellulose (CMC) and its various salts, including, e.g., the sodium salt, hydroxyethylcarboxymethylcellulose (HECMC) and its various salts, carboxymethylhydroxyethylcellulose (CMHEC) and its various salts, other polysaccharides and polysaccharide derivatives such as starch, dextran, dextran derivatives, chitosan, and alginic acid and its various salts, carageenan, various gums, including xanthan gum, guar gum, gum arabic, gum karaya, gum ghatti, konjac and gum tragacanth, glycosaminoglycans and proteoglycans such as hyaluronic acid and its salts, proteins such as gelatin, collagen, albumin, and fibrin, other polymers, for example, polyhydroxyacids such as polylactide, polyglycolide, polyl(lactide-co-glycolide) and poly(.epsilon.-caprolactone-co-glycolide)-, carboxyvinyl polymers and their salts (e.g., carbomer), polyvinylpyrrolidone (PVP), polyacrylic acid and its salts, polyacrylamide, polyacrylic acid/acrylamide copolymer, polyalkylene oxides such as polyethylene oxide, polypropylene oxide, poly(ethylene oxide-propylene oxide), and a Pluronic polymer, polyoxy ethylene (polyethylene glycol), polyanhydrides, polyvinylalchol, polyethyleneamine and polypyrridine, polyethylene glycol (PEG) polymers, such as PEGylated lipids (e.g., PEG-stearate, 1,2-Distearoyl-sn-glycero-3-Phosphoethanolamine-N-[Methoxy(Polyethylene glycol)-1000], 1,2-Distearoyl-sn-glycero-3-Phosphoethanolamine-N-[Methoxy(Polyethylene glycol)-2000], and 1,2-Distearoyl-sn-glycero-3-Phosphoethanolamine-N-[Methoxy(Polyethylene glycol)-5000]), copolymers and salts thereof.

Additionally, the composition may further comprise an emulsifying agent. Exemplary emulsifying agents include, but are not limited to, a polyethylene glycol (PEG), a polypropylene glycol, a polyvinyl alcohol, a poly-N-vinyl pyrrolidone and copolymers thereof, poloxamer nonionic surfactants, neutral water-soluble polysaccharides (e.g., dextran, Ficoll, celluloses), non-cationic poly(meth)acrylates, non-cationic polyacrylates, such as poly (meth) acrylic acid, and esters amide and hydroxy alkyl amides thereof, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. In certain embodiments, the emulsifying agent is cholesterol.

Liquid compositions include emulsions, microemulsions, solutions, suspensions, syrups, and elixirs. In addition to the active compound, the liquid composition may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable compositions, for example, injectable aqueous or oleaginous suspensions may be formulated according to the known art using suitable dispersing or wetting agents and suspending agents. The sterile injectable preparation may also be a injectable solution, suspension, or emulsion in a nontoxic parenterally acceptable diluent or solvent, for example, as a solution in 1,3-butanediol. Among the acceptable vehicles and solvents for pharmaceutical or cosmetic compositions that may be employed are water, Ringer's solution, U.S.P. and isotonic sodium chloride solution. In addition, sterile, fixed oils are conventionally employed as a solvent or suspending medium. Any bland fixed oil can be employed including synthetic mono- or diglycerides. In addition, fatty acids such as oleic acid are used in the preparation of injectables. In certain embodiments, the particles are suspended in a carrier fluid comprising 1% (w/v) sodium carboxymethyl cellulose and 0.1% (v/v) Tween 80. The injectable composition can be sterilized, for example, by filtration through a bacteria-retaining filter, or by incorporating sterilizing agents in the form of sterile solid compositions which can be dissolved or dispersed in sterile water or other sterile injectable medium prior to use.

Compositions for rectal or vaginal administration may be in the form of suppositories which can be prepared by mixing the particles with suitable non-irritating excipients or carriers such as cocoa butter, polyethylene glycol, or a suppository wax which are solid at ambient temperature but liquid at body temperature and therefore melt in the rectum or vaginal cavity and release the particles.

Solid compositions include capsules, tablets, pills, powders, and granules. In such solid compositions, the particles are mixed with at least one excipient and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

Tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polyethylene glycols and the like.

Compositions for topical or transdermal administration include ointments, pastes, creams, lotions, gels, powders, solutions, sprays, inhalants, or patches. The active compound is admixed with an excipient and any needed preservatives or buffers as may be required.

The ointments, pastes, creams, and gels may contain, in addition to the active compound, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the active compound, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the nanoparticles in a proper medium. Absorption enhancers can also be used to increase the flux of the compound across the skin. The rate can be controlled by either providing a rate controlling membrane or by dispersing the particles in a polymer matrix or gel.

The active ingredient may be administered in such amounts, time, and route deemed necessary in order to achieve the desired result. The exact amount of the active ingredient will vary from subject to subject, depending on the species, age, and general condition of the subject, the severity of the infection, the particular active ingredient, its mode of administration, its mode of activity, and the like. The active ingredient, whether the active compound itself, or the active compound in combination with an agent, is preferably formulated in dosage unit form for ease of administration and uniformity of dosage. It will be understood, however, that the total daily usage of the active ingredient will be decided by the attending physician within the scope of sound medical judgment. The specific therapeutically effective dose level for any particular subject will depend upon a variety of factors including the disorder being treated and the severity of the disorder; the activity of the active ingredient employed; the specific composition employed; the age, body weight, general health, sex and diet of the patient; the time of administration, route of administration, and rate of excretion of the specific active ingredient employed; the duration of the treatment; drugs used in combination or coincidental with the specific active ingredient employed; and like factors well known in the medical arts.

The active ingredient may be administered by any route. In some embodiments, the active ingredient is administered via a variety of routes, including oral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, enteral, sublingual; by intratracheal instillation, bronchial instillation, and/or inhalation; and/or as an oral spray, nasal spray, and/or aerosol. In general, the most appropriate route of administration will depend upon a variety of factors including the nature of the active ingredient (e.g., its stability in the environment of the gastrointestinal tract), the condition of the subject (e.g., whether the subject is able to tolerate oral administration), etc.

The exact amount of an active ingredient required to achieve a therapeutically or prophylactically effective amount will vary from subject to subject, depending on species, age, and general condition of a subject, severity of the side effects or disorder, identity of the particular compound(s), mode of administration, and the like. The amount to be administered to, for example, a child or an adolescent can be determined by a medical practitioner or person skilled in the art and can be lower or the same as that administered to an adult.

Useful dosages of the active agents and pharmaceutical compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### Examples

We have focused on the enzymatic ATP hydrolysis steps used by the core MCM hexamers of the CMG helicase to identify ATP competitive inhibitors of AT binding and/or hydrolysis within one or more of the MCM hexamer pairs. To do this, we established a means to express and purify the human CMG helicase in our lab, and then we verified the isolated CMG is active in DNA unwinding assays. We then set up a novel assay to quantitatively measure ATP hydrolysis (ADP production) by the human CMG. This assay used reagents that are sensitive enough to read small changes in ADP production from our functioning CMG helicase in an in vitro biochemical enzyme assay. This assay relies on reading fluorescent-polarization (FP) changes from an antibody that binds to an ADP-fluorophore molecule that is displaced when the CMG produces ADP on its own. The results are obtained using a Perkin-Elmer Envision II FP plate reader. This approach is referred to as our primary screening assay. Our secondary screening assay, which is lower through-put, uses a traditional radioactive-based DNA fork unwinding method of analysis to verify whether a given potential CMGi is truly capable of inhibiting CMG elongation along DNA and subsequent fork unwinding.

Using the primary-secondary assay steps outlined above, we identified two compounds that can very effectively inhibit the human CMG helicase in vitro, and we have also obtained in vivo data arguing that the human helicase is targeted in cells by one of these compounds. The first compound identified is clorobiocin (Compound D), which was first studied almost 50 years ago as an anti-bacterial antibiotic. Clorobiocin (Compound D) is a naturally-derived antibiotic made by specific bacterial strains. At that time, it was not known what clorobiocin (Compound D) targets in bacteria, but later it was determined that gyrase and topoisomerase IV (both are type-2 topoisomerases).

We also identified a related compound called coumermycin-A1 (Compound B) as an effective CMGi. Coumermycin-A1 (Compound B) is structurally related to clorobiocin (Compound D) in that it is effectively two clorobiocin molecules attached tail-to-tail. Another related antibiotic drug, novobiocin (Compound A), was tested by our group and found not to inhibit the human CMG at any level of exposure in our biochemical assays. Therefore, there is specificity for clorobiocin (Compound D) and coumermycin-A1 (Compound B) in their ability to effectively inhibit the human CMG in biochemical assays (primary and secondary), whereas novobiocin (Compound A) fails to inhibit the CMG in any assay we used. Comparing the molecules, clorobiocin (Compound D) and coumermycin-A1 (Compound B) contain a shared chemical head group, at one end of clorobiocin (Compound D) and at both ends of coumermycin-A1 (Compound B). Novobiocin (Compound A) lacks this same head group in chemical composition. It can be interpreted from these findings that this head group likely plays a functional role in inhibiting the CMG. Furthermore, this head group of clorobiocin (Compound D) is actually known to be located in the ATP binding cleft of gyrase (based on co-crystal X-ray analyses), competing with ATP for binding to this bacterial target. This head group interacts via hydrogen bonding with a critical aspartate in gyrase, similar to how adenosine in the ATP molecule does. Our computer-based docking analysis predicts that clorobiocin (Compound D) also can interact within the ATP binding cleft in the human CMG, competing with ATP. The bacterial gyrase and human CMG appear to share some similar structural attributes in terms of how ATP enters the binding cleft, and clorobiocin (Compound D) takes advantage of this.

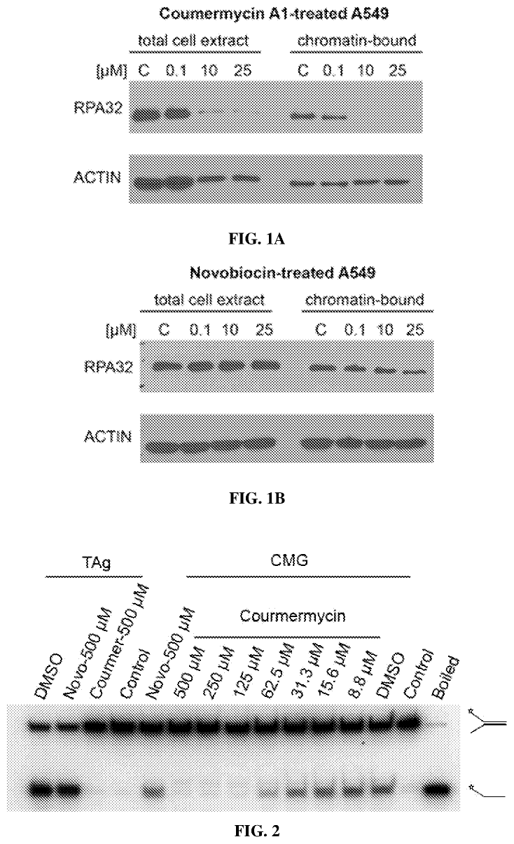

As seen in FIGS. 1A and 1B, A549 NSCLC cells were treated with 0.1, 10, or 25 μM (A) Coumermycin A1 (Compound B) or (B) Novobiocin (Compound A) for 48 hours. Cells were harvested and protein from each group isolated into total cell extracts (TCE) or chromatin-bound fractions. Standard SDS-PAGE followed by western blot was used to separate proteins and probe for RPA32 presence in TCE and bound chromatin. Actin serves as a loading control. The results show that Coumermycin A1 (Compound B) inhibits RPA loading onto DNA/chromatin, which means the helicase is inhibited (when RPA is lost from chromatin). There is also some toxicity of Coumermycin A1 (Compound B). Novobiocin (Compound A) does not inhibit RPA at all.

As shown in FIG. 2, Clorobiocin (Compound D) and Coumermycin A1 (Compound B) inhibit CMG activity at 0.5 mM compared to Novobiocin (Compound A). The human CMG helicase assay was performed with purified CMG before glycerol gradient centrifugation. Novobiocin (Compound A), Clorobiocin (Compound D), and Coumermycin A1 (Compound B) were incubated with 2 μL human CMG and 1 μL ³²P-labeled DNA forks for 30 minutes at 37 degrees Celsius. The reactions were stopped with 6× loading buffer, separated in 10% PAGE, and dried on filter paper followed by autoradiography.

As shown in FIG. 3, CMG activity was inhibited by Clorobiocin (Compound D) (81.5%) and Coumermycin A1 (Compound B) (91.7%) compared to novobiocin (Compound A) (12.5%) at 1 mM in a fluorescence-polarization assay.

Molecular targets for cancer intervention are often proteins or enzymes that are mutated or overexpressed in tumors, being different from that in normal cell counterparts. However, the CMG helicase is not mutated in any human cancers, nor is there evidence that it is over-expressed or under-expressed. In fact, clear evidence has been published indicating that CMG expression is unchanged between normal and tumor cells (1). What makes the CMG helicase an innovative and promising target for cancer intervention is that the management and availability of CMGs (assembled on DNA) is adversely affected by oncogenic signaling/proteins such as Myc, presenting a remarkable vulnerability specifically in tumors that is exploitable. This vulnerability derives from a situation where the numbers of CMG helicases known as reserve CMGs are reduced in tumor cells.

Below, Myc is used as a primary example of how reserve CMG availability is adversely affected under such an oncogenic situation. It is estimated that at least 70% of human malignancies overexpress the Myc oncogene product (Myc), sometimes 150-fold higher than normal expression SCLC, CRC, and breast carcinomas in particular display elevated Myc in 30-70% of cases, and such tumors are often difficult to treat with a poor prognosis. Clearly, the development of alternative means of clinical intervention for such tumors is of great importance. Because Myc is known to be a driving force underlying the growth and aggressiveness of these tumors, efforts to suppress Myc function have been envisioned. However, being a transcription factor, targeting Myc itself is arguably not practical. Instead, a more feasible approach is to identify innovative molecular targets that are subject to Myc's effects on these tumors, then develop therapeutic approaches against such novel targets. This example has as its focus one such innovative target: the replicative CMG helicase. As detailed below, overexpression of Myc over-activates CMG helicases, leading to an exploitable weakness in tumor cells.

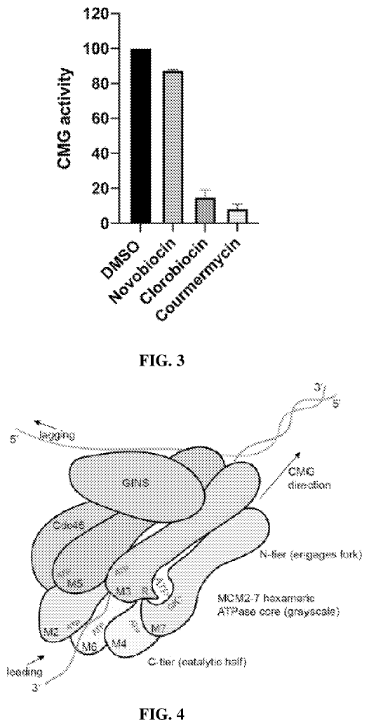

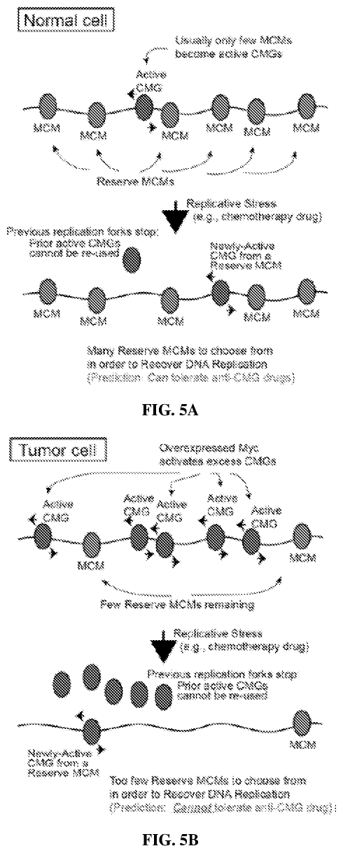

In order to appreciate the weaknesses in tumors for CMG helicases, one must understand how the CMG is assembled in two stages, and how reserve CMG helicases are generated. The CMG helicase is an 11-subunit enzyme that catalyzes the melting of DNA during DNA replication and is required for resuming replication following replicative stresses (FIG. 4). The CMG subunits include Cdc45, MCM2-7, and the GINS tetramer (subunits: Sld5, Psfl-3). In stage1 of assembly, the hexameric ATPase core of the helicase, MCM2-7, is loaded onto DNA at thousands of sites during G1-phase. At stage2 (at G1-S), Cdc45 and GINS are recruited to only a subset of MCMs to generate the few CMG helicases that will function in DNA replication. The remaining extra MCMs loaded throughout the genome act as (unused) reserves, to be converted into CMGs when problems occur and replication recovery is necessary (FIG. 5A). Problems include slowing forks due to natural topology constraints or heterochromatin, but also include chemotherapy exposure or radiation, which stop forks as well (e.g., dox, gemcitabine). Thus, MCMs are loaded onto DNA in excess and will become CMGs as needed, which is why the extra MCMs (future potential CMGs) are defined as reserves. Numerically speaking in mammalian cells, there are ˜5× excess of MCMs (reserves) loaded than are required for S-phase to be completed under unperturbed conditions.

Two additional important issues: (a) MCMs can only load during G1 and cannot load in S-phase, even if there are problems; (b) any MCM/CMG that is active cannot be re-used if replication forks stop, a new unused reserve MCM/CMG must be used to resume/recover DNA replication. This is where oncogenic situations such as Myc overexpression adversely affect the MCM/CMG process.

While Myc is well known to function as a transcription factor, it has been recently discovered that Myc also has a non-transcriptional role in (over)activating CMG helicases and stimulating excessive DNA replication (FIG. 5B). It has been uncovered in a recent study how Myc does this: a direct interaction by Myc with CMGs and GCN5/Tip60 chromatin-modifying enzymes that facilitates Cdc45/GINS binding to the MCMs to create functional CMG helicases. In this study and an older study, a domain of Myc (Myc-Box2, MB) has been identified that is required to activate the CMGs. Under conditions where Myc is overexpressed (as in SCLC), CMGs become over-activated, which depletes the number of unused reserve MCM/CMGs. Thus, whereas normal (non-tumor) cells contain sufficient unused reserve MCM/CMGs, Myc-overexpressing tumor cells do not (FIGS. 5A and 5B). The overactivation of CMGs by Myc is not itself detrimental to DNA replication/S-phase, but simply causes more active CMG helicases at any moment in time and renders inter-origin (inter-fork) distances smaller.

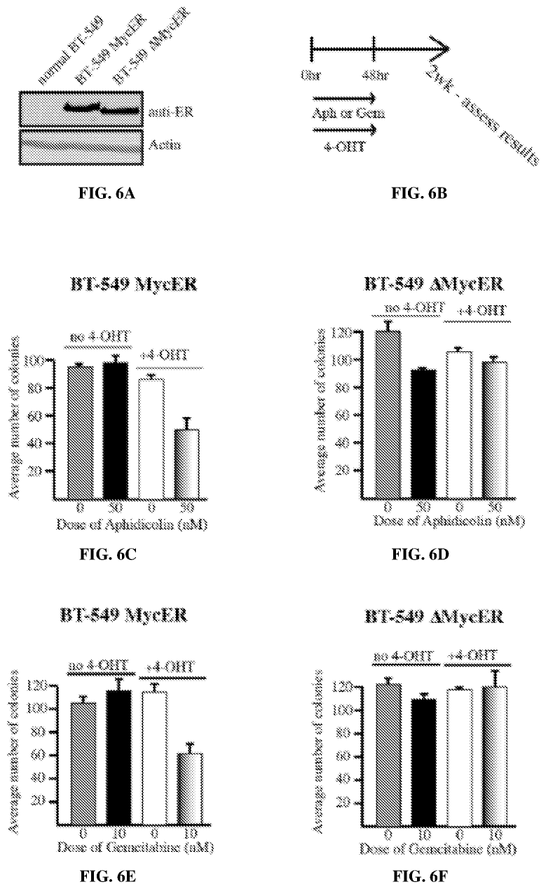

To demonstrate the chemosensitizing effects of Myc's ability to over-activate CMGs, breast tumor cells were created carrying inducible Myc protein that either can or cannot [lacking MB2] stimulate extra CMG activity and extra DNA replication. Activation of wt-Myc (but not the -MB2 mutant) is predicted to acutely create sensitivity to forkblocking drugs since wt-Myc over-activates the reserve CMGs necessary for recovery and viability. This is indeed the case (FIGS. 6A-6F). In agreement, other studies have shown that Myc-elevated tumor cells or cancers (but with long-term Myc expression) are sensitive to similar stresses. Given the ability of Myc to reduce reserve CMGs due to over-use, this provides an intriguing exploitable weakness in Myc-elevated tumor cells, and an important clinical prediction: further direct CMG inhibition (via a future drug) is predicted to (even more so) debilitate DNA replication and/or recovery from stresses in cancer cells that overexpress Myc, which is a significant portion of human malignancies (e.g., in combination with chemotherapy drugs).

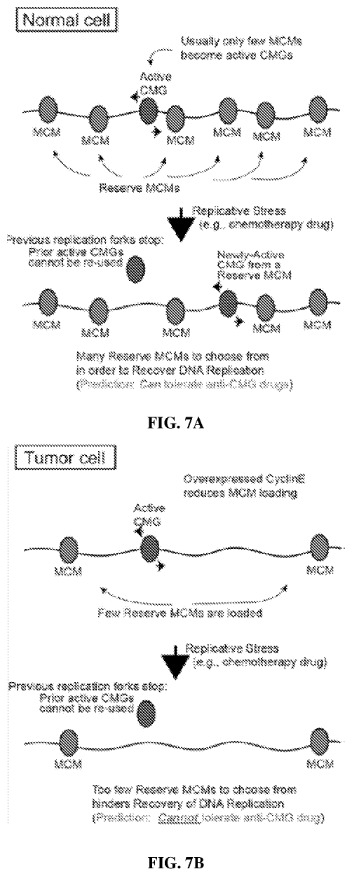

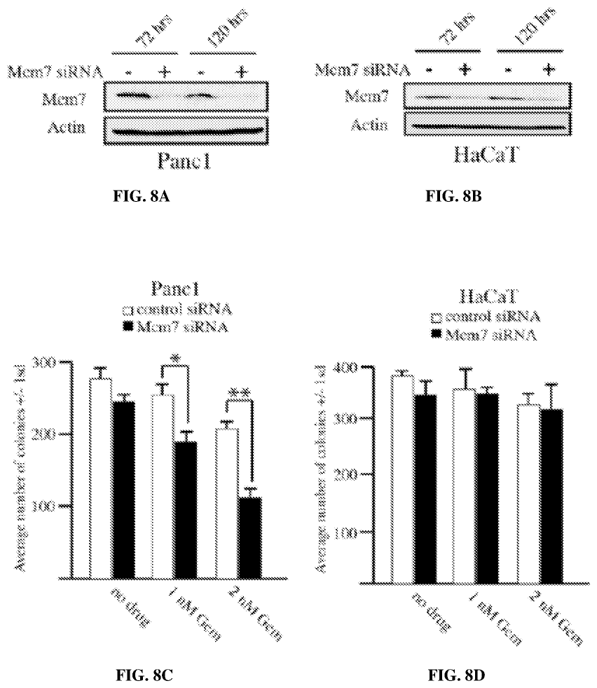

Other genetic conditions in tumor cells may also create weaknesses in CMG reserves. For example, Cyclin E overexpression (also oncogenic) is known to diminish the reserve pool of CMGs by preventing sufficient loading of precursor MCMs in G1 (24)(FIGS. 7A and 7B, compare to FIGS. 5A and 5B for Myc vs CycE differences in MCM/CMG regulation). Again, such reductions in MCM loading (or CMG overactivation by Myc) are not due to global changes in CMG/MCM subunit expression; it is due to mis-management of the assembly and activation process for CMGs by oncogenic pathways.

Whether PDAC and CRC (colorectal cancer) cells are sensitive to loss of reserve MCMs/CMGs was also investigated, but from the perspective of not knowing a genetic/oncogenic basis. A proof-of-principle study was published demonstrating that siRNA-mediated reduction of the reserve pool of MCM/CMGs (thus mimicking a future drug effect) can indeed sensitize PDAC and CRC tumor cells to replicative stresses, particularly to chemotherapy drugs (gemcitabine, 5-FU, oxaliplatin, etoposide). It was also shown that non-tumor cells are far less sensitive than PDAC cells to reserve MCM/CMG depletion in recovering from replicative stresses. As an example for one such finding, a similar loss of reserve CMGs (by siRNA) from PDAC (Panc-1) cells and non-tumor immortalized HaCaT keratinocytes results in noticeable increases in sensitivity to a fork-blocking chemotherapeutic drug (gemcitabine) for PDAC cells, but not the HaCaT cells (FIGS. 5A-5B). This indicates that PDAC are specifically compromised (for unknown genetic reasons) in their reserve CMG capacity. Most importantly, though, such results indicate that a therapeutic window exists for anti-CMG intervention (with CMG inhibitors; CMGi), where normal cells can tolerate reserve MCM/CMG inhibition (because they have reserves), while tumor cells cannot (because they are compromised for reserves). It has also been discovered that osteosarcomas (OS) and small cell lung cancer (SCLC) cells are notably sensitive to loss of MCM subunits in terms of sensitization to chemotherapy as shown above for PDAC, while non-tumor cells remain resistant to loss of MCM subunits and are not sensitized to chemotherapy when compared side-by-side with these tumor cells. Altogether, these results suggest the intriguing likelihood that many tumors may have previously unknown MCM weaknesses.

The CMG helicase represents an attractive and promising novel target for cancer intervention. The reasons for this are not that the CMG is mutated or over-/under-expressed, but rather that oncogenic signals within tumors (e.g., Myc or CycE) create an exploitable weakness in CMG reserve capacity (FIGS. 5A-5B and 7A-7B). In fact, a retrieval of data from the cBioPortal site (www.cBioPortal.org) for 42,000 human cancers reveals that CMG subunits (all 11) are not mutated, amplified, or deleted above a basal genomic rate in any such human cancers. Thus, the CMG appears to be an enzyme that simply cannot be mutated in cancers (we call the genes coding for the CMG helicase subunits ‘never-genes’ and the CMG itself a ‘neverenzyme’ for this reason). Loss-of-function mutations in the CMG would likely hinder function of all CMG complexes in a cancer cell, and the cancer cell (like normal cells) must replicate DNA and survive. Mis-management of reserve CMG pools, however, does not eradicate a tumor cell's minimal need to survive, yet provides an exploitable weakness. It can also be argued that human cancers, as a result of CMG weaknesses due to oncogenes, will not respond well to any further debilitation of the CMG given its ‘never-enzyme’ status. This is referred to conceptually as the ‘Reserve CMG Helicase Vulnerability’.

The above concepts argue strongly that the development of CMGi compounds would be beneficial in cancer intervention and management, by taking pharmacologic advantage of a specific weakness in cancers. It is predicted, for example, that CMGi drugs will provide a sensitizing effect in combination with existing chemotherapeutic drugs (fork-blocking agents such as dox, gemcitabine, topotecan) or radiation regimens, where recovery from such stresses requires full functionality of reserve CMGs, which tumors lack and CMGi drugs would further debilitate. In addition, where many tumors such as SCLC and PDAC either respond poorly to current therapies, or develop resistance to such approaches, the CMG helicase offers an intriguing alternative target with inherent weaknesses likely in such recalcitrant tumor types.

It is noted that there exists one report showing that long-term CMG suppression can promote tumorigenesis in mice. While this may seem a contra-indication toward CMGi-based interventions, one must appreciate the details of this study. In this report, all CMGs/MCMs (reserves and non-reserves) were compromised in mice from birth using a mutant Mcm4 subunit (generated by the investigators). Eventually, multiple tumor types developed due to complete and aberrant MCM/CMG functionality over a significant developmental time, from embryo to adult. The study indicated that reserve CMG/MCMs are required for normal DNA replication fidelity over long durations/cell generations, which makes sense given that sufficient reserves are necessary for cells to recover from replicative stresses (some of which may be heterochromatin or topology of DNA). In this study, the mice simply had no fully-functional reserves at any time (in any cells) in their lifetimes. However, the study cautions that any clinical cancer regimens involving CMGi (future) drugs would need to rely on approaches using such drugs for short durations, or in multiple intervals, with other drugs so as not to cause long-term damage to normal cells. Even traditional chemotherapy drugs such as crosslinking or alkylating agents cannot be used for long clinical durations.

Perhaps just as important as clinical utility, the ability to identify chemical probes targeting the CMG offers a separate opportunity to enhance basic mechanistic cancer research aimed at interrogating how the CMG is regulated biochemically, and in cancer biology. There currently exist no chemical probes against the CMG. Chemical probes will undoubtedly also be useful in structure-activity studies aimed at understanding CMG catalysis, which will provide a framework for informing drug development.

For such a promising and innovative molecular target, the CMG currently has no pharmacologic agents or chemical probes in existence that can inhibit its function. The CMG helicase is a druggable, catalytic cleft-rich holo-enzyme. The hexameric MCM core contains six ATP-binding and hydrolyzing domains (FIGS. 5A-5B). Intriguingly, and perhaps of interest for future innovative drug designs, ATP hydrolysis occurs across pairs of MCM subunits, with one subunit contributing an arginine for catalysis, and the other providing the ATP binding pocket (Walker A motif; FIG. 4). Mutations in all six ATP pockets render eukaryotic organisms non-viable, but four of the six ATP domains are more deleterious to CMG helicase function than the remaining two. Cdc45 and GINS enhance ATP hydrolysis rates (300× higher V-max), but themselves have no known enzymatic functions. Current evidence suggests the CMG translocates 3′-5′ on ssDNA at forks using a ratcheting and asymmetrical mechanism to push the CMG into the melting fork. Exposed bases of the ssDNA substrate interact with amino acid residues of the MCM core of the CMG that are presented in a non-symmetrical staircase manner in the central channel of the helicase due to temporal ATP-binding, with ATP being hydrolyzed and released as ADP as ssDNA exits the channel. This example is designed to develop and further optimize orthogonal assays for a pragmatic, automated primary screening workflow that can identify and cross-verify ATPase-regulatory compounds that inhibit the function of the CMG at one or more ATP-binding clefts (i.e., CMGi discovery).

There is a study in the literature that claims to have identified an inhibitor of the CMG helicase as being the bacterial antibiotic ciprofloxacin, or derivatives. However, careful examination of this study indicates that this drug is not an actual CMG inhibitor. The study did not show direct inhibition of the yeast CMG (human CMG not studied) and concluded that cipro likely was acting via intercalation, which is only an indirect blockage of elongation that would affect a great many related processes in cells. Also, their attempt to inhibit the human CMG (hCMG) utilized HeLa cells treated with supra-physiologic doses of cipro, which stopped cell growth, but no hCMG assessment was made per se. Thus, cipro should not be considered a hCMG inhibitor. Nonetheless, a benefit of this prior study is that it teaches something important: Any screening approach used must focus on ADP measurements as a primary screening outcome of hCMG activity, to aid in identifying inhibitors that block known hCMG catalytic action (ATP hydrolysis). This is in contrast to screens that measure DNA unwinding, which tend to yield chemical hits that often act by binding nucleic acids (e.g., intercalating), and thus only indirectly inhibit the hCMG or other enzymes.

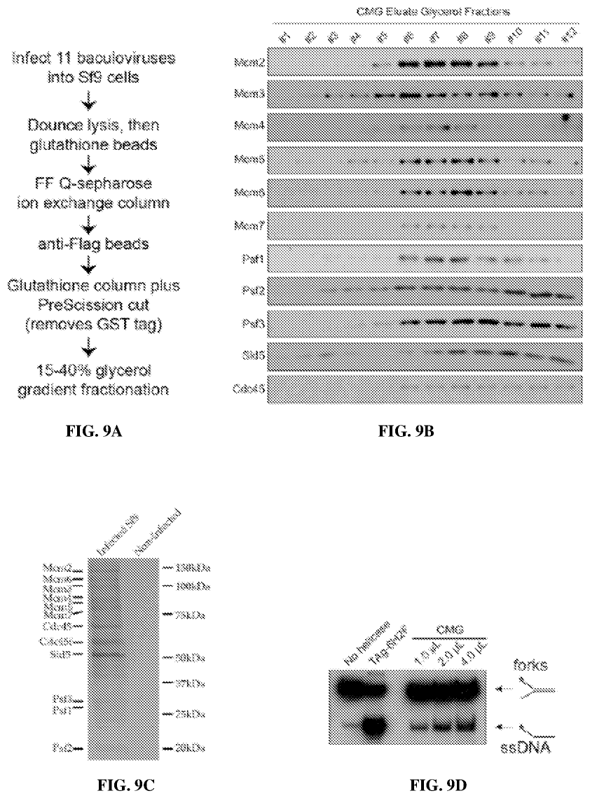

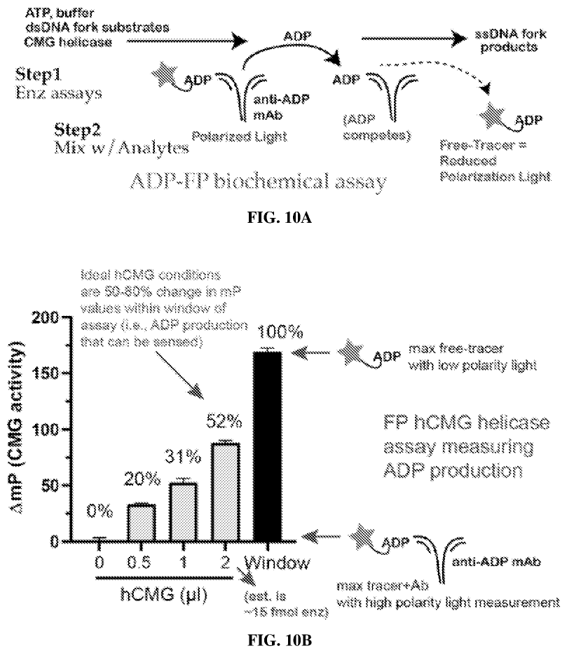

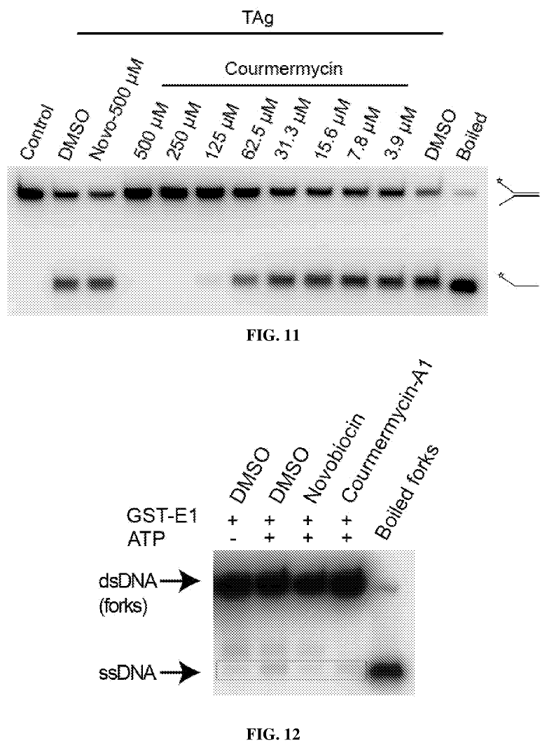

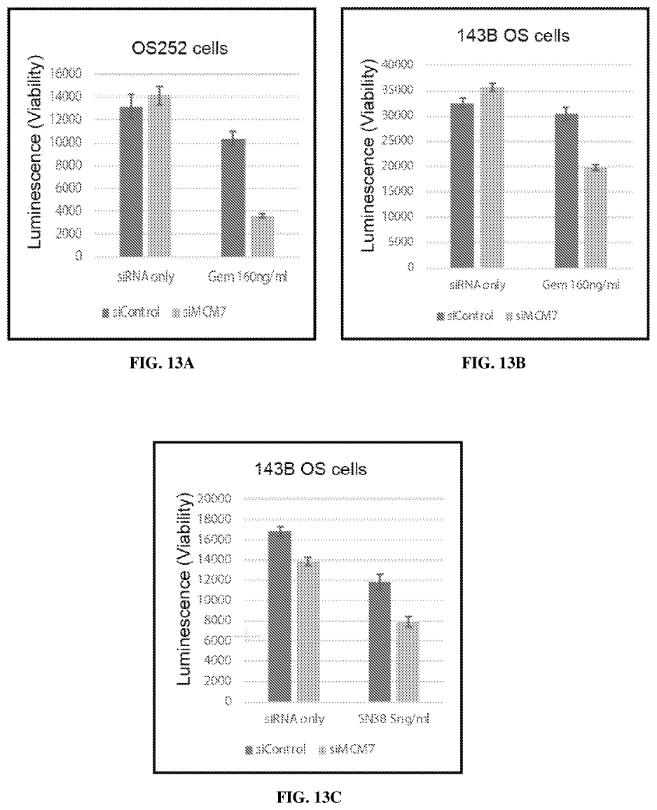

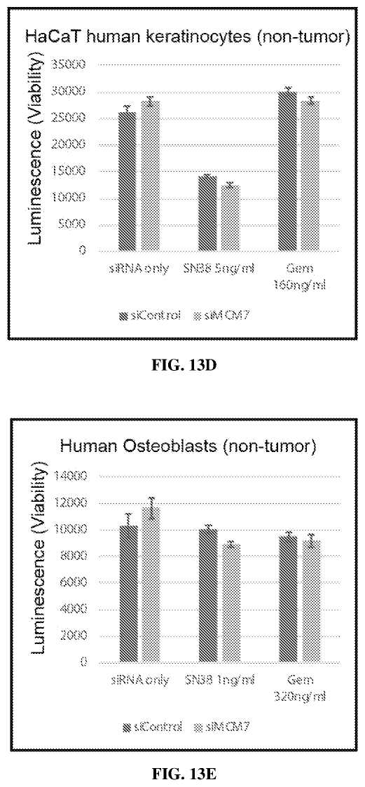

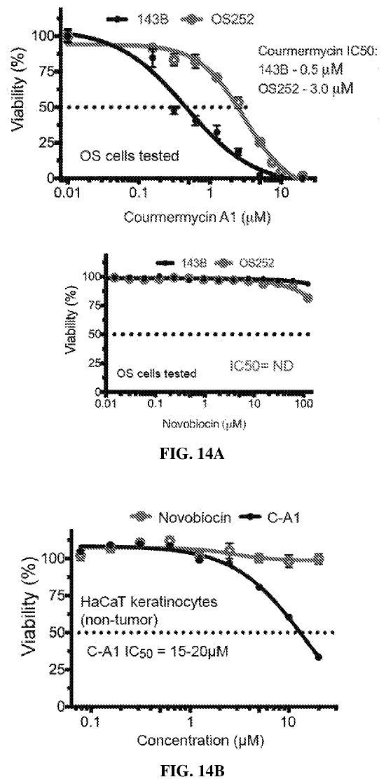

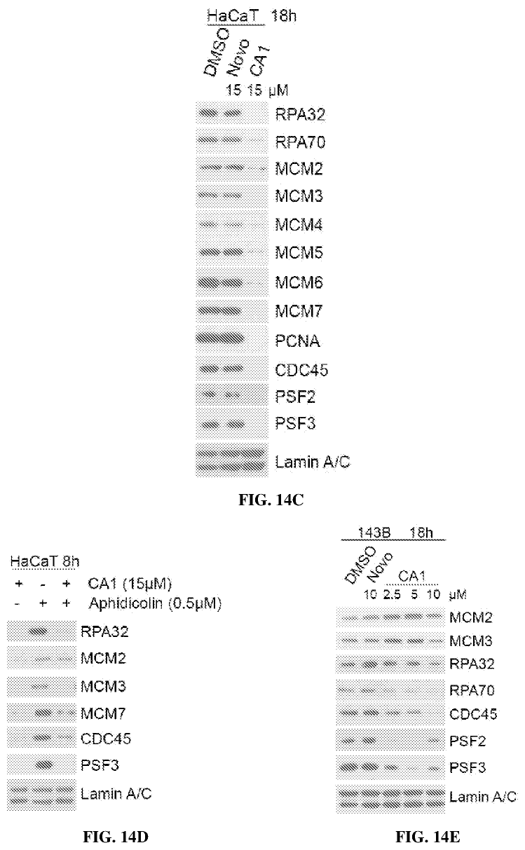

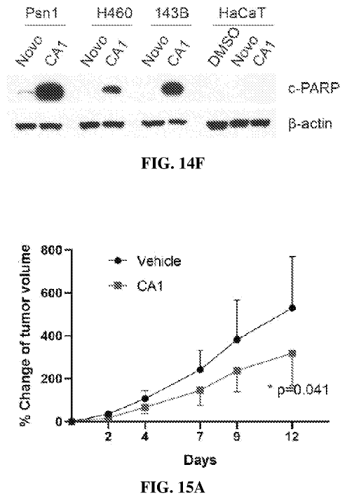

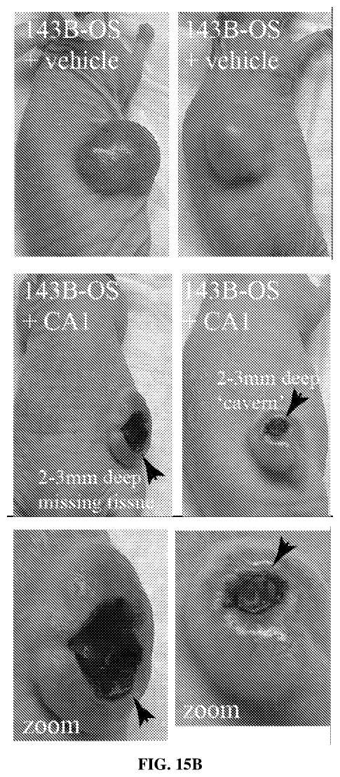

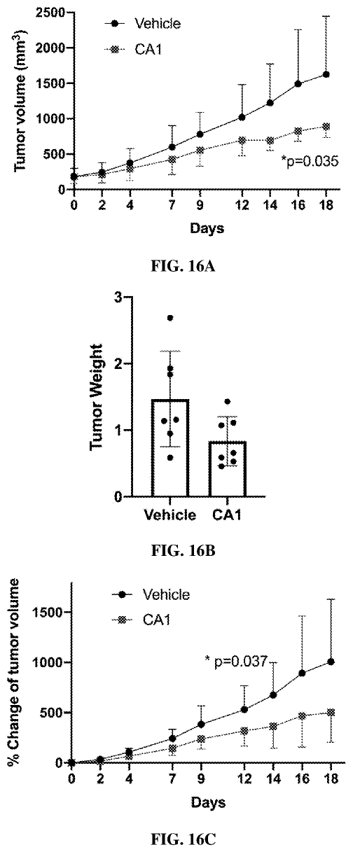

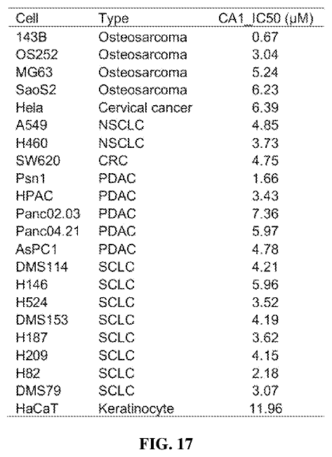

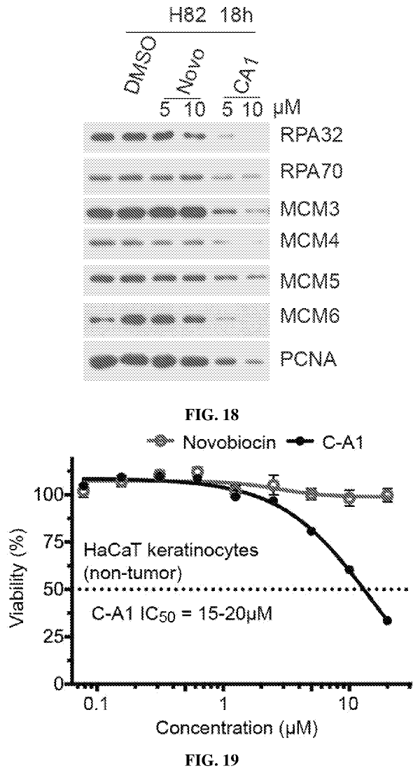

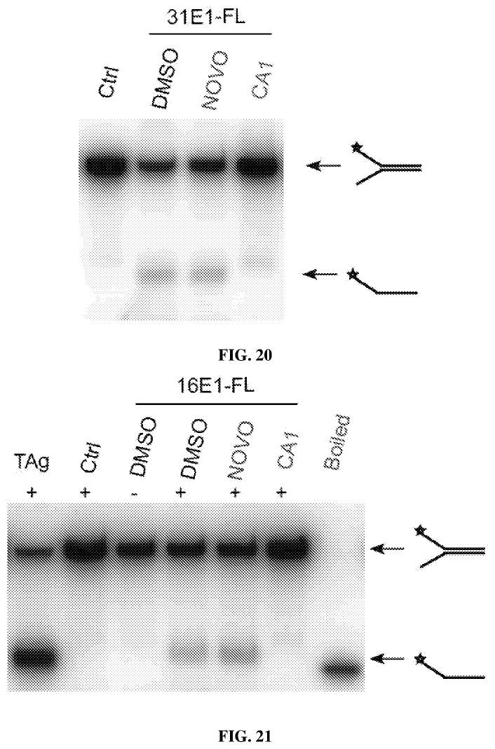

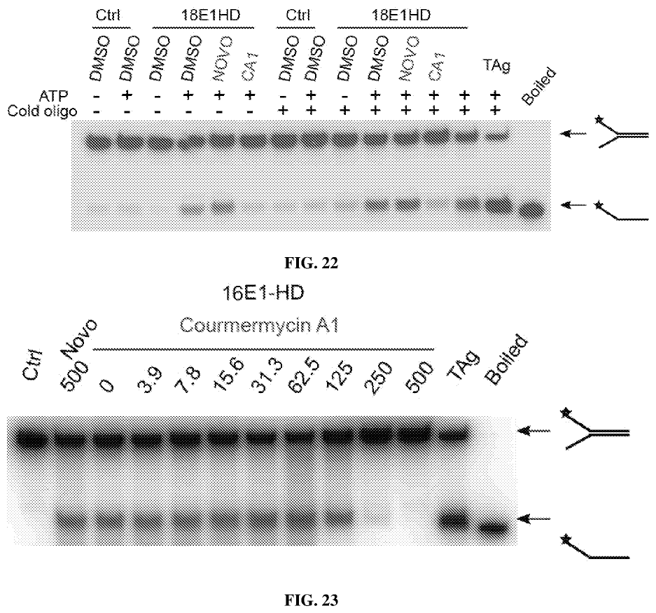

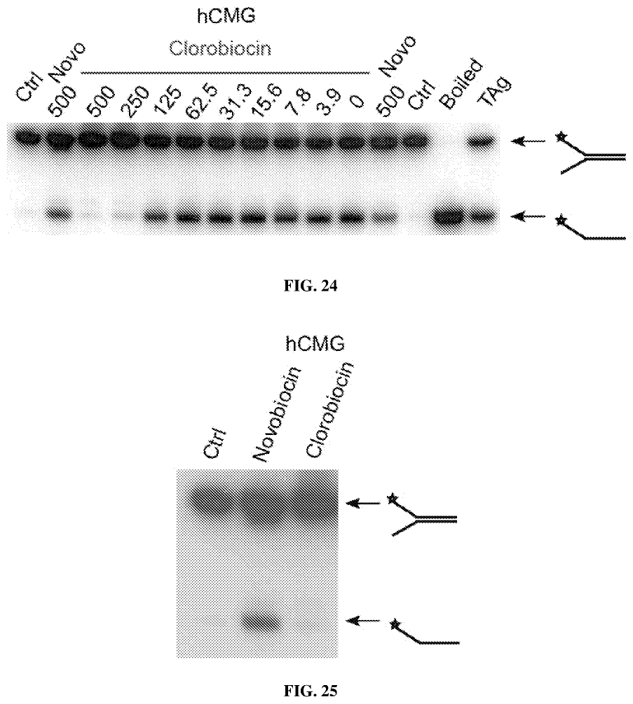

For feasibility of this example, it is shown that sufficient quantities of enzymatically active hCMG can be purified using an established approach (FIGS. 9A-9D). This method of hCMG purification was previously devised by the Hurwitz lab and involves the co-expression of all 11 subunits of the hCMG using baculoviruses and insect cells, followed by a five-step chromatography-based enrichment scheme. The steps of the purification are outlined in FIG. 9A, with Westerns on isolated fractions used to assess the completeness of hCMG subunits and enzyme recovered after the final glycerol gradient step (FIG. 9B). We also demonstrate that the stoichiometry of 11 subunits is close to 1:1 using silver-staining after purification of the hCMG (FIG. 9C). Activity is measured using a classical fork-unwinding helicase assay that relies on radioactive forks being separated, and ssDNA being recognized and quantifiable in gels using PhosphorImager analyses (FIG. 9D). SV40-derived Tag helicase was also purified as a positive control, and our active hCMG is close in forkunwinding output to that of TAg. Each prep of hCMG thus obtained is giving us -5-10 fmol of hCMG per uL of glycerol gradient fraction, with fractions totaling 150-170 uL (3-4 fractions obtained with obvious hCMG; FIG. 9B). Only 1 uL hCMG is required to easily see activity using this method (FIG. 9D). This is very consistent with that published by Hurwitz and colleagues. Given that multiple fractions produce active hCMG, this provides ample quantities of hCMG to design, calibrate, and test a workflow for hCMG chemical screening. Note that this fork-unwinding assay is actually proposed to be a secondary validation assay in screening. For secondary validation tests, an ultra-pure hCMG is desired that can be used to visualize the success or failure of a future compound in terms of hCMG inhibition of processivity (fork unwinding). As will become apparent below, it is proposed to modify the purification of the hCMG so that we can obtain even greater amounts of hCMG for primary screening using a fluorescent-based approach.

Although it is demonstrated demonstrated to purify hCMG for the purposes of feasibility of this project (Stage1 assay development), it is desired to generate higher yields of hCMG for primary screening purposes in the future, which will allow expansion of the size of chemical libraries that can be screened in Stage2/3 projects. A major step toward this goal will be to have insect cells produce far more hCMG, to increase our yields in the end. Multiple baculoviruses are used co-infected (here, 11 such CMG subunit-expressing viruses), which obviously has pitfalls: (a) not every insect cell receiving all 11 viruses, or (b) virus numbers and resultant stoichiometry are poor per insect cell (even if at least some of each virus binds), which produces partial expression of hCMG components within individual cells. These issues contribute to fewer chances for a given insect cell to receive, express, and produce hCMG holo-enzymes that we then purify. This approach can instead be modified and generate a single virus (or in worst case 2 viruses) that expresses all 11 subunits of the hCMG under the control of their own promoters. Baculoviruses are capable of simultaneously containing all of these genes, estimated to be a total insert size of 20-25 kb. The shuttle vectors we are using were obtained from Adgene and deposited by MacroLab, and are called MacroLab Bac vectors. After assembly of the multigene shuttle vector, the same Bac-to-Bac system from Invitrogen is used to produce each of the current 11 baculoviruses expressing hCMG subunits.

While the radioactive fork-unwinding assay is suitable for validation and secondary testing of potential positive hits from primary chemical library screens, it is not suitable for the primary screening itself that requires higher-throughput and can be used with fluorescent scanning equipment. Here, it is proposed to further develop a fluorescent-polarization (FP) assay for hCMG inhibitor screening found through preliminary testing tat can feasibly work with hCMG and measure ATP hydrolysis in a quantitative manner (FIGS. 7A-7B). The establishment of this FP assay along with the forkunwinding assay will present two orthogonal assays to identify and validate potential hCMG inhibitors from various library screens.

The components for the assay used for ATP hydrolysis measurements of the hCMG are produced by BellBrook Labs, and the kit is called the ADP2 Transcreener™ FP Assay. This and related screening systems have been used extensively in industry and academia for ADP product measurements of helicases (e.g., RecA helicase, RecQ-like and viral helicases) and kinases, such as Aurora kinase inhibitors by Eli Lilly, Inc. The FP assay system has thus been used to identify inhibitors for other targets that function as ATPases, and does not rely on any enzyme coupling approaches. As shown in FIGS. 10A-10B, the hCMG is incubated with ATP, buffer, and non-radioactive fork substrates for a determined length of time to allow hCMG activity to occur (or not, if an inhibitor is present). This produces ADP, which is recognized by the FP assay when free ADP competes an ADP-fluor off of a patented antibody that binds ADP with high affinity. The antibody:ADP-fluor conjugate has high polarity when measured in a Perkin Elmer Envision dual-FP plate reader (which we possess). But this polarity is reduced in a quantitative manner depending on the presence of free ADP (produced by the hCMG here), once the ADP-fluor is released from the antibody. Noteworthy, the fluor (Alexa 633) used here is in the far-red spectrum, which reduces problems during screening that can (likely) occur when chemicals themselves emit red or green wavelengths that interfere with plate reader steps (can give false positives). The system is very sensitive to even small changes in ADP production, being able to sense 0.5-2% changes quite easily (verified in our hands, using ADP-ATP standard curves). Low substrate conversion to ADP can be detected with a Z′-factor≥0.7 at 2.5% ATP conversion. The latter is achieved with a read time of less than 5 minutes. Signal stability is greater than 24 hr, and ADP-FP reagents are stable at RT for 8 hrs and tolerate 1% DMSO. Light output is also stable in the presence of up to 10% DMSO, DMF, ethanol, acetonitrile, or methanol solvents. ATP starting concentrations can be within the range of 0.1-1000 μM, and our hCMG enzyme requires 500 μM ATP for functioning near its published Km, which has been verified applies to the hCMG preparations (not shown).

Since hCMG is purified, feasibility testing of the FP assay was performed with the hCMG (FIGS. 10A-10B). Cubes, filters, and mirrors for dual-FP analysis were obtained using PE Envision II equipment, and such items were verified as functional. A window for change in millipolarizations (AmP window) is established with reagents alone, and the amount of patented antibody needed to achieve an optimal window is pre-determined in pilot titration assays using ATP at the required 500 μM level. It is then verified that the hCMG subunits themselves (non-active, without ATP added) did not alter the window (not shown). Small amounts of hCMG were tested in the FP assay (using 384-well Greiner plates), which showed titratable changes in mP values indicative of ADP being produced by our hCMG preparation (FIG. 7B). Using ˜15 fmol of hCMG enzyme (2 uL from one of our preps) a 52% ΔmP value was achieved, which is the required minimal change achieved by our hCMG as suggested by BellBrook Labs for use in future screening approaches (i.e., their suggested EC50). Using formulas BellBrook Labs supplies for their reagents, it was also calculated that the assay was working with a Z′-factor of >0.6 (calculations not shown). Optimal ΔmP can be as high as 80% change (EC80). Achieving this will also increase our Z′-factor to 0.8-0.9.

Minimally, this initial success with the FP assay and the hCMG demonstrates feasibility. However, it is proposed to improve upon this in a number of ways. First, the ability to produce more hCMG using the above viral methodology changes will certainly contribute to greater amounts of hCMG/μL added to FP reactions. Second, the hCMG is sensitive to additional proteins that can increase its activity. These include Ctf4 and Mcm1O, both of which we have incorporated into virus constructs for expression and purification. These proteins will be used in titration experiments to assess whether they can increase the activity of the hCMG in the FP assays. Third, increasing ATP concentration in reactions will increase hCMG activity, as reported. Additional testing will be performed with varying amounts of ATP to increase the potency of the hCMG in our FP assays. All of these modifications will help to further develop, calibrate, and optimize the FP assay for use with hCMG library screening.

The CMG helicase is a driver of cancer (i.e., makes replicative stress and allows cancer evolution and selection), and consequently a weakness at the same time because it is reduced in function (i.e., mismanaged) in cancers. Also, cancers are dependent upon the CMG for survival (just for growth and repair of DNA damage). This is similar, yet different, to saying oncogenes are drivers and thus targets for drugs due to cancer's dependence upon them. These are major arguments for drug discovery against the CMG (not just that it is required for DNA replication). The requirement for the CMG in DNA repair and fork stress recovery also adds another argument: the CMG is required similarly to that of ATR, which is itself a powerful rising drug target with ATR inhibition in trials already showing significant efficacy.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

1. A compound of Formula III: 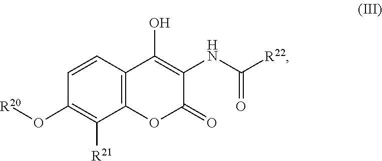
or a pharmaceutically acceptable salt thereof;
wherein:
R²⁰ is selected from R^{20a}, R^{20b}, and R^{20c};
R^{20a} is selected from 5- to 6-membered cycloalkyl, 5- to 6-membered heterocyclyl, aryl, or 5- to 6-membered heteroaryl, wherein R^{20a} is substituted with R³⁰ and is optionally substituted with one or more Y¹ groups;
R^{20b} is 5- to 6-membered heterocyclyl having N(R³¹) as a ring atom and optionally substituted with one or more Y groups;
R^{20c} is 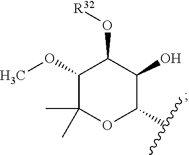
R³⁰ is selected from —NH(C═O)OR^{a1}, —O(C═O)N(R^{a2})(R^{a}), —NH(C═O)N(R^{a2})(R^{a3}), —NH(C═O)R^{a4}, and OR³²;
R³¹ is selected from —C(═O)OR^{a1}, —C(═O)N(R^{a2})(R^{a3}), —C(═S)N(R^{a2})(R^{a3}), —S(═O)N(R^{a2})(R^{a3}), —S(O)₂N(R^{a2})(R^{a3}), and —C(═O)R^{a4};
R³² is selected from —C(═O)(5- to 10-membered monocyclic or bicyclic heteroaryl) and —C(═O)(5- to 10-membered monocyclic or bicyclic heterocyclyl), each of which R³² may be optionally substituted with one or more Z¹ groups and each of which R³² may optionally contain N(Z²) as a ring heteroatom;
R²¹ is selected from C₁-C₆ alkyl and halo;
R²² is selected from C₁-C₆ alkyl, (C₃-C₆ cycloalkyl)-(C₀-C₃ alkyl)-, C₁-C₆ haloalkyl, R^{22a}, and R^{22b};
R^{22a} is 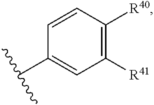
wherein R^{22a} is optionally substituted with one or more Y² groups;
R^{22b} is 5- to 6-membered heterocyclyl having N(R⁴²) as a ring atom and optionally substituted with one or more Y² groups;
R⁴⁰ is selected from hydrogen, halo, nitro, cyano, azido, —OR^{b1}, and —N(R^{b1})(R^{b2});
R⁴¹ is selected from hydrogen, C₁-C₆ alkyl, C₁-C₆ haloalkyl, C₂-C₆ alkenyl, C₂-C₆ alkynyl, (C₃-C₆ cycloalkyl)-(C₀-C₃ alkyl)-, C₁-C₆ alkoxy, and (C₃-C₆ cycloalkyl)-(C₀-C₃ alkyl)-O—;
R⁴² is selected from hydrogen, C₁-C₆ alkyl, (C₃-C₆ cycoalkyl)-(C₀-C₃ alkyl), and hydroxy(C₁-C₆ alkyl)-;
R^{a1} is selected from hydrogen, C₁-C₆ alkyl, C₂-C₆ alkenyl, C₂-C₆ alkynyl, (C₃-C₆ cycloalkyl)-(C₀-C₃ alkyl)-, (3- to 10-membered monocyclic or bicyclic heterocyclyl)-(C₀-C₃ alkyl)-, (C₆-C₁O monocyclic or bicyclic aryl)-(C₀-C₃ alkyl), and (5- to 10-membered monocyclic or bicyclic heteroaryl)-(C₀-C₃ alkyl)-, each of which may be substituted with one or more Q groups;
R^{a2} and R^{a3} are independently selected from hydrogen, C₁-C₆ alkyl, (C₃-C₆ cycloalkyl)-(C₀-C₃ alkyl)-, and (C₆-C₁O aryl)-(C₀-C₃ alkyl)-, each of which may be substituted with one or more Q groups;
R^{a4} is selected from hydrogen, C₁-C₆ alkyl, C₂-C₆ alkenyl, C₂-C₆ alkynyl, (C₃-C₆ cycloalkyl)-(C₀-C₃ alkyl)-, (3- to 10-membered monocyclic or bicyclic heterocyclyl)-(C₀-C₃ alkyl)-, (C₆-C₁O monocyclic or bicyclic aryl)-(C₀-C₃ alkyl), and (5- to 10-membered monocyclic or bicyclic heteroaryl)-(C₀-C₃ alkyl)-, each of which may be substituted with one or more Q groups;
R^{b1} and R^{b2} are independently selected at each occurrence from hydrogen, C₁-C₆ alkyl, C₂-C₆ alkenyl, C₂-C₆ alkynyl, and (C₆-C₁₀ aryl)-(C₀-C₃ alkyl)-;
Q is independently selected at each occurrence from hydrogen, halo, nitro, cyano, azido, C₁-C₆ alkyl, C₂-C₆ alkenyl, C₂-C₆ alkynyl, C₁-C₆ alkoxy, (C₃-C₆ cycloalkyl)-(C₀-C₃ alkyl)-, and (C₆-C₁₀ aryl)-(C₀-C₃ alkyl);
Y¹ is independently selected at each occurrence from hydrogen, halo, nitro, cyano, azido, —OR^{b1}, —N(R^{b1})(R^{b2}), C₁-C₆ alkyl, C₁-C₆ haloalkyl, C₂-C₆ alkenyl, C₂-C₆ alkynyl, —(C₁-C₃ alkyl)-OR^{b1}, and —(C₁-C₃ alkyl)-N(R^{b1})(R^{b2});
or two Y¹ groups on the same carbon are brought together with the carbon to which they are attached form an oxo or vinylidene group;
Y² is independently selected at each occurrence from hydrogen, halo, nitro, cyano, azido, —OR^{b1}, —N(R^{b1})(R^{b2}), C₁-C₆ alkyl, C₁-C₆ haloalkyl, C₂-C₆ alkenyl, C₂-C₆ alkynyl, —(C₁-C₃ alkyl)-OR^{b1}, and —(C₁-C₃ alkyl)-N(R^{b1})(R^{b2});
or two Y² groups on the same carbon are brought together with the carbon to which they are attached form an oxo or vinylidene group;
Z¹ is independently selected at each occurrence from halo, nitro, cyano, azido, —OR^{b1}, —N(R^{b1})(R^{b2}), C₁-C₆ alkyl, C₂-C₆ haloalkyl, C₂-C₆ alkenyl, C₂-C₆ alkynyl, (C₃-C₆ cycloalkyl)-(C₀-C₃ alkyl)-, (3- to 6-membered monocyclic or bicyclic heterocyclyl)-(C₀-C₃ alkyl)-, (C₆-C₁₀ aryl)-(C₀-C₃ alkyl)-, and (5- to 10-membered monocyclic or bicyclic heteroaryl)-(C₀-C₃ alkyl)-, each of which may be optionally substituted with one or more Q groups; and
Z² is independently selected at each occurrence from C₁-C₆ alkyl, C₂-C₆ haloalkyl, C₂-C₆ alkenyl, C₂-C₆ alkynyl, (C₃-C₆ cycloalkyl)-(C₀-C₃ alkyl)-, (3- to 6-membered monocyclic or bicyclic heterocyclyl)-(C₀-C₃ alkyl)-, (C₆-C₁₀ aryl)-(C₀-C₃ alkyl)-, and (5- to 10-membered monocyclic or bicyclic heteroaryl)-(C₀-C₃ alkyl)-, each of which may be optionally substituted with one or more Q groups.

2. (canceled)

3. The compound of claim 1, wherein R^{20a} is selected from: 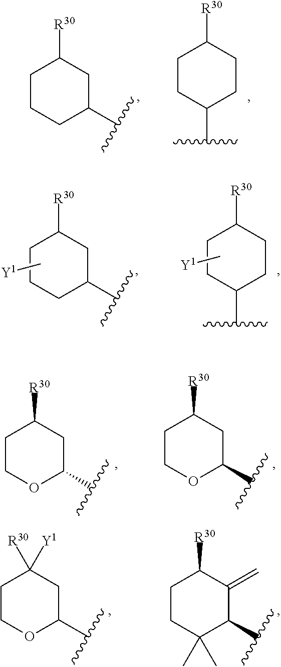 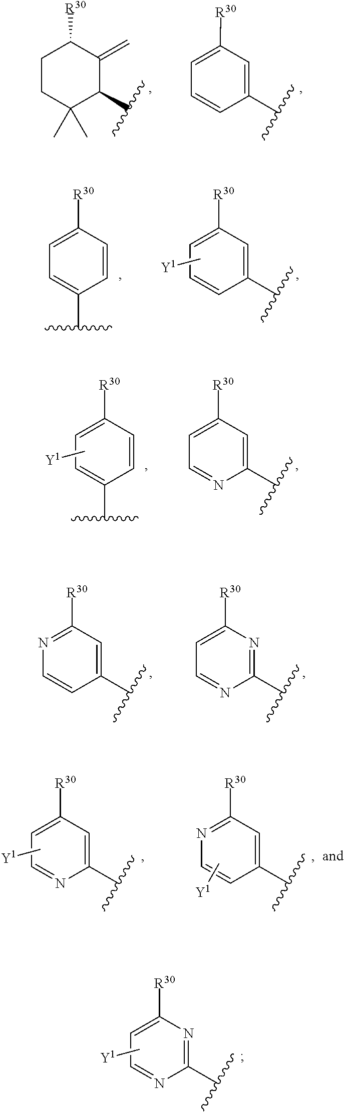 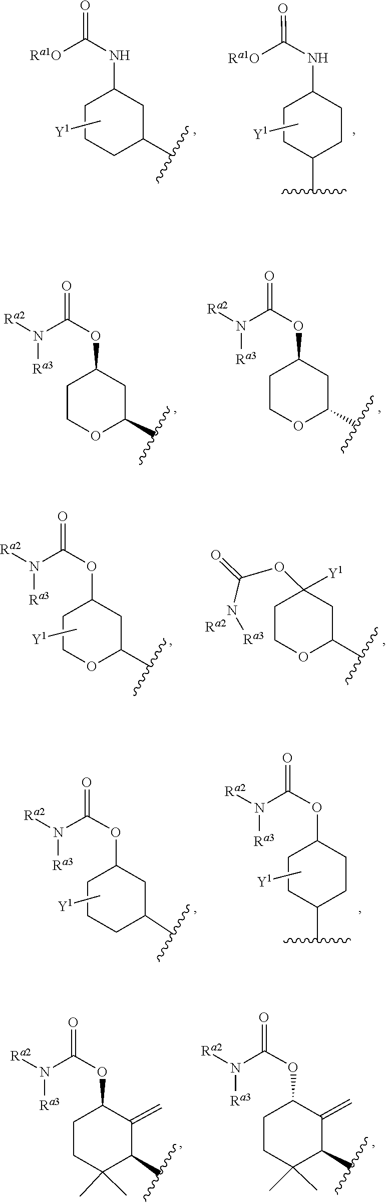 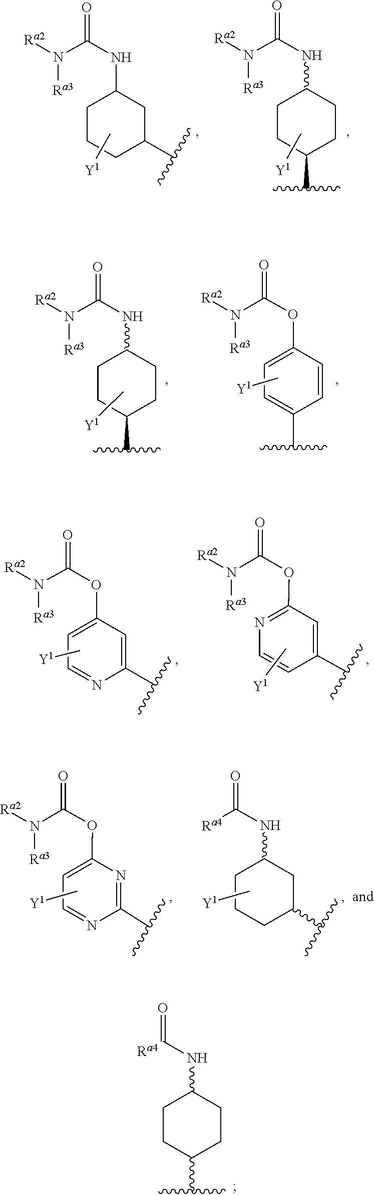
wherein R^{a1} is selected from hydrogen, methyl, ethyl, propyl, isopropyl, isobutyl, cyclopropyl, propargyl, tert-butyl, cyclopentyl, benzyl, heterocyclyl, aryl, and heteroaryl:
R^{a2} and R^{a3} are independently selected from hydrogen, methyl, ethyl, propyl, cyclopropyl, —CH₂(cyclopropyl, phenyl optionally substituted with one more substituents selected from fluoro, chloro, bromo, iodo, nitro, cyano, amino, methyl or ethyl, and benzyl; and
R^{a4} is selected from hydrogen, methyl, ethyl, propyl, isopropyl, isobutyl, cyclopropyl, propargyl, tert-butyl, cyclopentyl, benzyl, heterocyclyl, aryl, and heteroaryl.

4-7. (canceled)

8. The compound of any one of claim 3, wherein Y¹ is selected from hydrogen, hydroxy, fluoro, chloro, bromo, iodo, methoxy, amino, benzyloxy, ethoxy, propargyloxy, propargyl, methyl, ethyl, propyl, trifluoromethyl, —N(independently alkyl)₂, —NH(alkyl), or cyano.

9. (canceled)

10. The compound of claim 1, wherein R^{20b} is selected from: 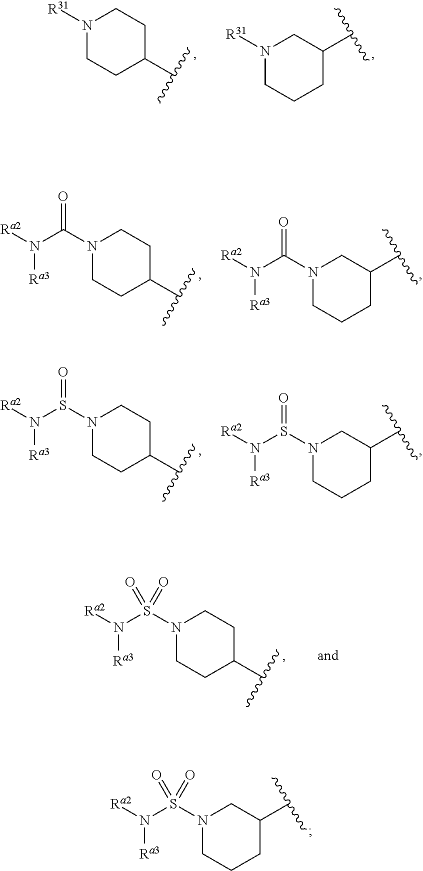
wherein R^{a2} and R^{a3} are independently selected from hydrogen, methyl, ethyl, propyl, cyclopropyl, —CH₂(cyclopropyl, phenyl optionally substituted with one more substituents selected from fluoro, chloro, bromo, iodo, nitro, cyano, amino, methyl or ethyl, and benzyl.

11-13. (canceled)

14. The compound of claim 1, wherein R²⁰ is R^{20c}, R³² is selected from: 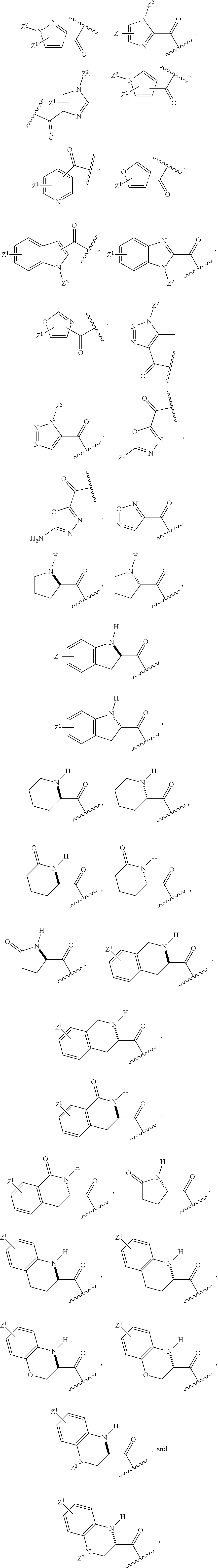
and
wherein Z¹ is selected from hydroxy, methyl, propyl, butyl, isobutyl, cyclopropyl, phenyl, propargyl, alkoxy, fluoro, chloro, bromo, or benzyl optionally substituted with one or more alkoxy or halo groups, or
wherein Z¹ is selected from hydrogen, methyl, propyl, butyl, isobutyl, cyclopropyl, phenyl optionally substituted with one or more alkyl or halo groups, propargyl, methoxy, or benzyl.

15-16. (canceled)

17. The compound of any one of claim 1, wherein R²¹ is methyl or chloro.

18. (canceled)

19. The compound of any one of claim 1, wherein R²² is selected from: 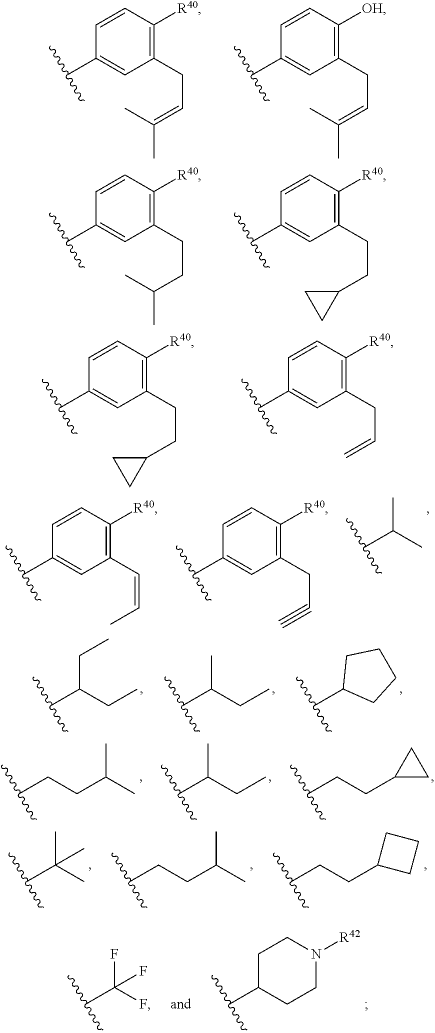
R⁴⁰ is selected from hydrogen, hydroxy, alkoxy, chloro, fluoro, cyano, —NH(alkyl), and —N(independently alkyl)₂, and
R⁴² is alkyl, cyclopropyl, or —CH₂CH₂OH.

20-27. (canceled)

28. A pharmaceutical composition comprising a compound of any one of claim 1, or a pharmaceutically acceptable salt thereof, and a pharmaceutically acceptable carrier or excipient.

29. A method for treating cancer in a subject in need thereof comprising administering to the subject a therapeutically effective amount of a compound of any one of claim 1, or a pharmaceutically acceptable salt thereof, or a pharmaceutical composition of claim 28.

30. The method of claim 29, wherein the cancer is associated with or mediated by a helicase.

31. The method of claim 30, wherein the helicase is an SF3 helicase, HPV E1 helicase, SF6 helicase, or CMG helicase.

32-34. (canceled)

35. The method of any one of claim 29, wherein the cancer is associated with overactivation of CMG helicase.

36. The method of any one of claim 29, wherein the cancer is associated with an infection by a papillomavirus.

37. The method of claim 36, wherein the papillomavirus is human papillomavirus (HPV).

38. (canceled)

39. A method for treating cancer in a subject in need thereof, wherein the cancer has been previously determined to be associated with elevated expression of Myc and/or elevated expression of Cyclin E, the method comprising administering a therapeutically effective amount of a compound of any one of claim 1, or a pharmaceutically acceptable salt thereof, or a pharmaceutical composition of claim 28.

40-42. (canceled)

43. A method of treating an infection with a papillomavirus in a subject in need thereof comprising administering a therapeutically effective amount of a compound of any one of claim 1, or a pharmaceutically acceptable salt thereof, or a pharmaceutical composition of claim 28.

44. The method of claim 43, wherein the papillomavirus comprises human papillomavirus.

45. The method of claim 44, wherein the human papillomavirus comprises a strain selected from HPV1, HPV2, HPV3, HPV4, HPV6, HPV7, HPV10, HPV11, HPV13, HPV16, HPV18, HPV22, HPV26, HPV28, HPV31, HPV32, HPV33, HPV35, HPV39, HPV42, HPV44, HPV45, HPV51, HPV52, HPV53, HPV56, HPV58, HPV59, HPV60, HPV63, HPV66, HPV68, HPV73, and HPV82.

46. (canceled)

47. The method of any one of claim 44, wherein the human papillomavirus is associated with a cancer.

48. The method of claim 47, wherein the cancer is selected from cervical cancer, vulvar cancer, vaginal cancer, penile cancer, anal cancer, rectal cancer, oropharyngeal cancer, and head and neck cancer.

49-102. (canceled)
